(19) United States
(12) Patent Application Publication
(10) Pub. No.: US 2023/0090868 A1
(43) Pub. Date: Mar. 23, 2023

(54) ANTI-CD30 ANTIBODY-DRUG CONJUGATES AND THEIR USE FOR THE TREATMENT OF NON-HODGKIN LYMPHOMA

(71) Applicant: Seagen Inc., Bothell, WA (US)

(72) Inventors: Robert Brownell SIMS, Mercer Island, WA (US); Nancy L. BARTLETT, St. Louis, MO (US)

(21) Appl. No.: 17/795,829

(22) PCT Filed: Jan. 29, 2021
(86) PCT No.: PCT/US2021/015685

§ 371 Date: Jul. 27, 2022

Related U.S. Application Data
(60) Provisional application No. 62/968,808, filed on Jan. 31, 2020.

(51) Int. Cl. A61K 47/68; A61K 31/454; A61K 39/395; A61P 35/00

(52) U.S. Cl. CPC A61K 47/6849; A61K 47/6817; A61K 47/6889; A61K 31/454; A61K 39/3955; A61P 35/00

(57) ABSTRACT

The invention provides anti-CD30 antibody-drug conjugates, such as brentuximab vedotin, and their use for the treatment of lymphoma, such as diffuse large B-cell lymphoma. The invention also provides the use of anti-CD30 antibody-drug conjugates, such as brentuximab vedotin, in combination with lenalidomide and/or anti-CD20 antibodies, such as rituximab, for the treatment of lymphoma, such as diffuse large B-cell lymphoma.

### CROSS-REFERENCE TO RELATED APPLICATION

This application claims priority to U.S. Provisional application No. 62/968,808 filed on Jan. 31, 2020, the content of which is incorporated herein by reference in its entirety.

### SUBMISSION OF SEQUENCE LISTING ON ASCII 1EXT FILE

The content of the following submission on ASCII text file is incorporated herein by reference in its entirety: a computer readable form (CRF) of the Sequence Listing (file name: 761682003340SEQLIST.TXT, date recorded: Jan. 26, 2021, size: 14 KB).

### TECHNICAL FIELD

The present application relates to anti-CD30 antibody-drug conjugates, such as brentuximab vedotin, and their use for the treatment of non-Hodgkin lymphoma, such as diffuse large B-cell lymphoma. The present application also relates to the use of anti-CD30 antibody-drug conjugates, such as brentuximab vedotin, in combination with lenalidomide and/or anti-CD20 antibodies, such as rituximab, for the treatment of non-Hodgkin lymphoma, such as diffuse large B-cell lymphoma.

### BACKGROUND

Non-Hodgkin lymphoma (NHL) is a group of blood cancers that includes all types of lymphoma except Hodgkin's lymphomas. Lymphomas are types of cancer that develop from lymphocytes, a type of white blood cell. Non-Hodgkin lymphoma includes over 60 specific types of lymphoma, including diffuse large B-cell lymphoma, follicular lymphoma, Burkitt lymphoma, mantle cell lymphoma, chronic lymphocytic leukemia, cutaneous B-cell lymphoma, and cutaneous T-cell lymphoma.

Diffuse large B-cell lymphoma (DLBCL) is the most common of the aggressive non-Hodgkin lymphomas (NHL) in the United States and accounts for approximately 30% of NHLs diagnosed annually. It is estimated that 74,200 new NHL patients will be diagnosed in the United States (US) in 2019, with 19,970 dying from the disease. DLBCL is usually aggressive, marked by rapidly growing tumors in lymph nodes, spleen, liver, bone marrow, or other organs. A very aggressive malignancy in its untreated natural history, DLBCL is a potentially curable disease, with a significant proportion of patients cured with modern chemoimmunotherapy. The current standard treatment for DLBCL is taxing on patients' quality of life, with most patients reporting much worse quality-of-life scores than the general population. Nonetheless, for those patients not cured by standard initial therapy, the prognosis remains generally poor and DLBCL still accounts for the highest number of deaths per year of all the NHL histologies.

The major clinical prognostic factors for NHL have been well described and have been incorporated into the International Prognostic Index (IPI) scoring system, which has remained applicable and relevant even in the era of rituximab-based regimens. The specific factors are: age >60 years, stage III or IV disease, performance status >2, elevated lactate dehydrogenase (LDH) levels, and extranodal involvement >1 site. These factors are combined in the IPI into 4 categories, 5-year overall survival (OS) ranging from 26% to 73% among patients treated with cyclophosphamide, doxorubicin, vincristine and prednisone (CHOP).

The current standard regimen for the management of advanced stage DLBCL is rituximab, CHOP, which achieves a cure in many subjects. Despite recent progress in improving prognosis, 20% to 50% of subjects develop relapsed/refractory (R/R) disease. Of the subjects who relapse, only 30% to 40% will respond to salvage chemotherapy. Those subjects who respond to salvage chemotherapy may receive autologous stem cell transplant (SCT) therapy, however, about 50% of those subjects will eventually relapse after autologous SCT. For subjects with R/R DLBCL, the treatment decision is determined based on various factors such as the stage, age, presence of bulky disease, and IPI. The long-term survival of these subjects with R/R DLBCL is poor with a median OS of <10 months across all standard treatments. For subjects who progress after these standard treatments, anti-CD19 chimeric antigen receptor T-cell.

Brentuximab vedotin is a CD30-directed antibody-drug conjugate (ADC) consisting of 3 components: 1) the chimeric IgG1 antibody cAC10, specific for human CD30; 2) the microtubule-disrupting agent monomethyl auristatin E (MMAE); and 3) a protease-cleavable linker that covalently attaches MMAE to cAC10. Targeted delivery of MMAE to CD30-expressing tumor cells is the primary mechanism of action of brentuximab vedotin. Binding of MMAE to tubulin disrupts the microtubule network within the cell, subsequently inducing cell cycle arrest and apoptotic death of the cell. Other nonclinical studies suggest additional contributory mechanisms of action, including antibody-dependent cellular phagocytosis; bystander effects on nearby cells in the tumor microenvironment due to released MMAE; and immunogenic cell death due to endoplasmic reticulum stress which drives exposure of immune activating molecules that can promote a T-cell response.

Lenalidomide is currently approved in the US for the treatment of relapsed or refractory mantle cell lymphoma (MCL), multiple myeloma (MM), and myelodysplastic syndromes (MDS) at a recommended starting dose of 25 mg orally once daily for 21 days every 28 days.

Lenalidomide is a more potent molecular analog of thalidomide, in vitro, lenalidomide has 3 main activities: direct anti-tumor effect, inhibition of angiogenesis, and immunomodulation. In vivo, lenalidomide induces tumor cell apoptosis directly and indirectly by inhibition of bone marrow stromal cell support, by anti-angiogenic and anti-osteoclastogenic effects, and by immunomodulatory activity. Lenalidomide has a broad range of activities that can be exploited to treat many hematologic and solid cancers. Lenalidomide has a black box warning for embryo-fetal toxicity, hematologic toxicity and venous thromboembolism.

Lenalidomide is rapidly absorbed after oral administration. After single and multiple doses of lenalidomide in subjects with MM or MDS, the maximum plasma concentrations occurred at 1 hour (approximately 0.5 to 6 hours) post-dose. Multiple doses of lenalidomide at the recommended dosage does not result in drug accumulation. Administration of a single 25 mg dose of lenalidomide with a high-fat meal in healthy subjects reduces the extent of absorption, with an approximate 20% decrease in area under the concentration-time curve and 50% decrease in maximum plasma concentration (Cₘₐₓ). In the studies where the efficacy and safety were established for lenalidomide, the drug was administered without regard to food intake. The prescribing information of lenalidomide suggested lenalidomide can be administered with or without food. The mean half-life of lenalidomide is 3 to 5 hours in subjects with MM, MDS, or MCL. Lenalidomide is not anticipated to be subjected to pharmacokinetic (PK) drug-drug interactions when co-administered with CYP inhibitors, inducers, or substrates. Despite being a weak substrate of P-gp in vitro, lenalidomide does not have clinically significant PK interactions with P-gp substrates/inhibitors in controlled studies. Renal function is the only important factor affecting lenalidomide plasma exposure, and the starting dose needs to be adjusted based on the creatinine clearance value (<60 mL/min). Age (39 to 85 years), body weight (33 to 135 kg), sex, race, and type of hematological malignancies (MM, MDS, or MCL) did not have a clinically relevant effect on lenalidomide clearance in adult subjects.

A phase 2/3 randomized, NCCN-listed study of lenalidomide monotherapy vs chemotherapy was conducted in subjects with R/R DLBCL. Lenalidomide-treated subjects had an objective response rate (ORR) of 27.5% as compared with 11.8% in investigator's choice chemotherapy (ORR were similar regardless of immunohistochemistry [IHC]-defined DLBCL subtype). Median progression-free survival (PFS) was increased in subjects receiving lenalidomide (13.6 weeks) versus investigator's choice (7.9 weeks; P=0.041), with greater improvements in non-germinal-center B-cell-like (GCB) subjects (15.1 vs 7.1 weeks, respectively; P=0.021) compared with GCB (10.1 vs 9.0 weeks, respectively; P=0.550).

Rituximab is a genetically engineered chimeric murine/human monoclonal IgG1 kappa antibody directed against the CD20 antigen. Upon binding to CD20, rituximab mediates B-cell lysis. Possible mechanisms of cell lysis include complement-dependent cytotoxicity (CDC) and antibody-dependent cell-mediated cytotoxicity (ADCC). Rituximab is marketed in the US and the European Union (EU) for use in combination chemotherapy for previously untreated NHL, including indolent B-cell lymphomas and DLBCL; it is also approved for chronic lymphocytic leukemia (CLL) and other diseases

### SUMMARY

Provided herein is a method of treating non-Hodgkin lymphoma in a subject, the method comprising administering to the subject lenalidomide, or salt or solvate thereof, and an antibody-drug conjugate that binds to CD30, wherein the antibody-drug conjugate comprises an anti-CD30 antibody or an antigen-binding fragment thereof conjugated to a monomethyl auristatin or a functional analog thereof or a functional derivative thereof. In some embodiments, the non-Hodgkin lymphoma is diffuse large B-cell lymphoma (DLBCL). In some embodiments, the DLBCL is relapsed DLBCL. In some embodiments, the DLBCL is refractory DLBCL. In some embodiments, the DLBCL is germinal-center B-cell like (GCB). In some embodiments, the DLBCL is non-GCB. In some embodiments, the subject has previously received allogenic stem cell transplant to treat the non-Hodgkin lymphoma. In some embodiments, the subject has previously received autologous stem cell transplant to treat the non-Hodgkin lymphoma. In some embodiments, the subject relapsed following stem cell transplant. In some embodiments, the subject has previously received CAR-T therapy. In some embodiments, the subject relapsed after CAR-T therapy. In some embodiments, the subject has not been previously treated with lenalidomide, or salt or solvate thereof. In some embodiments, the subject has not been previously treated with an antibody-drug conjugate that binds to CD30. In some embodiments, the non-Hodgkin lymphoma is an advanced stage non-Hodgkin lymphoma. In some embodiments, the advanced stage non-Hodgkin lymphoma is a stage 3 or stage 4 non-Hodgkin lymphoma. In some embodiments, the advanced stage non-Hodgkin lymphoma is metastatic non-Hodgkin lymphoma. In some embodiments, the non-Hodgkin lymphoma is recurrent non-Hodgkin lymphoma. In some embodiments, at least 1% of the non-Hodgkin lymphoma cells in the subject express CD30. In some embodiments, the anti-CD30 antibody of the antibody-drug conjugate comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6. In some embodiments, the anti-CD30 antibody of the antibody-drug conjugate comprises a heavy chain variable region comprising an amino acid sequence at least 85% identical to the amino acid sequence of SEQ ID NO: 7 and a light chain variable region comprising an amino acid sequence at least 85% identical to the amino acid sequence of SEQ ID NO: 8. In some embodiments, the anti-CD30 antibody of the antibody-drug conjugate comprises a heavy chain variable region comprising an amino acid sequence at least 90% identical to the amino acid sequence of SEQ ID NO: 7 and a light chain variable region comprising an amino acid sequence at least 90% identical to the amino acid sequence of SEQ ID NO: 8. In some embodiments, the anti-CD30 antibody of the antibody-drug conjugate comprises a heavy chain variable region comprising an amino acid sequence at least 95% identical to the amino acid sequence of SEQ ID NO: 7 and a light chain variable region comprising an amino acid sequence at least 95% identical to the amino acid sequence of SEQ ID NO: 8. In some embodiments, the anti-CD30 antibody of the antibody-drug conjugate comprises a heavy chain variable region comprising the amino acid sequence of SEQ ID NO: 7 and a light chain variable region comprising the amino acid sequence of SEQ ID NO: 8. In some embodiments, the anti-CD30 antibody is AC10. In some embodiments, the anti-CD30 antibody is cAC10. In some embodiments, the antibody-drug conjugate further comprises a linker between the anti-CD30 antibody or antigen-binding portion thereof and the monomethyl auristatin. In some embodiments, the linker is a cleavable peptide linker. In some embodiments, the cleavable peptide linker has a formula: -MC-vc-PAB-, wherein:

a) MC is:

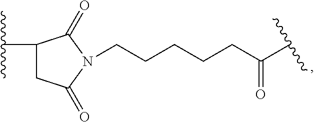 [SMILES: *C(=O)CCCCCN1C(=O)CC(*)C1=O]

b) vc is the dipeptide valine-citrulline, and

c) PAB is:

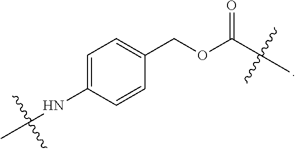 [SMILES: *Nc1ccc(COC(*)=O)cc1]

In some embodiments, the monomethyl auristatin is monomethyl auristatin E (MMAE). In some embodiments, the monomethyl auristatin is monomethyl auristatin F (MMAF). In some embodiments, the antibody-drug conjugate is brentuximab vedotin or a biosimilar thereof. In some embodiments, wherein the antibody-drug conjugate is brentuximab vedotin. In some embodiments, the lenalidomide, or salt or solvate thereof, is administered at a dose of 1 to 30 mg. In some embodiments, the lenalidomide, or salt or solvate thereof, is administered at a dose of 20 mg. In some embodiments, the lenalidomide, or salt or solvate thereof, is administered orally. In some embodiments, the lenalidomide, or salt or solvate thereof, is administered about once per day. In some embodiments, the antibody-drug conjugate is administered at a dose of 0.6 mg/kg to 2.3 mg/kg of the subject's bodyweight. In some embodiments, the antibody-drug conjugate is administered at a dose of about 0.9 mg/kg of the subject's bodyweight. In some embodiments, the antibody-drug conjugate is administered at a dose of 0.9 mg/kg of the subject's bodyweight. In some embodiments, the antibody-drug conjugate is administered at a dose of about 1.2 mg/kg of the subject's bodyweight. In some embodiments, the antibody-drug conjugate is administered at a dose of 1.2 mg/kg of the subject's bodyweight. In some embodiments, the antibody-drug conjugate is administered to subjects having a bodyweight of greater than 100 kg as if the subject had a bodyweight of 100 kg. In some embodiments, the antibody-drug conjugate is administered to the subject once about every 3 weeks. In some embodiments, the antibody-drug conjugate is administered to the subject once every 3 weeks. In some embodiments, the antibody-drug conjugate is administered to the subject on about day 1 of about a 21-day treatment cycle. In some embodiments, the antibody-drug conjugate is administered to the subject on day 1 of a 21-day treatment cycle. In some embodiments, the antibody-drug conjugate is administered by intravenous infusion. In some embodiments, the method further comprises the administration of an anti-CD20 antibody or antigen-binding fragment thereof to the subject. In some embodiments, the anti-CD20 antibody or antigen-binding fragment thereof comprises a heavy chain variable region comprising the three CDRs of SEQ ID NO:17, a light chain variable region comprising the three CDRs of SEQ ID NO:18, wherein the CDRs of the anti-CD20 antibody are defined by the Kabat numbering scheme. In some embodiments, the anti-CD20 antibody or antigen-binding fragment thereof comprises a heavy chain variable region comprising an amino acid sequence at least 85% identical to the amino acid sequence of SEQ ID NO:17 and a light chain variable region comprising an amino acid sequence at least 85% identical to the amino acid sequence of SEQ ID NO:18. In some embodiments, the anti-CD20 antibody or antigen-binding fragment thereof comprises a heavy chain variable region comprising the amino acid sequence of SEQ ID NO:17 and a light chain variable region comprising the amino acid sequence of SEQ ID NO:18. In some embodiments, the anti-CD20 antibody or antigen-binding fragment thereof is rituximab or a biosimilar thereof. In some embodiments, the anti-CD20 antibody or antigen-binding fragment thereof is rituximab. In some embodiments, the anti-CD20 antibody or antigen-binding fragment thereof is administered at a dose of 100 mg/m² to 500 mg/m² of the subject's body surface area. In some embodiments, the anti-CD20 antibody or antigen-binding fragment thereof is administered at a dose of about 375 mg/m² of the subject's body surface area. In some embodiments, the anti-CD20 antibody or antigen-binding fragment thereof is administered at a dose of 375 mg/m² of the subject's body surface area. In some embodiments, the anti-CD20 antibody or antigen-binding fragment thereof is administered at a dose of 500 mg to 2000 mg. In some embodiments, the anti-CD20 antibody or antigen-binding fragment thereof is administered at a dose of about 1400 mg. In some embodiments, the anti-CD20 antibody or antigen-binding fragment thereof is administered at a dose of 1400 mg. In some embodiments, the anti-CD20 antibody or antigen-binding fragment thereof is administered to the subject once about every 3 weeks. In some embodiments, the anti-CD20 antibody or antigen-binding fragment thereof is administered to the subject once every 3 weeks. In some embodiments, the anti-CD20 antibody or antigen-binding fragment thereof is administered to the subject on about day 1 of about a 21-day treatment cycle. In some embodiments, the anti-CD20 antibody or antigen-binding fragment thereof is administered to the subject on day 1 of a 21-day treatment cycle. In some embodiments, the anti-CD20 antibody or antigen-binding fragment thereof is administered by intravenous infusion. In some embodiments, the anti-CD20 antibody or antigen-binding fragment thereof is administered by subcutaneous injection. In some embodiments, the anti-CD20 antibody or antigen-binding fragment thereof is administered at a dose of about 375 mg/m² of the subject's body surface area by intravenous infusion on about day 1 of the first 21-day treatment cycle and is administered at a dose of about 1400 mg by subcutaneous injection on about day 1 of each 21-day treatment cycle thereafter. In some embodiments, the anti-CD20 antibody or antigen-binding fragment thereof is administered at a dose of 375 mg/m² of the subject's body surface area by intravenous infusion on day 1 of the first 21-day treatment cycle and is administered at a dose of 1400 mg by subcutaneous injection on day 1 of each 21-day treatment cycle thereafter. In some embodiments, the method further comprises the administration of granulocyte-colony stimulating factor (G-CSF) to the subject. In some embodiments, the G-CSF is administered 1 to 3 days after the administration of the anti-CD30 antibody-drug conjugate. In some embodiments, the G-CSF is selected from the group consisting of filgrastim, PEG-filgrastim, lenograstim, and tbo-filgrastim. In some embodiments, administering the lenalidomide, or salt or solvate thereof, and the antibody-drug conjugate that binds to CD30 to the subject results in a depletion of cancer cells by at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or about 100% compared to the amount of cancer cells before administering the lenalidomide, or salt or solvate thereof, and/or the antibody-drug conjugate that binds to CD30 to the subject. In some embodiments, one or more therapeutic effects in the subject is improved after administration of the lenalidomide, or salt or solvate thereof, and the antibody-drug conjugate that binds to CD30 relative to a baseline. In some embodiments, the one or more therapeutic effects is selected from the group consisting of: objective response rate, duration of response, time to response, progression free survival and overall survival. In some embodiments, the objective response rate is at least about 20%, at least about 25%, at least about 30%, at least about 35%, at least about 40%, at least about 45%, at least about 50%, at least about 60%, at least about 70%, or at least about 80%. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the lenalidomide, or salt or solvate thereof, and/or the antibody-drug conjugate that binds to CD30. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the lenalidomide, or salt or solvate thereof, and/or the antibody-drug conjugate that binds to CD30. In some embodiments, the duration of response to the lenalidomide, or salt or solvate thereof, and/or the antibody-drug conjugate that binds to CD30 is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the lenalidomide, or salt or solvate thereof, and/or the antibody-drug conjugate that binds to CD30. In some embodiments, the subject is a human.

Also provided herein is a pharmaceutical composition for the treatment of non Hodgkin lymphoma in a subject, the composition comprising lenalidomide, or salt or solvate thereof, and an antibody-drug conjugate that binds to CD30, wherein the antibody-drug conjugate comprises an anti-CD30 antibody or an antigen-binding fragment thereof conjugated to a monomethyl auristatin or a functional analog thereof or a functional derivative thereof, wherein the composition is for use in a method of any of the embodiments herein. In some embodiments, the pharmaceutical composition further comprises an anti-CD20 antibody or antigen-binding fragment thereof.

Also provided herein is a kit comprising lenalidomide, or salt or solvate thereof, and an antibody-drug conjugate that binds to CD30, wherein the antibody-drug conjugate comprises an anti-CD30 antibody or an antigen-binding fragment thereof conjugated to a monomethyl auristatin or a functional analog thereof or a functional derivative thereof, and instructions for using the kit in a method of any of the embodiments herein. In some embodiments, the kit further comprises an anti-CD20 antibody or antigen-binding fragment thereof.

### BRIEF DESCRIPTION OF THE DRAWINGS

FIG. 1A-1B is a series of graphs showing progression free survival (FIG. 1A) and overall survival (FIG. 1B) of subjects treated with brentuximab vedotin and lenalidomide.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

“CD30” or “TNFRSF8” refers to a receptor that is a member of the tumor necrosis factor receptor superfamily. CD30 is a transmembrane glycoprotein expressed on activated CD4⁺ and CD8⁺ T cells and B cells, and virally-infected lymphocytes. CD30 interacts with TRAF2 and TRAF3 to mediate signal transduction that leads to activation of NF-κB. CD30 acts as a positive regulator of apoptosis, and it has been shown to limit the proliferative potential of auto-reactive CD8 effector T cells. CD30 is also expressed by various forms of lymphoma, including Hodgkin lymphoma (CD30 is expressed by Reed-Sternberg cells) and non-Hodgkin lymphoma (e.g., diffuse large B-cell lymphoma (DLBCL), peripheral T-cell lymphoma (PTCL), and cutaneous T-cell lymphoma (CTCL).

The term “immunotherapy” refers to the treatment of a subject afflicted with, at risk of contracting, or suffering a recurrence of a disease by a method comprising inducing, enhancing, suppressing, or otherwise modifying an immune response.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N .Y. (1989)). Briefly, each heavy chain typically is comprised of a heavy chain variable region (abbreviated herein as V_{H} or V_{H}) and a heavy chain constant region (C_{H} or C_{H}). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or V_{L}) and a light chain constant region (C_{L} or C_{L}). The light chain constant region typically is comprised of one domain, C_{L}. The C_{L} can be of κ (kappa) or λ (lambda) isotype. The terms “constant domain” and “constant region” are used interchangeably herein. An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “variable region” or “variable domain” refers to the domain of an antibody heavy or light chain that is involved in binding the antibody to antigen. The variable regions of the heavy chain and light chain (V_{H} and V_{L}, respectively) of a native antibody may be further subdivided into regions of hypervariability (or hypervariable regions, which may be hypervariable in sequence and/or form of structurally defined loops), also termed complementarity-determining regions (CDRs), interspersed with regions that are more conserved, termed framework regions (FRs). The terms “complementarity determining regions” and “CDRs,” synonymous with “hypervariable regions” or “HVRs” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each VH and VL, three CDRs and four FRs are typically arranged from amino-terminus to carboxy-terminus in the following order: FR1, CDR1, FR2, CDR2, FR3, CDR3, FR4 (See also Chothia and Lesk J. Mot. Biol., 195, 901-917 (1987)).

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 min, at least about 45 min, at least about one hour (h), at least about two hours, at least about four hours, at least about eight hours, at least about 12 hours (h), about 24 hours or more, about 48 hours or more, about three, four, five, six, seven or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as C1q, the first component in the classical pathway of complement activation. An antibody may also be a bispecific antibody, diabody, multispecific antibody or similar molecule.

The term “monoclonal antibody” as used herein refers to a preparation of antibody molecules that are recombinantly produced with a single primary amino acid sequence. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

An “isolated antibody” refers to an antibody that is substantially free of other antibodies having different antigenic specificities (e.g., an isolated antibody that binds specifically to CD30 or CD20 is substantially free of antibodies that bind specifically to antigens other than CD30 or CD20, respectively). An isolated antibody that binds specifically to CD30 or CD20 can, however, have cross-reactivity to other antigens, such as CD30 or CD20 molecules from different species. Moreover, an isolated antibody can be substantially free of other cellular material and/or chemicals. In one embodiment, an isolated antibody includes an antibody conjugate attached to another agent (e.g., small molecule drug). In some embodiments, an isolated anti-CD30 antibody includes a conjugate of an anti-CD30 antibody with a small molecule drug (e.g., MMAE or MMAF).

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generic for different kinds of modifications of antibodies, and which is a well-known process for the skilled person. In particular, a chimeric antibody may be generated by using standard DNA techniques as described in Sambrook et aL, 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein. Chimeric monoclonal antibodies for therapeutic applications are developed to reduce antibody immunogenicity. They may typically contain non-human (e.g. murine) variable regions, which are specific for the antigen of interest, and human constant antibody heavy and light chain domains. The terms “variable region” or “variable domains” as used in the context of chimeric antibodies, refers to a region which comprises the CDRs and framework regions of both the heavy and light chains of the immunoglobulin.

An “anti-antigen antibody” refers to an antibody that binds to the antigen. For example, an anti-CD30 antibody is an antibody that binds to the antigen CD30. In another example, an anti-CD20 antibody is an antibody that binds to the antigen CD20.

An “antigen-binding portion” or “antigen-binding fragment” of an antibody refers to one or more fragments of an antibody that retain the ability to bind specifically to the antigen bound by the whole antibody. Examples of antibody fragments (e.g., antigen-binding fragment) include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

As used herein, the terms “binding”, “binds” or “specifically binds” in the context of the binding of an antibody to a pre-determined antigen typically is a binding with an affinity corresponding to a K_{D} of about 10⁻⁶ M or less, e.g. 10⁻⁷M or less, such as about 10⁻⁸M or less, such as about 10⁻⁹ M or less, about 10⁻¹⁰ M or less, or about 10⁻¹¹M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D} , and lower affinity is intended to refer to higher K_{D} .

The term “ADC” refers to an antibody-drug conjugate, which in the context of the present invention refers to an anti-CD30 antibody, which is coupled to a drug moiety (e.g., MMAE or MMAF) as described in the present application.

The abbreviations “vc” and “val-cit” refer to the dipeptide valine-citrulline.

The abbreviation “PAB” refers to the self-immolative spacer:

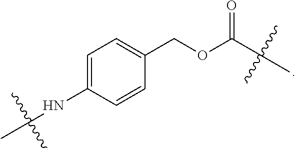 [SMILES: *Nc1ccc(COC(*)=O)cc1]

The abbreviation “MC” refers to the stretcher maleimidocaproyl:

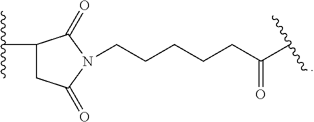 [SMILES: *C(=O)CCCCCN1C(=O)CC(*)C1=O]

The term “Ab-MC-vc-PAB-MMAE” refers to an antibody conjugated to the drug MMAE through a MC-vc-PAB linker.

The term “cAC10-MC-vc-PAB-MMAE” refers to a chimeric AC10 antibody conjugated to the drug MMAE through a MC-vc-PAB linker.

An “anti-CD30 vc-PAB-MMAE antibody-drug conjugate” refers to an anti-CD30 antibody conjugated to the drug MMAE via a linker comprising the dipeptide valine citrulline and the self-immolative spacer PAB as shown in Formula (I) of U.S. Pat. No. 9,211,319.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression can be observed and continue for a period of at least about 20 days, at least about 30 days, at least about 40 days, at least about 50 days, or at least about 60 days.

A therapeutically effective amount of a drug includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound that is lower than the usual or typical dose of the therapeutic compound when administered alone for the treatment of a hyperproliferative disease (e.g., cancer).

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease,

As used herein, “overall response rate” or “ORR” refers to the sum of complete response (CR) rate and partial response (PR) rate,

As used herein, “overall survival” or “OS” refers to the percentage of individuals in a. group who are likely to be alive after a particular duration of time.

The term “weight-based dose”, as referred to herein, means that a dose administered to a subject is calculated based on the weight of the subject. For example, when a subject with 60 kg body weight requires 1.2 mg/kg of an anti-CD30 antibody or an anti-CD30 antibody-drug conjugate, one can calculate and use the appropriate amount of the anti-CD30 antibody or anti-CD30 antibody-drug conjugate (i.e., 72 mg) for administration to said subject.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., the lenalidomide and/or anti-CD20 antibody). For example, a subject with 60 kg body weight and a subject with 100 kg body weight would receive the same dose (e.g., 20 mg of lenalidomide or e.g. 1400 mg of an anti-CD20 antibody).

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis -(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” or “administration” refer to the physical introduction of a therapeutic agent to a subject, using any of the various methods and delivery systems known to those skilled in the art. Exemplary routes of administration for the anti-CD30 antibody-drug conjugate and/or anti-CD20 antibody and/or lenalidomide include intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy (e.g., an anti-CD30 antibody-drug conjugate as described herein, an anti-CD20 antibody as described herein, and/or lenalidomide) or at the beginning of administration of the therapy. The baseline value can be compared to a reference value in order to determine the reduction or improvement of a symptom of a CD30-associated disease and/or CD20 associated disease contemplated herein (e.g., DLBCL). The terms “reference” or “reference value” used interchangeably herein can refer to a measurement or characterization of a symptom after administration of the therapy (e.g., an anti-CD30 antibody-drug conjugate as described herein, an anti-CD20 antibody as described herein, and/or lenalidomide). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that the anti-CD30 antibody-drug conjugate, anti-CD20 antibody, or lenalidomide is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days ±one day, i.e., every six days to every eight days. “Once about every two weeks” can include every fourteen days ±two days, i.e., every twelve days to every sixteen days. “Once about every three weeks” can include every twenty-one days ±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Combination Therapy

One aspect of the invention provides anti-CD30 antibody-drug conjugates that binds to CD30 for use in the treatment of non-Hodgkin lymphoma wherein the antibody-drug conjugate is for administration, or to be administered in combination with lenalidomide, or salt or solvate thereof, wherein the antibody-drug conjugate comprises an anti-CD30 antibody or an antigen-binding fragment thereof conjugated to a monomethyl auristatin or a functional analog thereof or a functional derivative thereof. In some embodiments, the anti-CD30 antibody or antigen-binding fragment thereof comprises the complementary determining regions (CDRs) of brentuximab, or a biosimilar thereof. In some embodiments, the anti-CD30 antibody or antigen-binding fragment thereof comprises the complementary determining regions (CDRs) of brentuximab. In some embodiments, the anti-CD30 antibody or antigen-binding fragment thereof comprises the heavy chain variable region and the light chain variable region of brenutximab, or a biosimilar thereof. In some embodiments, the anti-CD30 antibody or antigen-binding fragment thereof comprises the heavy chain variable region and the light chain variable region of brentuximab. In some embodiments, the anti-CD30 antibody is brentuximab or a biosimilar thereof. In some embodiments, the anti-CD30 antibody is brentuximab. In some embodiments, the antibody-drug conjugate is brentuximab vedotin.or a biosimilar thereof. In some embodiments, the antibody-drug conjugate is brentuximab vedotin. In some embodiments, the treatment further comprises the administration of an anti-CD20 antibody or an antigen-binding fragment thereof. In some embodiments, the anti-CD20 antibody or antigen-binding fragment thereof comprises the complementary determining regions (CDRs) of rituximab, or a biosimilar thereof. In some embodiments, the anti-CD20 antibody or antigen-binding fragment thereof comprises the complementary determining regions (CDRs) of rituximab. In some embodiments, the anti-CD20 antibody or antigen-binding fragment thereof comprises the heavy chain variable region and the light chain variable region of rituximab, or a biosimilar thereof. In some embodiments, the anti-CD20 antibody or antigen-binding fragment thereof comprises the heavy chain variable region and the light chain variable region of rituximab. In some embodiments, the anti-CD20 antibody is rituximab. In some embodiments, the non-Hodgkin lymphoma is an advanced stage non-Hodgkin lymphoma. In some embodiments, the advanced non-Hodgkin lymphoma is a stage 3 or stage 4 non-Hodgkin lymphoma. In some embodiments, the advanced non-Hodgkin lymphoma is a metastatic non-Hodgkin lymphoma. In some embodiments, the non-Hodgkin lymphoma is recurrent non-Hodgkin lymphoma. In some embodiments, the non-Hodgkin lymphoma is diffuse large B-cell lymphoma (DLBCL). In some embodiments, the DLBCL is relapsed DLBCL. In some embodiments, the DLBCL is recurrent DLBCL. In some embodiments, the DLBCL is germinal-center B-cell like (GCB). In some embodiments, the DLBCL is non-GCB.

### A. Anti-CD30 Antibodies and Antibody-Drug Conjugates

I. Anti-CD30 Antibody

In one aspect, the therapy of the present disclosure utilizes an anti-CD30 antibody or an antigen-binding fragment thereof. CD30 receptors are members of the tumor necrosis factor receptor superfamily involved in limiting the proliferative potential of autoreactive CD8 effector T cells. Antibodies targeting CD30 can potentially be either agonists or antagonists of these CD30 mediated activities. In some embodiments, the anti-CD30 antibody is conjugated to a therapeutic agent (e.g., an anti-CD30 antibody-drug conjugate).

Murine anti-CD30 mAbs known in the art have been generated by immunization of mice with Hodgkin's disease (HD) cell lines or purified CD30 antigen. AC10, originally termed C10 (Bowen et al., 1993, J. Immunol. 151:5896 5906), is distinct in that this anti-CD30 mAb that was prepared against a hum an NK-like cell line, YT (Bowen et al., 1993, J. Immunol. 151:5896 5906). Initially, the signaling activity of this mAb was evidenced by the down regulation of the cell surface expression of CD28 and CD45 molecules, the up regulation of cell surface CD25 expression and the induction of homotypic adhesion following binding of C10 to YT cells. Sequences of the AC10 antibody are set out in SEQ ID NO: 1-16. See also U.S. Pat. No. 7,090,843, incorporated herein by reference.

Generally, anti-CD30 antibodies of the disclosure bind CD30, e.g., human CD30, and exert cytostatic and cytotoxic effects on cells expressing CD30. Anti-CD30 antibodies of the disclosure are preferably monoclonal, and may be multispecific, human, humanized or chimeric antibodies, single chain antibodies, Fab fragments, F(ab′) fragments, fragments produced by a Fab expression library, and CD30 binding fragments of any of the above. In some embodiments, the anti-CD30 antibodies of the disclosure specifically bind CD30. The immunoglobulin molecules of the disclosure can be of any type (e.g., IgG, IgE, IgM, IgD, IgA and IgY), class (e.g., IgG1, IgG2, IgG3, IgG4, IgAl and IgA2) or subclass of immunoglobulin molecule.

In certain embodiments of the disclosure, the anti-CD30 antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a VL or VH domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-CD30 antibodies or antigen-binding fragments thereof are human, murine (e.g., mouse and rat), donkey, sheep, rabbit, goat, guinea pig, camelid, horse, or chicken.

The anti-CD30 antibodies of the present disclosure may be monospecific, bispecific, trispecific or of greater multi specificity. Multispecific antibodies may be specific for different epitopes of CD30 or may be specific for both CD30 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-CD30 antibodies of the present disclosure may be described or specified in terms of the particular CDRs they comprise. In certain embodiments antibodies of the disclosure comprise one or more CDRs of AC10. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” “J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementarity determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

The disclosure encompasses an antibody or derivative thereof comprising a heavy or light chain variable domain, said variable domain comprising (a) a set of three CDRs, in which said set of CDRs are from monoclonal antibody AC10, and (b) a set of four framework regions, in which said set of framework regions differs from the set of framework regions in monoclonal antibody AC10, and in which said antibody or derivative thereof immunospecifically binds CD30.

In one aspect, the anti-CD30 antibody is AC10. In some embodiments, the anti-CD30 antibody is cAC10. cAC10 is a chimeric IgG1 monoclonal antibody that specifically binds CD30. cAC10 induces growth arrest of CD30⁺ cell lines in vitro and has pronounced antitumor activity in severe combined immunodeficiency (SCID) mouse xenograft models of Hodgkin disease. See Francisco et al., Blood 102(4):1458-64 (2003). AC10 antibody and cAC10 antibody are described in U.S. Pat. No. 9,211,319 and 7,090,843.

In one aspect, anti-CD30 antibodies that compete with AC10 antibody and/or cAC10 antibody binding to CD30 are provided. Anti-CD30 antibodies that bind to the same epitope as AC10 antibody and cAC10 antibody are also provided.

In one aspect, provided herein is an anti-CD30 antibody comprising 1, 2, 3, 4, 5, or 6 of the CDR sequences of the AC10 antibody. In one aspect, provided herein is an anti-CD30 antibody comprising 1, 2, 3, 4, 5, or 6 of the CDR sequences of the cAC10 antibody. In some embodiments, the CDR is a Kabat CDR or a Chothia CDR.

In one aspect, provided herein is an anti-CD30 antibody comprising a heavy chain variable region and a light chain variable region, wherein the heavy chain variable region comprises (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3; and/or wherein the light chain variable region comprises (i) CDR-L1 comprising the amino acid sequence of SEQ ID NO:4, (ii) CDR-L2 comprising the amino acid sequence of SEQ ID NO:5, and (iii) CDR-L3 comprising the amino acid sequence of SEQ ID NO:6.

An anti-CD30 antibody described herein may comprise any suitable framework variable domain sequence, provided that the antibody retains the ability to bind CD30 (e.g., human CD30). As used herein, heavy chain framework regions are designated “HC-FR1-FR4,” and light chain framework regions are designated “LC-FR1-FR4.” In some embodiments, the anti-CD30 antibody comprises a heavy chain variable domain framework sequence of SEQ ID NO:9, 10, 11, and 12 (HC-FR1, HC-FR2, HC-FR3, and HC-FR4, respectively). In some embodiments, the anti-CD30 antibody comprises a light chain variable domain framework sequence of SEQ ID NO:13, 14, 15, and 16 (LC-FR1, LC-FR2, LC-FR3, and LC-FR4, respectively).

In one embodiment, an anti-CD30 antibody comprises a heavy chain variable domain comprising a framework sequence and hypervariable regions, wherein the framework sequence comprises the HC-FR1-HC-FR4 amino acid sequences of SEQ ID NO:9 (HC-FR1), SEQ ID NO:10 (HC-FR2), SEQ ID NO:11 (HC-FR3), and SEQ ID NO:12 (HC-FR4), respectively; the CDR-H1 comprises the amino acid sequence of SEQ ID NO:1; the CDR-H2 comprises the amino acid sequence of SEQ ID NO:2; and the CDR-H3 comprises the amino acid sequence of SEQ ID NO:3.

In one embodiment, an anti-CD30 antibody comprises a light chain variable domain comprising a framework sequence and hypervariable regions, wherein the framework sequence comprises the LC-FR1-LC-FR4 amino acid sequences of SEQ ID NO:13 (LC-FR1), SEQ ID NO:14 (LC-FR2), SEQ ID NO:15 (LC-FR3), and SEQ ID NO:16 (LC-FR4), respectively; the CDR-L1 comprises the amino acid sequence of SEQ ID NO:4; the CDR-L2 comprises the amino acid sequence of SEQ ID NO:5; and the CDR-L3 comprises the amino acid sequence of SEQ ID NO:6.

In some embodiments of the anti-CD30 antibodies described herein, the heavy chain variable domain comprises the amino acid sequence of QIQLQQSGPEVVKPGASVKISCKASGYTFTDYYITWVKQKPGQGLEWIGWIYPGSGNTK YNEKFKGKATLTVDTSSSTAFMQLSSLTSEDTAVYFCANYGNYWFAYWGQGTQVTVS A (SEQ ID NO:7) and the light chain variable domain comprises the amino acid sequence of DIVLTQSPASLAVSLGQRATISCKASQSVDFDGDSYMNWYQQKPGQPPKVLIYAASNLE SGIPARFSGSGSGTDFTLNIHIPVEEEDAATYYCQQSNEDPWTFGGGTKLEIK (SEQ ID NO:8).

In some embodiments of the anti-CD30 antibodies described herein, the heavy chain CDR sequences comprise the following:

a) CDR-H1
(DYYIT (SEQ ID NO: 1));
b) CDR-H2
(WIYPGSGNTKYNEKFKG (SEQ ID NO: 2));
and
c) CDR-H3
(YGNYWFAY (SEQ ID NO: 3)).

In some embodiments of the anti-CD30 antibodies described herein, the heavy chain FR sequences comprise the following:

a) HC-FR1
(QIQLQQSGPEVVKPGASVKISCKASGYTFT (SEQ ID NO: 9));
b) HC-FR2
(WVKQKPGQGLEWIG (SEQ ID NO: 10));
c) HC-FR3
(KATLTVDTSSSTAFMQLSSLTSEDTAVYFCAN (SEQ ID NO:
11));
and
d) HC-FR4
(WGQGTQVTVSA (SEQ ID NO: 12)).

In some embodiments of the anti-CD30 antibodies described herein, the light chain CDR sequences comprise the following:

a) CDR-L1
(KASQSVDFDGDSYMN (SEQ ID NO: 4));
b) CDR-L2
(AASNLES (SEQ ID NO: 5));
and
c) CDR-L3
(QQSNEDPWT (SEQ ID NO: 6)).

In some embodiments of the anti-CD30 antibodies described herein, the light chain FR sequences comprise the following:

a) LC-FR1
(DIVLTQSPASLAVSLGQRATISC (SEQ ID NO: 13));
b) LC-FR2
(WYQQKPGQPPKVLIY (SEQ ID NO: 14));
c) LC-FR3
(GIPARFSGSGSGTDFTLNIHPVEEEDAATYYC (SEQ ID NO:
15));
and
d) LC-FR4
(FGGGTKLEIK (SEQ ID NO: 16)).

In some embodiments, provided herein is an anti-CD30 antibody that binds to CD30 (e.g., human CD30), wherein the antibody comprises a heavy chain variable region and a light chain variable region, wherein the antibody comprises:
(a) heavy chain variable domain comprising:
(1) an HC-FRI comprising the amino acid sequence of SEQ ID NO:9;
(2) an CDR-H1 comprising the amino acid sequence of SEQ ID NO:1;
(3) an HC-FR2 comprising the amino acid sequence of SEQ ID NO:10;
(4) an CDR-H2 comprising the amino acid sequence of SEQ ID NO:2;
(5) an HC-FR3 comprising the amino acid sequence of SEQ ID NO:11;
(6) an CDR-H3 comprising the amino acid sequence of SEQ ID NO:3; and
(7) an HC-FR4 comprising the amino acid sequence of SEQ ID NO:12, and/or
(b) a light chain variable domain comprising:
(1) an LC-FR1 comprising the amino acid sequence of SEQ ID NO:13;
(2) an CDR-L1 comprising the amino acid sequence of SEQ ID NO:4;
(3) an LC-FR2 comprising the amino acid sequence of SEQ ID NO:14;
(4) an CDR-L2 comprising the amino acid sequence of SEQ ID NO:5;
(5) an LC-FR3 comprising the amino acid sequence of SEQ ID NO:15;
(6) an CDR-L3 comprising the amino acid sequence of SEQ ID NO:6; and
(7) an LC-FR4 comprising the amino acid sequence of SEQ ID NO:16.

In one aspect, provided herein is an anti-CD30 antibody comprising a heavy chain variable domain comprising the amino acid sequence of SEQ ID NO:7 and/or comprising a light chain variable domain comprising the amino acid sequence of SEQ ID NO:8.

In some embodiments, provided herein is an anti-CD30 antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7 contains substitutions (e.g., conservative substitutions), insertions, or deletions relative to the reference sequence and retains the ability to bind to a CD30 (e.g., human CD30). In certain embodiments, a total of 1 to 10 amino acids have been substituted, inserted and/or deleted in SEQ ID NO:7. In certain embodiments, substitutions, insertions, or deletions (e.g., 1, 2, 3, 4, or 5 amino acids) occur in regions outside the CDR s (i.e., in the FRs). In some embodiments, the anti-CD30 antibody comprises a heavy chain variable domain sequence of SEQ ID NO:7 including post-translational modifications of that sequence. In a particular embodiment, the heavy chain variable domain comprises one, two or three CDRs selected from: (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3.

In some embodiments, provided herein is an anti-CD30 antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8 contains substitutions (e.g., conservative substitutions), insertions, or deletions relative to the reference sequence and retains the ability to bind to a CD30 (e.g., human CD30). In certain embodiments, a total of 1 to 10 amino acids have been substituted, inserted and/or deleted in SEQ ID NO:8. In certain embodiments, substitutions, insertions, or deletions (e.g., 1, 2, 3, 4, or 5 amino acids) occur in regions outside the CDR s (i.e., in the FRs). In some embodiments, the anti-CD30 antibody comprises a light chain variable domain sequence of SEQ ID NO:8 including post-translational modifications of that sequence. In a particular embodiment, the light chain variable domain comprises one, two or three CDRs selected from: (a) CDR-H1 comprising the amino acid sequence of SEQ ID NO:4, (b) CDR-H2 comprising the amino acid sequence of SEQ ID NO:5, and (c) CDR-H3 comprising the amino acid sequence of SEQ ID NO:6.

In some embodiments, the anti-CD30 antibody comprises a heavy chain variable domain as in any of the embodiments provided above, and a light chain variable domain as in any of the embodiments provided above. In one embodiment, the antibody comprises the heavy chain variable domain sequence of SEQ ID NO:7 and the light chain variable domain sequence of SEQ ID NO:8, including post-translational modifications of those sequences.

In some embodiments, the anti-CD30 antibody of the anti-CD30 antibody-drug conjugate comprises: i) a heavy chain CDR1 set out in SEQ ID NO: 1, a heavy chain CDR2 set out in SEQ ID NO: 2, a heavy chain CDR3 set out in SEQ ID NO: 3; and ii) a light chain CDR1 set out in SEQ ID NO: 4, a light chain CDR2 set out in SEQ ID NO: 5, and a light chain CDR3 set out in SEQ ID NO: 6.

In some embodiments, the anti-CD30 antibody of the anti-CD30 antibody-drug conjugate comprises: i) an amino acid sequence at least 85% identical to a heavy chain variable region set out in SEQ ID NO: 7, and ii) an amino acid sequence at least 85% identical to a light chain variable region set out in SEQ ID NO: 8.

In some embodiments, the anti-CD30 antibody of the anti-CD30 antibody-drug conjugate is a monoclonal antibody.

In some embodiments, the anti-CD30 antibody of the anti-CD30 antibody-drug conjugate is a chimeric AC10 antibody.

In some embodiments, the anti-CD30 antibody of the anti-CD30 antibody-drug conjugate is brentuximab or a biosimilar thereof. In some embodiments, the anti-CD30 antibody of the anti-CD30 antibody-drug conjugate is brentuximab.

Antibodies of the present invention may also be described or specified in terms of their binding affinity to CD30. Preferred binding affinities include those with a dissociation constant or Kd less than 5×10² M, 10⁻² M, 5×10⁻³ M, 10⁻³ M, 5×10⁻⁴M, 10⁻⁴ M, 5×10⁻⁵M, 10⁻⁵ M, 5×10⁻⁶ M, 10⁻⁶M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻² M, 5×10⁻¹³ M, 10⁻¹³ M, 5×10⁻¹⁴ M, 10⁻¹⁴ M, 5×10−15 M, or 10⁻¹⁵ M.

There are five classes of immunoglobulins: IgA, IgD, IgE, IgG and IgM, having heavy chains designated α, δ, ε, γ and μ, respectively. The y and a classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

In one aspect of the invention, polynucleotides encoding anti-CD30 antibodies, such as those anti-CD30 antibodies described herein, are provided. In certain embodiments, vectors comprising polynucleotides encoding anti-CD30 antibodies as described herein are provided. In certain embodiments, host cells comprising such vectors are provided. In another aspect of the invention, compositions comprising anti-CD30 antibodies described herein or polynucleotides encoding anti-CD30 antibodies described herein are provided.

The antibodies also include derivatives that are modified, i.e., by the covalent attachment of any type of molecule to the antibody such that covalent attachment does not prevent the antibody from binding to CD30 or from exerting a cytostatic or cytotoxic effect on HD cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

II. Antibody-Drug Conjugate Structure

In some embodiments, the anti-CD30 antibody is conjugated to a therapeutic agent (e.g., an anti-CD30 antibody-drug conjugate). In some embodiments, the therapeutic agent comprises an anti-neoplastic agent (e.g., an anti-mitotic agent). In certain embodiments, the therapeutic agent is an auristatin. In certain embodiments, the therapeutic agent is selected from the group consisting of monomethyl auristatin E (MMAE), monomethyl auristatin F (MMAF), auristatin drug analogues, cantansinoids, maytansinoids (e.g., maytansine; DMs), dolastatins, cryptophycin, duocarmycin, duocarmycin derivatives, esperamicin, calicheamicin, pyrolobenodiazepine (PBD), and any combination thereof. In one particular embodiment, the anti-CD30 antibody is conjugated to MMAE. The antibody can be conjugated to at least one, at least two, at least three, at least four, at least five, at least six, at least seven, at least eight, at least nine, or at least ten molecules of the therapeutic agent (e.g., MMAE). In one embodiment, the anti-CD30 antibody is conjugated to four molecules of the therapeutic agent, e.g., four molecules of MMAE. In one particular embodiment, the anti-CD30 antibody is conjugated to MMAF. The antibody can be conjugated to at least one, at least two, at least three, at least four, at least five, at least six, at least seven, at least eight, at least nine, or at least ten molecules of the therapeutic agent (e.g., MMAF). In one embodiment, the anti-CD30 antibody is conjugated to four molecules of the therapeutic agent, e.g., four molecules of MMAF.

In one embodiment, the auristatin is monomethyl auristatin E (MMAE):

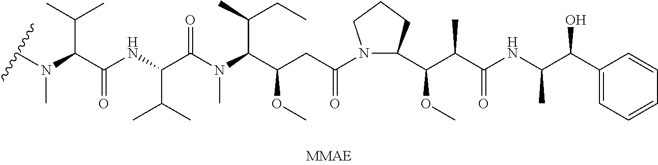 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates the attachment site for the linker.

In one embodiment, the auristatin is monomethyl auristatin F (MMAF):

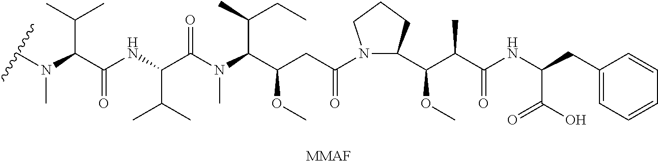

wherein the wavy line indicates the attachment site for the linker.

In some embodiments, the anti-CD30 antibody-drug conjugate further comprises a linker between the therapeutic agent and the antibody. In some embodiments, the linker comprises one or more naturally occurring amino acids, one or more non-naturally occurring (e.g., synthetic) amino acids, a chemical linker, or any combination thereof. In certain embodiments, the linker is a cleavable linker, e.g., a protease cleavable linker. In certain embodiments, the linker is specifically cleaved upon uptake by a target cell, e.g., upon uptake by a cell expressing CD30. In certain embodiments, the linker is a cleavable peptide linker having the formula: “-MC-vc-PAB-” or “-MC-val-cit-PAB-”, wherein “MC” refers to the stretcher maleimidocaproyl having the following structure:

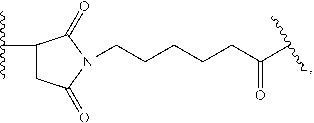 [SMILES: *C(=O)CCCCCN1C(=O)CC(*)C1=O]

“vc” and “val-cit” refer to the dipeptide valine-citrulline, and PAB refers to a self-immolative spacer having the following structure:

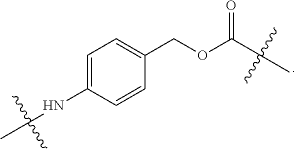 [SMILES: *Nc1ccc(COC(*)=O)cc1]

In some embodiments, cleavage of the linker activates a cytotoxic activity of the therapeutic agent. In certain embodiments, the linker is a non-cleavable linker. In certain embodiments, the non-cleavable linker has the formula: “-MC-”, wherein “MC” refers to the stretcher maleimidocaproyl having the following structure:

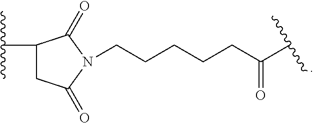 [SMILES: *C(=O)CCCCCN1C(=O)CC(*)C1=O]

In some embodiments, the antibody-drug conjugates comprises an anti-CD30 antibody, covalently linked to MMAE through a vc-PAB linker. In some embodiments, the antibody-drug conjugate is delivered to the subject as a pharmaceutical composition. In some embodiments, the CD30 antibody-drug conjugates contemplated herein are as described in U.S. Pat. No. 9,211,319, herein incorporated by reference.

In one embodiment, the anti-CD30 antibody-drug conjugate comprises brentuximab vedotin. In one particular embodiment, the anti-CD30 antibody-drug conjugate is brentuximab vedotin or a biosimilar thereof. In one particular embodiment, the anti-CD30 antibody-drug conjugate is brentuximab vedotin. Brentuximab vedotin (BV; also known as “ADCETRIS®”) is a CD30-directed antibody-drug conjugate (ADC) comprising a chimeric anti-CD30 antibody (cAC10), a therapeutic agent (MMAE), and a protease-cleavable linker between the cAC10 and the MMAE, as shown in the following structure:

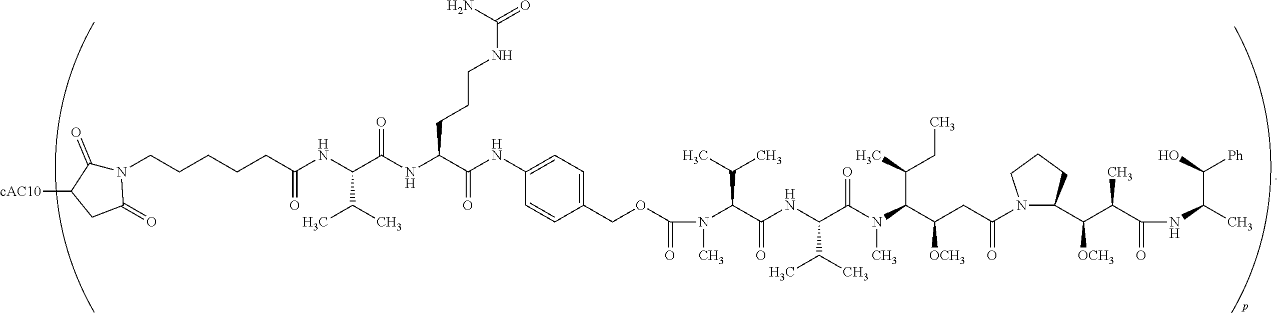

The drug to antibody ratio or drug loading is represented by “p” in the structure of brentuximab vedotin and ranges in integer values from 1 to 8. The average drug loading of brentuximab vedotin in a pharmaceutical composition is about 4. ADCETRIS® is approved by the FDA for treatment of patients with Hodgkin lymphoma after failure of autologous stem cell transplant (ASCT) or after failure of at least two prior multi-agent chemotherapy regimens in patients who are not ASCT candidates and for the treatment of patients with systemic anaplastic large cell lymphoma after failure of at least one prior multi-agent chemotherapy regimen.

In one embodiment, the anti-CD30 antibody is an anti-CD30 antibody or antigen-binding fragment thereof that binds to the same epitope as cAC10, e.g., the same epitope as brentuximab vedotin. In certain embodiments, the anti-CD30 antibody is an antibody that has the same CDRs as cAC10, e.g., the same CDRs as brentuximab vedotin. Antibodies that bind to the same epitope are expected to have functional properties very similar to those of cAC10 by virtue of their binding to the same epitope region of CD30. These antibodies can be readily identified based on their ability to, for example, cross-compete with cAC10 in standard CD30 binding assays such as Biacore analysis, ELISA assays, or flow cytometry.

In certain embodiments, the antibodies that cross-compete for binding to human CD30 with, or bind to the same epitope region of human CD30 as cAC10 are monoclonal antibodies. For administration to human subjects, these cross-competing antibodies can be chimeric antibodies, or can be humanized or human antibodies. Such chimeric, humanized, or human monoclonal antibodies can be prepared and isolated by methods well known in the art. Anti-CD30 antibodies usable in the methods of the disclosed disclosure also include antigen-binding portions of the above antibodies.

In other embodiments, the anti-CD30 antibody or antigen-binding portion thereof is a chimeric, humanized, or human monoclonal antibody or a portion thereof. In certain embodiments for treating a human subject, the antibody is a humanized antibody. In other embodiments for treating a human subject, the antibody is a human antibody. Antibodies of an IgG1, IgG2, IgG3, or IgG4 isotype can be used.

In one embodiment, the antibody-drug conjugate is brentuximab vedotin or a biosimilar thereof. In one embodiment, the antibody-drug conjugate is brentuximab vedotin.

### B. Lenalidomide

In one aspect, the therapy of the present disclosure utilizes lenalidomide, or salt or solvate thereof. Lenalidomide is also known as REVLIMIDO and (RS)-3-(4-Amino-1-oxo-1,3-dihydro-2H-isoindol-2-yl)piperidine-2,6-dione.

Lenalidomide is a racemic mixture of the following two structures:

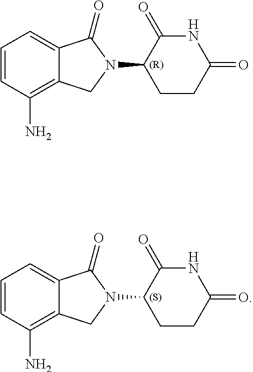

C. Anti-CD20 Antibody

In one aspect, the therapy of the present disclosure utilizes an anti-CD20 antibody or an antigen-binding fragment thereof. The CD20 antigen (also called human B-lymphocyte-restricted differentiation antigen, Bp35) is a hydrophobic transrnembrane protein with a molecular weight of approximately 35 kD located on pre-B and mature B lymphocytes. The antigen is also expressed on greater than 90% of B cell non-Hodgkin's lymphomas (NHL), but is not found on hematopoietic stem cells, pro-B cells, normal plasma cells or other normal tissues. Given the expression of CD20 in B cell lymphomas, this antigen has been a useful therapeutic target to treat such lymphomas.

In some embodiments, the anti-CD20 antibody is one described in U.S. Pat. Nos. 5,736,137 and 5,776,456. In some embodiments the anti-CD20 antibody is rituximab. Rituximab is a monoclonal, chimeric murine/human monoclonal IgG1 antibody. Rituximab is also known as RITUXAN® and MABTHERA®. In some embodiments, the anti-CD20 antibody is a biosimilar of rituximab, such as RIXATHON®, RUXIENCE® or TRUXIMA®.

In some embodiments, the anti-CD20 antibody is the antibody referred to as “C2B8” in U.S. Pat. Nos. 5,736,137 and 5,776,456.

Anti-CD20 antibodies of the disclosure are preferably monoclonal, and may be multispecific, human, humanized or chimeric antibodies, single chain antibodies, Fab fragments, F(ab′) fragments, fragments produced by a Fab expression library, and CD20 binding fragments of any of the above. In some embodiments, the anti-CD20 antibodies of the disclosure specifically bind CD20. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the anti-CD20 antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a VL or VH domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-CD20 antibodies or antigen-binding fragments thereof are human, murine (e.g., mouse and rat), donkey, sheep, rabbit, goat, guinea pig, camelid, horse, or chicken.

The anti-CD20 antibodies of the present disclosure may be monospecific, bispecific, trispecific or of greater multi specificity. Multispecific antibodies may be specific for different epitopes of CD20 or may be specific for both CD20 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-CD20 antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” “J. Mol. Biol. 262, 732-745.” (“Contact” numbering scheme); Lefranc NIP et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pliickthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementarity determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

CDR sequences of the anti-CD20 antibodies described herein are according to the Kabat numbering scheme as described in Kabat et al. (1991), “Sequences of Proteins of Immunological Interest,” 5th Ed. Public Health Service, National Institutes of Health, Bethesda, MD, unless specified otherwise.

In one aspect, provided herein is an anti-CD20 antibody comprising a heavy chain variable region comprising the three CDRs of SEQ ID NO:17 and a light chain variable region comprising the three CDRs of SEQ ID NO:18, wherein the CDRs of the anti-CD20 antibody are defined by the Kabat numbering scheme. In some embodiments, the anti-CD20 antibody further comprises an Fc domain.

An anti-CD20 antibody described herein may comprise any suitable framework variable domain sequence, provided that the antibody retains the ability to bind CD20 (e.g., human CD20). As used herein, heavy chain framework regions are designated “HC-FR1-FR4,” and light chain framework regions are designated “LC-FR1-FR4.”

In some embodiments of the anti-CD20 antibodies described herein, the heavy chain variable domain comprises the amino acid sequence of QVQLQQPGAELVKPGASVKMSCKASGYTFTSYNMHWVKQTPGRGLEWIGAIYPGNGD TSYNQKFKGKATLTADKSSSTAYMQLSSLTSEDSAVYYCARSTYYGGDWYFNVWGAG TTVTVS (SEQ ID NO:17) and the light chain variable domain comprises the amino acid sequence of QIVLSQSPAILSASPGEKVTMTCRASSSVSYIHWFQQKPGSSPKPWIYATSNLASGVPVRF SGSGSGTSYSLTISRVEAEDAATYYCQQWTSNPPTFGGGTKLEIK (SEQ ID NO:18).

In one aspect, provided herein is an anti-CD20 antibody comprising a heavy chain variable domain comprising the amino acid sequence of SEQ ID NO:17 or comprising a light chain variable domain comprising the amino acid sequence of SEQ ID NO:18. In one aspect, provided herein is an anti-CD20 antibody comprising a heavy chain variable domain comprising the amino acid sequence of SEQ ID NO:17 and comprising a light chain variable domain comprising the amino acid sequence of SEQ ID NO:18.

In some embodiments, provided herein is an anti-CD20 antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:17. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:17 contains substitutions (e.g., conservative substitutions), insertions, or deletions relative to the reference sequence and retains the ability to bind to a CD20 (e.g., human CD20). In certain embodiments, a total of 1 to 10 amino acids have been substituted, inserted and/or deleted in SEQ ID NO:17. In certain embodiments, substitutions, insertions, or deletions (e.g., 1, 2, 3, 4, or 5 amino acids) occur in regions outside the CDRs (i.e., in the FRs). In some embodiments, the anti-CD20 antibody comprises a heavy chain variable domain sequence of SEQ ID NO:17 including post-translational modifications of that sequence.

In some embodiments, provided herein is an anti-CD20 antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:18. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:18 contains substitutions (e.g., conservative substitutions), insertions, or deletions relative to the reference sequence and retains the ability to bind to a CD20 (e.g., human CD20). In certain embodiments, a total of 1 to 10 amino acids have been substituted, inserted and/or deleted in SEQ ID NO:18. In certain embodiments, substitutions, insertions, or deletions (e.g., 1, 2, 3, 4, or 5 amino acids) occur in regions outside the CDRs (i.e., in the FRs). In some embodiments, the anti-CD20 antibody comprises a light chain variable domain sequence of SEQ ID NO:18 including post-translational modifications of that sequence.

In some embodiments, provided herein is an anti-CD20 antibody comprising a heavy chain comprising an amino acid sequence having at least 85%, 86%, 87%, 88%, 89%, 90%, 91%, 92%, 93%, 94%, 95%, 96%, 97%, 98%, or 99% sequence identity to the amino acid sequence QVQLQQPGAELVKPGASVKMS CKAS GYTFTSYNMHWVKQTPGRGLEWIGAIYPGNGD TSYNQKFKGKATLTADKS S STAYMQLS SLTSEDSAVYYCARSTYYGGDWYFNVWGAG TTVTVSAASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKAEPKSCDKTHTCPPCP APELLGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPAPIEKTISKAKGQPREPQ VYTLPPSRDELTKNQVSLTCLVKGFYPSDIAVEWESNGQPENNYKTTPPVLDSDGSFFLY SKLTVDKSRWQQGNVFSCSVMHEALHNHYTQKSLSLSPGK (SEQ ID NO:19). In certain embodiments, a heavy chain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:19 contains substitutions (e.g., conservative substitutions), insertions, or deletions relative to the reference sequence and retains the ability to bind to a CD20 (e.g., human CD20). In certain embodiments, a total of 1 to 10 amino acids have been substituted, inserted and/or deleted in SEQ ID NO:19. In certain embodiments, substitutions, insertions, or deletions (e.g., 1, 2, 3, 4, or 5 amino acids) occur in regions outside the CDRs (i.e., in the FRs). In some embodiments, the anti-CD20 antibody comprises a heavy chain sequence of SEQ ID NO:19 including post-translational modifications of that sequence.

In some embodiments, provided herein is an anti-CD20 antibody comprising a light chain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of QIVLSQSPAILSASPGEKVTMTCRASSSVSYIHWFQQKPGS SPKPWIYATSNLASGVPVRF SGSGSGTSYSLTISRVEAEDAATYYCQQWTSNPPTFGGGTKLEIKRTVAAPSVFIFPPSDE QLKSGTASVVCLLNNFYPREAKVQWKVDNALQSGNSQESVTEQDSKDSTYSLSSTLTLS KADYEKHKVYACEVTHQGLSSPVTKSFNRGEC (SEQ ID NO:20). In certain embodiments, a light chain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:20 contains substitutions (e.g., conservative substitutions), insertions, or deletions relative to the reference sequence and retains the ability to bind to a CD20 (e.g., human CD20). In certain embodiments, a total of 1 to 10 amino acids have been substituted, inserted and/or deleted in SEQ ID NO:20. In certain embodiments, substitutions, insertions, or deletions (e.g., 1, 2, 3, 4, or 5 amino acids) occur in regions outside the CDRs (i.e., in the FRs). In some embodiments, the anti-CD20 antibody comprises a light chain sequence of SEQ ID NO:20 including post-translational modifications of that sequence.

In some embodiments, the anti-CD20 antibody comprises a heavy chain variable domain as in any of the embodiments provided above, and a light chain variable domain as in any of the embodiments provided above. In one embodiment, the antibody comprises the heavy chain variable domain sequence of SEQ ID NO:17 and the light chain variable domain sequence of SEQ ID NO:18, including post-translational modifications of those sequences.

In some embodiments, the anti-CD20 antibody comprises: i) an amino acid sequence having at least 85% sequence identity to a heavy chain variable region comprising the amino acid sequence of SEQ ID NO:17, and ii) an amino acid sequence having at least 85% sequence identity to a light chain variable region comprising the amino acid sequence of SEQ ID NO:18.

In some embodiments, the anti-CD20 antibody is a monoclonal antibody.

In some embodiments, the anti-CD20 antibody comprises a heavy chain variable region comprising the three CDRs or a light chain variable region comprising the three CDRs of an anti-CD20 antibody described in U.S. Pat. No. 5,736,137 or 5,776,456. In some embodiments, the anti-CD20 antibody comprises a heavy chain variable region comprising the three CDRs and a light chain variable region comprising the three CDRs of an anti-CD20 antibody described in U.S. Pat. No. 5,736,137 or 5,776,456. In some embodiments, the CDRs are defined by the Kabat numbering scheme.

In some embodiments, the anti-CD20 antibody comprises a heavy chain variable region or a light chain variable region of an anti-CD20 antibody described in U.S. Pat. No. 5,736,137 or 5,776,456. In some embodiments, the anti-CD20 antibody comprises a heavy chain variable region and a light chain variable region of an anti-CD20 antibody described in U.S. Pat. No. 5,736,137 or 5,776,456.

In some embodiments, the anti-CD20 antibody is an anti-CD20 antibody, such as an anti-CD20 antibody as described U.S. Pat. No. 5,736,137 or 5,776,456.

In some embodiments, the anti-CD20 antibody comprises a heavy chain variable region comprising the three CDRs or a light chain variable region comprising the three CDRs of the anti-CD20 antibody rituximab, or a biosimilar thereof. In some embodiments, the anti-CD20 antibody comprises a heavy chain variable region comprising the three CDRs and a light chain variable region comprising the three CDRs of the anti-CD20 antibody rituximab, or a biosimilar thereof. In some embodiments, the CDRs are defined by the Kabat numbering scheme.

In some embodiments, the anti-CD20 antibody comprises a heavy chain variable region or a light chain variable region of the anti-CD20 antibody rituximab, or a biosimilar thereof. In some embodiments, the anti-CD20 antibody comprises a heavy chain variable region and a light chain variable region of the anti-CD20 antibody rituximab, or a biosimilar thereof.

In some embodiments, the anti-CD20 antibody is rituximab, or a biosimilar thereof. In some embodiments, the anti-CD20 antibody is rituximab.

In some embodiments, the anti-CD20 antibody is a biosimilar of rituximab. Biosimilars of rituximab include TRUXIMA® (Celltrion), RUXIENCE® (Pfizer), and RIXATHON® (Sandoz). In some embodiments, the biosimilar of rituximab is selected from the group consisting of TRUXIMA®, RUXIENCE® and RIXATHON®. In some embodiments, the biosimilar of rituximab is TRUXIMA®. In some embodiments, the biosimilar of rituximab is RUXIENCE®. In some embodiments, the biosimilar of rituximab is RIXATHON®.

Anti-CD20 antibodies of the present invention may also be described or specified in terms of their binding affinity to CD20 (e.g., human CD20). Preferred binding affinities include those with a dissociation constant or K_{D} less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁵ M, 10⁻⁶ M, 5×10⁻⁶ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgAl and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The antibodies also include derivatives that are modified, i.e., by the covalent attachment of any type of molecule to the antibody such that covalent attachment does not prevent the antibody from binding to CD20. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The CD20-binding agent can optionally include an antibody effector domain that mediates or stimulates an ADCC, ADCP and/or CDC response against a CD20-expressing target cell. The effector domain(s) can be, for example, an Fc domain or domains of an Ig molecule. Such a CD20-binding agent can exert a cytotoxic or cytostatic effect on CD20-expressing cells,

The effector domains of the anti-CD20 antibody can be from any suitable human immunoglobulin isotype. For example, the ability of human immunoglobulin to mediate CDC and ADCC/ADCP is generally in the order of IgM≈IgG1≈IgG3>IgG2>IgG4 and IgG1≈IgG3>IgG2/IgM/IgG4, respectively. A CD20-binding polypeptide can be expressed as a recombinant fusion protein comprising of the appropriate constant domains to yield the desired effector function(s). Upon binding to target cells, the anti-CD20 antibodies or derivatives can trigger in vitro and in vivo target cell destruction through an antibody effector function, such as ADCC, CDC, and ADCP.

### D. Methods of Treatment

The invention provides methods for treating non-Hodgkin lymphoma, such as diffuse large B-cell lymphoma (CLBCL) in a subject with lenalidomide, or salt or solvate thereof, and an antibody-drug conjugate that binds to CD30 as described herein. In some embodiments, the antibody-drug conjugate that binds to CD30 comprises an anti-CD30 antibody or an antigen-binding fragment thereof conjugated to a monomethyl auristatin or a functional analog thereof or a functional derivative thereof. In some embodiments, the anti-CD30 antibody of the antibody-drug conjugate comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6. In some embodiments, the anti-CD30 antibody of the antibody-drug conjugate comprises a heavy chain variable region comprising an amino acid sequence at least 85% identical to the amino acid sequence of SEQ ID NO: 7 and a light chain variable region comprising an amino acid sequence at least 85% identical to the amino acid sequence of SEQ ID NO: 8. In some embodiments, the anti-CD30 antibody of the antibody-drug conjugate comprises a heavy chain variable region comprising the amino acid sequence of SEQ ID NO: 7 and a light chain variable region comprising the amino acid sequence of SEQ ID NO: 8. In some embodiments, the antibody-drug conjugate is brentuximab vedotin. In some embodiments, the non-Hodgkin lymphoma is diffuse large B-cell lymphoma (DLBCL). In some embodiments, the DLBCL is relapsed DLBCL. In some embodiments, the DLBCL is refractory DLBCL. In some embodiments, the DLBCL is germinal-center B-cell like (GCB). In some embodiments, the DLBCL is non-GCB. In some embodiments, the non-Hodgkin lymphoma is an advanced stage non-Hodgkin lymphoma. In some embodiments, the advanced stage non-Hodgkin lymphoma is a stage 3 or stage 4 non-Hodgkin lymphoma. In some embodiments, the advanced stage non-Hodgkin lymphoma is metastatic non-Hodgkin lymphoma. In some embodiments, the non-Hodgkin lymphoma is recurrent non-Hodgkin lymphoma. In some embodiments, the subject previously received allogenic stem cell transplant to treat the non-Hodgkin lymphoma. In some embodiments, the subject previously received autologous stem cell transplant to treat the non-Hodgkin lymphoma. In some embodiments, the subject relapsed following stem cell transplant. In some embodiments, the subject previously received CAR-T therapy. In some embodiments, the subject relapsed after CAR-T therapy. In some embodiments, the subject has not been previously treated with lenalidomide, or salt or solvate thereof. In some embodiments, the subject has not been previously treated with an antibody-drug conjugate that binds to CD30. In some embodiments, at least 1% of the non-Hodgkin lymphoma cells in the subject express CD30. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the non-Hodgkin lymphoma cells in the subject express CD30. In a particular embodiment, the subject is a human. In certain embodiments, the subject is further administered granulocyte colony-stimulating factor (G-CSF). In certain embodiments, the G-CSF is administered prophylactically. In certain embodiments, the G-CSF is administered 1 to 3 days after the administration of the anti-CD30 antibody-drug conjugate. In certain embodiments, the G-CSF is administered 1 day after the administration of the anti-CD30 antibody-drug conjugate. In certain embodiments, the G-CSF is administered 2 days after the administration of the anti-CD30 antibody-drug conjugate. In certain embodiments, the G-CSF is administered 3 days after the administration of the anti-CD30 antibody-drug conjugate. In certain embodiments, the G-CSF is recombinant human G-CSF. In certain embodiments, the GCSF is filgrastim (NEUPOGEN®). In certain embodiments, the G-CSF is PEG-filgrastim (NEULASTA®). In certain embodiments, the G-CSF is lenograstim (GRANOCYTE®). In certain embodiments, the G-CSF is tbo-filgrastim (GRANIX®).

In some embodiments, the invention provides methods for treating non-Hodgkin lymphoma, such as diffuse large B-cell lymphoma (CLBCL) in a subject with lenalidomide, or salt or solvate thereof, an antibody-drug conjugate that binds to CD30 as described herein, and an anti-CD20 antibody or antigen-binding fragment thereof as described herein. In some embodiments, the antibody-drug conjugate that binds to CD30 comprises an anti-CD30 antibody or an antigen-binding fragment thereof conjugated to a monomethyl auristatin or a functional analog thereof or a functional derivative thereof. In some embodiments, the anti-CD30 antibody of the antibody-drug conjugate comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6. In some embodiments, the anti-CD30 antibody of the antibody-drug conjugate comprises a heavy chain variable region comprising an amino acid sequence at least 85% identical to the amino acid sequence of SEQ ID NO: 7 and a light chain variable region comprising an amino acid sequence at least 85% identical to the amino acid sequence of SEQ ID NO: 8. In some embodiments, the anti-CD30 antibody of the antibody-drug conjugate comprises a heavy chain variable region comprising the amino acid sequence of SEQ ID NO: 7 and a light chain variable region comprising the amino acid sequence of SEQ ID NO: 8. In some embodiments, the antibody-drug conjugate is brentuximab vedotin. In some embodiments, the anti-CD20 antibody or antigen-binding fragment thereof comprises a heavy chain variable region comprising the three CDRs of SEQ ID NO:17, a light chain variable region comprising the three CDRs of SEQ ID NO:18, wherein the CDRs of the anti-CD20 antibody are defined by the Kabat numbering scheme. In some embodiments, the anti-CD20 antibody or antigen-binding fragment thereof comprises a heavy chain variable region comprising an amino acid sequence at least 85% identical to the amino acid sequence of SEQ ID NO:17 and a light chain variable region comprising an amino acid sequence at least 85% identical to the amino acid sequence of SEQ ID NO:18. In some embodiments, the anti-CD20 antibody or antigen-binding fragment thereof comprises a heavy chain variable region comprising the amino acid sequence of SEQ ID NO:17 and a light chain variable region comprising the amino acid sequence of SEQ ID NO:18. In some embodiments, the anti-CD20 antibody or antigen-binding fragment thereof is rituximab or a biosimilar thereof. In some embodiments, the anti-CD20 antibody or antigen-binding fragment thereof is rituximab. In some embodiments, the non-Hodgkin lymphoma is diffuse large B-cell lymphoma (DLBCL). In some embodiments, the DLBCL is relapsed DLBCL. In some embodiments, the DLBCL is refractory DLBCL. In some embodiments, the DLBCL is germinal-center B-cell like (GCB). In some embodiments, the DLBCL is non-GCB. In some embodiments, the non-Hodgkin lymphoma is an advanced stage non-Hodgkin lymphoma. In some embodiments, the advanced stage non-Hodgkin lymphoma is a stage 3 or stage 4 non-Hodgkin lymphoma. In some embodiments, the advanced stage non-Hodgkin lymphoma is metastatic non-Hodgkin lymphoma. In some embodiments, the non-Hodgkin lymphoma is recurrent non-Hodgkin lymphoma. In some embodiments, the subject previously received allogenic stem cell transplant to treat the non-Hodgkin lymphoma. In some embodiments, the subject previously received autologous stem cell transplant to treat the non-Hodgkin lymphoma. In some embodiments, the subject relapsed following stem cell transplant. In some embodiments, the subject previously received CAR-T therapy. In some embodiments, the subject relapsed after CAR-T therapy. In some embodiments, the subject has not been previously treated with lenalidomide, or salt or solvate thereof. In some embodiments, the subject has not been previously treated with an antibody-drug conjugate that binds to CD30. In some embodiments, at least 1% of the non-Hodgkin lymphoma cells in the subject express CD30. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the non-Hodgkin lymphoma cells in the subject express CD30. In a particular embodiment, the subject is a human. In certain embodiments, the subject is further administered granulocyte colony-stimulating factor (G-CSF). In certain embodiments, the G-CSF is administered prophylactically. In certain embodiments, the G-CSF is administered 1 to 3 days after the administration of the anti-CD30 antibody-drug conjugate. In certain embodiments, the G-CSF is administered 1 day after the administration of the anti-CD30 antibody-drug conjugate. In certain embodiments, the G-CSF is administered 2 days after the administration of the anti-CD30 antibody-drug conjugate. In certain embodiments, the G-CSF is administered 3 days after the administration of the anti-CD30 antibody-drug conjugate. In certain embodiments, the G-CSF is recombinant human G-CSF. In certain embodiments, the GCSF is filgrastim (NEUPOGEN®). In certain embodiments, the G-CSF is PEG-filgrastim (NEULASTA®). In certain embodiments, the G-CSF is lenograstim (GRANOCYTE®). In certain embodiments, the G-CSF is tbo-filgrastim (GRANIX®).

### E. Routes of Administration

Lenalidomide, or salt or solvate thereof, an antibody-drug conjugate that binds to CD30 as described herein, and an anti-CD20 antibody or antigen-binding fragment thereof as described herein can be administered by any suitable route and mode. Suitable routes of administering lenalidomide, or salt or solvate thereof, antibodies and/or antibody-drug conjugate of the present invention are well known in the art and may be selected by those of ordinary skill in the art. In one embodiment, lenalidomide, or salt or solvate thereof, antibodies described herein and/or antibody-drug conjugate described herein are administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, lenalidomide, or salt or solvate thereof, is administered orally. In some embodiments, the route of administration of an anti-CD30 antibody-drug conjugate or antigen-binding fragment described herein is intravenous infusion. In some embodiments, the route of administration of an anti-CD20 antibody or antigen-binding fragment described herein is intravenous injection or infusion. In some embodiments, the route of administration of an anti-CD20 antibody or antigen-binding fragment described herein is intravenous infusion. In some embodiments, the route of administration of an anti-CD20 antibody or antigen-binding fragment described herein is subcutaneous injection.

### F. Dosage and Frequency of Administration

In one aspect, the present invention provides for methods of treating a subject with non-Hodgkin lymphoma, such as DLBCL, as described herein with a particular dose of lenalidomide, or salt or solvate thereof, and an anti-CD30 antibody-drug conjugate or antigen-binding fragment thereof as described herein, wherein the subject is administered the lenalidomide, or salt or solvate thereof, and the anti-CD30 antibody-drug conjugate or antigen-binding fragment thereof as described herein with particular frequencies. In some embodiments, the method further comprises the administration of an anti-CD20 antibody or antigen-binding fragment thereof as described herein with a particular frequency.

In one embodiment of the methods or uses or product for uses provided herein, lenalidomide, or salt or solvate thereof, is administered to the subject in a dose ranging from about 1.0 mg to about 40 mg. In certain embodiments, the dose is about 1.0 mg, about 2.0 mg, about 3.0 mg, about 4.0 mg, about 5.0 mg, about 6.0 mg, about 7.0 mg, about 8.0 mg, about 9.0 mg, about 10.0 mg, about 11.0 mg, about 12.0 mg, about 13.0 mg, about 14.0 mg, about 15.0 mg, about 16.0 mg, about 17.0 mg, about 18.0 mg, about 19.0 mg, about 20.0 mg, about 21.0 mg, about 22.0 mg, about 23.0 mg, about 24.0 mg, about 25.0 mg, about 26.0 mg, about 27.0 mg, about 28.0 mg, about 29.0 mg, about 30.0 mg, about 31.0 mg, about 32.0 mg, about 33.0 mg, about 34.0 mg, about 35.0 mg, about 36.0 mg, about 37.0 mg, about 38.0 mg, about 39.0 mg, or about 40.0 mg. In one embodiment, the dose is about 5 mg. In one embodiment, the dose is about 10 mg. In one embodiment, the dose is about 15 mg. In one embodiment, the dose is about 20 mg. In one embodiment, the dose is about 25 mg. In one embodiment of the methods or uses or product for uses provided herein, lenalidomide, or salt or solvate thereof, is administered to the subject in a dose ranging from 1.0 mg to 40 mg. In certain embodiments, the dose is 1.0 mg, 2.0 mg, 3.0 mg, 4.0 mg, 5.0 mg, 6.0 mg, 7.0 mg, 8.0 mg, 9.0 mg, 10.0 mg, 11.0 mg, 12.0 mg, 13.0 mg, 14.0 mg, 15.0 mg, 16.0 mg, 17.0 mg, 18.0 mg, 19.0 mg, 20.0 mg, 21.0 mg, 22.0 mg, 23.0 mg, 24.0 mg, 25.0 mg, 26.0 mg, 27.0 mg, 28.0 mg, 29.0 mg, 30.0 mg, 31.0 mg, 32.0 mg, 33.0 mg, 34.0 mg, 35.0 mg, 36.0 mg, 37.0 mg, 38.0 mg, 39.0 mg, or 40.0 mg. In one embodiment, the dose is 5 mg. In one embodiment, the dose is 10 mg. In one embodiment, the dose is 15 mg. In one embodiment, the dose is 20 mg. In one embodiment, the dose is 25 mg.

In one embodiment of the methods or uses or product for uses provided herein, lenalidomide, or salt or solvate thereof, is administered to the subject once about every 1 to 3 days. In certain embodiments, lenalidomide, or salt or solvate thereof, is administered to the subject once about every day. In certain embodiments, lenalidomide, or salt or solvate thereof, is administered to the subject once about every 2 days. In certain embodiments, lenalidomide, or salt or solvate thereof, is administered to the subject once about every 3 days. In one embodiment of the methods or uses or product for uses provided herein, lenalidomide, or salt or solvate thereof, is administered to the subject once every 1 to 3 days. In certain embodiments, lenalidomide, or salt or solvate thereof, is administered to the subject once every day. In certain embodiments, lenalidomide, or salt or solvate thereof, is administered to the subject once every 2 days. In certain embodiments, lenalidomide, or salt or solvate thereof, is administered to the subject once every 3 days. In some embodiments, lenalidomide, or salt or solvate thereof, is administered to the subject at a dose of about 5 mg once about every day. In some embodiments, lenalidomide, or salt or solvate thereof, is administered to the subject at a dose of about 10 mg once about every day. In some embodiments, lenalidomide, or salt or solvate thereof, is administered to the subject at a dose of about 15 mg once about every day. In some embodiments, lenalidomide, or salt or solvate thereof, is administered to the subject at a dose of about 20 mg once about every day. In some embodiments, lenalidomide, or salt or solvate thereof, is administered to the subject at a dose of about 25 mg once about every day. In some embodiments, lenalidomide, or salt or solvate thereof, is administered to the subject at a dose of 5 mg once every day. In some embodiments, lenalidomide, or salt or solvate thereof, is administered to the subject at a dose of 10 mg once every day. In some embodiments, lenalidomide, or salt or solvate thereof, is administered to the subject at a dose of 15 mg once every day. In some embodiments, lenalidomide, or salt or solvate thereof, is administered to the subject at a dose of 20 mg once every day. In some embodiments, lenalidomide, or salt or solvate thereof, is administered to the subject at a dose of 25 mg once about every day.

In one embodiment of the methods or uses or product for uses provided herein, an anti-CD30 antibody-drug conjugate or antigen-binding fragment thereof as described herein is administered to the subject at a dose ranging from about 0.6 mg/kg to about 2.3 mg/kg of the subject's body weight. In certain embodiments, the dose is about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1.0 mg/kg, about 1.05 mg/kg, about 1.1 mg/kg, about 1.15 mg/kg, about 1.2 mg/kg, about 1.25 mg/kg, about 1.3 mg/kg, about 1.35 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, or about 2.3 mg/kg. In one embodiment, the dose is about 0.65 mg/kg. In one embodiment, the dose is about 0.9 mg/kg. In one embodiment, the dose is about 1.2 mg/kg. In certain embodiments, the dose is 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.05 mg/kg, 1.1 mg/kg, 1.15 mg/kg, 1.2 mg/kg, 1.25 mg/kg, 1.3 mg/kg, 1.35 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, or 2.3 mg/kg. In one embodiment, the dose is 0.65 mg/kg. In one embodiment, the dose is 0.9 mg/kg. In one embodiment, the dose is 1.2 mg/kg. In some embodiments, the dose is 0.9 mg/kg and the anti-CD30 antibody-drug conjugate is brentuximab vedotin. In some embodiments, the dose is 1.2 mg/kg and the anti-CD30 antibody-drug conjugate is brentuximab vedotin. In some embodiments, for a subject weighing more than 100 kg, the dose of the anti-CD30 antibody-drug conjugate administered is the amount that would be administered if the subject weighed 100 kg. In some embodiments, for a subject weighing more than 100 kg, the dose of the anti-CD30 antibody-drug conjugate administered is 90 mg. In some embodiments, for a subject weighing more than 100 kg, the dose of the anti-CD30 antibody-drug conjugate administered is 120 mg.

In one embodiment of the methods or uses or product for uses provided herein, an anti-CD30 antibody-drug conjugate or antigen-binding fragment thereof as described herein is administered to the subject once about every 1 to 4 weeks. In certain embodiments, an anti-CD30 antibody-drug conjugate or antigen-binding fragment thereof as described herein is administered once about every 1 week, once about every 2 weeks, once about every 3 weeks or once about every 4 weeks. In one embodiment, an anti-CD30 antibody-drug conjugate or antigen-binding fragment thereof as described herein is administered once about every 3 weeks. In one embodiment, an anti-CD30 antibody-drug conjugate or antigen-binding fragment thereof as described herein is administered once every 3 weeks. In some embodiments, the dose is about 0.65 mg/kg and is administered once about every 1 week. In some embodiments, the dose is about 0.65 mg/kg and is administered once about every 2 weeks. In some embodiments, the dose is about 0.65 mg/kg and is administered once about every 3 weeks. In some embodiments, the dose is about 0.65 mg/kg and is administered once about every 4 weeks. In some embodiments, the dose is about 0.7 mg/kg and is administered once about every 1 week. In some embodiments, the dose is about 0.7 mg/kg and is administered once about every 2 weeks. In some embodiments, the dose is about 0.7 mg/kg and is administered once about every 3 weeks. In some embodiments, the dose is about 0.7 mg/kg and is administered once about every 4 weeks. In some embodiments, the dose is about 0.75 mg/kg and is administered once about every 1 week. In some embodiments, the dose is about 0.75 mg/kg and is administered once about every 2 weeks. In some embodiments, the dose is about 0.75 mg/kg and is administered once about every 3 weeks. In some embodiments, the dose is about 0.75 mg/kg and is administered once about every 4 weeks. In some embodiments, the dose is about 0.8 mg/kg and is administered once about every 1 week. In some embodiments, the dose is about 0.8 mg/kg and is administered once about every 2 weeks. In some embodiments, the dose is about 0.8 mg/kg and is administered once about every 3 weeks. In some embodiments, the dose is about 0.8 mg/kg and is administered once about every 4 weeks. In some embodiments, the dose is about 0.85 mg/kg and is administered once about every 1 week. In some embodiments, the dose is about 0.85 mg/kg and is administered once about every 2 weeks. In some embodiments, the dose is about 0.85 mg/kg and is administered once about every 3 weeks. In some embodiments, the dose is about 0.85 mg/kg and is administered once about every 4 weeks. In some embodiments, the dose is about 0.9 mg/kg and is administered once about every 1 week. In some embodiments, the dose is about 0.9 mg/kg and is administered once about every 2 weeks. In some embodiments, the dose is about 0.9 mg/kg and is administered once about every 3 weeks. In some embodiments, the dose is about 0.9 mg/kg and is administered once about every 4 weeks. In some embodiments, the dose is about 0.95 mg/kg and is administered once about every 1 week. In some embodiments, the dose is about 0.95 mg/kg and is administered once about every 2 weeks. In some embodiments, the dose is about 0.95 mg/kg and is administered once about every 3 weeks. In some embodiments, the dose is about 0.95 mg/kg and is administered once about every 4 weeks. In some embodiments, the dose is about 1.0 mg/kg and is administered once about every 1 week. In some embodiments, the dose is about 1.0 mg/kg and is administered once about every 2 weeks. In some embodiments, the dose is about 1.0 mg/kg and is administered once about every 3 weeks. In some embodiments, the dose is about 1.0 mg/kg and is administered once about every 4 weeks. In some embodiments, the dose is about 1.05 mg/kg and is administered once about every 1 week. In some embodiments, the dose is about 1.05 mg/kg and is administered once about every 2 weeks. In some embodiments, the dose is about 1.05 mg/kg and is administered once about every 3 weeks. In some embodiments, the dose is about 1.05 mg/kg and is administered once about every 4 weeks. In some embodiments, the dose is about 1.1 mg/kg and is administered once about every 1 week. In some embodiments, the dose is about 1.1 mg/kg and is administered once about every 2 weeks. In some embodiments, the dose is about 1.1 mg/kg and is administered once about every 3 weeks. In some embodiments, the dose is about 1.1 mg/kg and is administered once about every 4 weeks. In some embodiments, the dose is about 1.15 mg/kg and is administered once about every 1 week. In some embodiments, the dose is about 1.15 mg/kg and is administered once about every 2 weeks. In some embodiments, the dose is about 1.15 mg/kg and is administered once about every 3 weeks. In some embodiments, the dose is about 1.15 mg/kg and is administered once about every 4 weeks. In some embodiments, the dose is about 1.2 mg/kg and is administered once about every 1 week. In some embodiments, the dose is about 1.2 mg/kg and is administered once about every 2 weeks. In some embodiments, the dose is about 1.2 mg/kg and is administered once about every 3 weeks. In some embodiments, the dose is about 1.2 mg/kg and is administered once about every 4 weeks. In some embodiments, the dose is about 1.25 mg/kg and is administered once about every 1 week. In some embodiments, the dose is about 1.25 mg/kg and is administered once about every 2 weeks. In some embodiments, the dose is about 1.25 mg/kg and is administered once about every 3 weeks. In some embodiments, the dose is about 1.25 mg/kg and is administered once about every 4 weeks. In some embodiments, the dose is about 1.3 mg/kg and is administered once about every 1 week. In some embodiments, the dose is about 1.3 mg/kg and is administered once about every 2 weeks. In some embodiments, the dose is about 1.3 mg/kg and is administered once about every 3 weeks. In some embodiments, the dose is about 1.3 mg/kg and is administered once about every 4 weeks. In some embodiments, the dose is about 1.35 mg/kg and is administered once about every 1 week. In some embodiments, the dose is about 1.35 mg/kg and is administered once about every 2 weeks. In some embodiments, the dose is about 1.35 mg/kg and is administered once about every 3 weeks. In some embodiments, the dose is about 1.35 mg/kg and is administered once about every 4 weeks. In some embodiments, the dose is about 1.4 mg/kg and is administered once about every 1 week. In some embodiments, the dose is about 1.4 mg/kg and is administered once about every 2 weeks. In some embodiments, the dose is about 1.4 mg/kg and is administered once about every 3 weeks. In some embodiments, the dose is about 1.4 mg/kg and is administered once about every 4 weeks. In some embodiments, the dose is about 1.5 mg/kg and is administered once about every 1 week. In some embodiments, the dose is about 1.5 mg/kg and is administered once about every 2 weeks. In some embodiments, the dose is about 1.5 mg/kg and is administered once about every 3 weeks. In some embodiments, the dose is about 1.5 mg/kg and is administered once about every 4 weeks. In some embodiments, the dose is about 1.6 mg/kg and is administered once about every 1 week. In some embodiments, the dose is about 1.6 mg/kg and is administered once about every 2 weeks. In some embodiments, the dose is about 1.6 mg/kg and is administered once about every 3 weeks. In some embodiments, the dose is about 1.6 mg/kg and is administered once about every 4 weeks. In some embodiments, the dose is about 1.7 mg/kg and is administered once about every 1 week. In some embodiments, the dose is about 1.7 mg/kg and is administered once about every 2 weeks. In some embodiments, the dose is about 1.7 mg/kg and is administered once about every 3 weeks. In some embodiments, the dose is about 1.7 mg/kg and is administered once about every 4 weeks. In some embodiments, the dose is about 1.8 mg/kg and is administered once about every 1 week. In some embodiments, the dose is about 1.8 mg/kg and is administered once about every 2 weeks. In some embodiments, the dose is about 1.8 mg/kg and is administered once about every 3 weeks. In some embodiments, the dose is about 1.8 mg/kg and is administered once about every 4 weeks. In some embodiments, the dose is about 1.9 mg/kg and is administered once about every 1 week. In some embodiments, the dose is about 1.9 mg/kg and is administered once about every 2 weeks. In some embodiments, the dose is about 1.9 mg/kg and is administered once about every 3 weeks. In some embodiments, the dose is about 1.9 mg/kg and is administered once about every 4 weeks. In some embodiments, the dose is about 2.0 mg/kg and is administered once about every 1 week. In some embodiments, the dose is about 2.0 mg/kg and is administered once about every 2 weeks. In some embodiments, the dose is about 2.0 mg/kg and is administered once about every 3 weeks. In some embodiments, the dose is about 2.0 mg/kg and is administered once about every 4 weeks. In some embodiments, the dose is about 2.1 mg/kg and is administered once about every 1 week.

In some embodiments, the dose is about 2.1 mg/kg and is administered once about every 2 weeks. In some embodiments, the dose is about 2.1 mg/kg and is administered once about every 3 weeks. In some embodiments, the dose is about 2.1 mg/kg and is administered once about every 4 weeks. In some embodiments, the dose is about 2.2 mg/kg and is administered once about every 1 week. In some embodiments, the dose is about 2.2 mg/kg and is administered once about every 2 weeks. In some embodiments, the dose is about 2.2 mg/kg and is administered once about every 3 weeks. In some embodiments, the dose is about 2.2 mg/kg and is administered once about every 4 weeks. In some embodiments, the dose is about 2.3 mg/kg and is administered once about every 1 week. In some embodiments, the dose is about 2.3 mg/kg and is administered once about every 2 weeks. In some embodiments, the dose is about 2.3 mg/kg and is administered once about every 3 weeks. In some embodiments, the dose is about 2.3 mg/kg and is administered once about every 4 weeks. In some embodiments, the dose is 0.65 mg/kg and is administered once about every 1 week. In some embodiments, the dose is 0.65 mg/kg and is administered once about every 2 weeks. In some embodiments, the dose is 0.65 mg/kg and is administered once about every 3 weeks. In some embodiments, the dose is 0.65 mg/kg and is administered once about every 4 weeks. In some embodiments, the dose is 0.7 mg/kg and is administered once about every 1 week. In some embodiments, the dose is 0.7 mg/kg and is administered once about every 2 weeks. In some embodiments, the dose is 0.7 mg/kg and is administered once about every 3 weeks. In some embodiments, the dose is 0.7 mg/kg and is administered once about every 4 weeks. In some embodiments, the dose is 0.75 mg/kg and is administered once about every 1 week. In some embodiments, the dose is 0.75 mg/kg and is administered once about every 2 weeks. In some embodiments, the dose is 0.75 mg/kg and is administered once about every 3 weeks. In some embodiments, the dose is 0.75 mg/kg and is administered once about every 4 weeks. In some embodiments, the dose is 0.8 mg/kg and is administered once about every 1 week. In some embodiments, the dose is 0.8 mg/kg and is administered once about every 2 weeks. In some embodiments, the dose is 0.8 mg/kg and is administered once about every 3 weeks. In some embodiments, the dose is 0.8 mg/kg and is administered once about every 4 weeks. In some embodiments, the dose is 0.85 mg/kg and is administered once about every 1 week. In some embodiments, the dose is 0.85 mg/kg and is administered once about every 2 weeks. In some embodiments, the dose is 0.85 mg/kg and is administered once about every 3 weeks. In some embodiments, the dose is 0.85 mg/kg and is administered once about every 4 weeks. In some embodiments, the dose is 0.9 mg/kg and is administered once about every 1 week. In some embodiments, the dose is 0.9 mg/kg and is administered once about every 2 weeks. In some embodiments, the dose is 0.9 mg/kg and is administered once about every 3 weeks. In some embodiments, the dose is 0.9 mg/kg and is administered once about every 4 weeks. In some embodiments, the dose is 0.95 mg/kg and is administered once about every 1 week. In some embodiments, the dose is 0.95 mg/kg and is administered once about every 2 weeks. In some embodiments, the dose is 0.95 mg/kg and is administered once about every 3 weeks. In some embodiments, the dose is 0.95 mg/kg and is administered once about every 4 weeks. In some embodiments, the dose is 1.0 mg/kg and is administered once about every 1 week. In some embodiments, the dose is 1.0 mg/kg and is administered once about every 2 weeks. In some embodiments, the dose is 1.0 mg/kg and is administered once about every 3 weeks. In some embodiments, the dose is 1.0 mg/kg and is administered once about every 4 weeks. In some embodiments, the dose is 1.05 mg/kg and is administered once about every 1 week. In some embodiments, the dose is 1.05 mg/kg and is administered once about every 2 weeks. In some embodiments, the dose is 1.05 mg/kg and is administered once about every 3 weeks. In some embodiments, the dose is 1.05 mg/kg and is administered once about every 4 weeks. In some embodiments, the dose is 1.1 mg/kg and is administered once about every 1 week. In some embodiments, the dose is 1.1 mg/kg and is administered once about every 2 weeks. In some embodiments, the dose is 1.1 mg/kg and is administered once about every 3 weeks. In some embodiments, the dose is 1.1 mg/kg and is administered once about every 4 weeks. In some embodiments, the dose is 1.15 mg/kg and is administered once about every 1 week. In some embodiments, the dose is 1.15 mg/kg and is administered once about every 2 weeks. In some embodiments, the dose is 1.15 mg/kg and is administered once about every 3 weeks. In some embodiments, the dose is 1.15 mg/kg and is administered once about every 4 weeks. In some embodiments, the dose is 1.2 mg/kg and is administered once about every 1 week. In some embodiments, the dose is 1.2 mg/kg and is administered once about every 2 weeks. In some embodiments, the dose is 1.2 mg/kg and is administered once about every 3 weeks. In some embodiments, the dose is 1.2 mg/kg and is administered once about every 4 weeks. In some embodiments, the dose is 1.25 mg/kg and is administered once about every 1 week. In some embodiments, the dose is 1.25 mg/kg and is administered once about every 2 weeks. In some embodiments, the dose is 1.25 mg/kg and is administered once about every 3 weeks. In some embodiments, the dose is 1.25 mg/kg and is administered once about every 4 weeks. In some embodiments, the dose is 1.3 mg/kg and is administered once about every 1 week. In some embodiments, the dose is 1.3 mg/kg and is administered once about every 2 weeks. In some embodiments, the dose is 1.3 mg/kg and is administered once about every 3 weeks. In some embodiments, the dose is 1.3 mg/kg and is administered once about every 4 weeks. In some embodiments, the dose is 1.4 mg/kg and is administered once about every 1 week. In some embodiments, the dose is 1.4 mg/kg and is administered once about every 2 weeks. In some embodiments, the dose is 1.4 mg/kg and is administered once about every 3 weeks. In some embodiments, the dose is 1.4 mg/kg and is administered once about every 4 weeks. In some embodiments, the dose is 1.5 mg/kg and is administered once about every 1 week. In some embodiments, the dose is 1.5 mg/kg and is administered once about every 2 weeks. In some embodiments, the dose is 1.5 mg/kg and is administered once about every 3 weeks. In some embodiments, the dose is 1.5 mg/kg and is administered once about every 4 weeks. In some embodiments, the dose is 1.6 mg/kg and is administered once about every 1 week. In some embodiments, the dose is 1.6 mg/kg and is administered once about every 2 weeks. In some embodiments, the dose is 1.6 mg/kg and is administered once about every 3 weeks. In some embodiments, the dose is 1.6 mg/kg and is administered once about every 4 weeks. In some embodiments, the dose is 1.7 mg/kg and is administered once about every 1 week. In some embodiments, the dose is 1.7 mg/kg and is administered once about every 2 weeks. In some embodiments, the dose is 1.7 mg/kg and is administered once about every 3 weeks. In some embodiments, the dose is 1.7 mg/kg and is administered once about every 4 weeks. In some embodiments, the dose is 1.8 mg/kg and is administered once about every 1 week. In some embodiments, the dose is 1.8 mg/kg and is administered once about every 2 weeks. In some embodiments, the dose is 1.8 mg/kg and is administered once about every 3 weeks. In some embodiments, the dose is 1.8 mg/kg and is administered once about every 4 weeks. In some embodiments, the dose is 1.9 mg/kg and is administered once about every 1 week. In some embodiments, the dose is 1.9 mg/kg and is administered once about every 2 weeks. In some embodiments, the dose is 1.9 mg/kg and is administered once about every 3 weeks. In some embodiments, the dose is 1.9 mg/kg and is administered once about every 4 weeks. In some embodiments, the dose is 2.0 mg/kg and is administered once about every 1 week. In some embodiments, the dose is 2.0 mg/kg and is administered once about every 2 weeks. In some embodiments, the dose is 2.0 mg/kg and is administered once about every 3 weeks. In some embodiments, the dose is 2.0 mg/kg and is administered once about every 4 weeks. In some embodiments, the dose is 2.1 mg/kg and is administered once about every 1 week. In some embodiments, the dose is 2.1 mg/kg and is administered once about every 2 weeks. In some embodiments, the dose is 2.1 mg/kg and is administered once about every 3 weeks. In some embodiments, the dose is 2.1 mg/kg and is administered once about every 4 weeks. In some embodiments, the dose is 2.2 mg/kg and is administered once about every 1 week. In some embodiments, the dose is 2.2 mg/kg and is administered once about every 2 weeks. In some embodiments, the dose is 2.2 mg/kg and is administered once about every 3 weeks. In some embodiments, the dose is 2.2 mg/kg and is administered once about every 4 weeks. In some embodiments, the dose is 2.3 mg/kg and is administered once about every 1 week. In some embodiments, the dose is 2.3 mg/kg and is administered once about every 2 weeks. In some embodiments, the dose is 2.3 mg/kg and is administered once about every 3 weeks. In some embodiments, the dose is 2.3 mg/kg and is administered once about every 4 weeks. In some embodiments, the dose is 1.2 mg/kg and is administered once about every 3 weeks (e.g., ±3 days). In some embodiments, the dose is 1.2 mg/kg and is administered once every 3 weeks. In some embodiments, the dose is 1.2 mg/kg and is administered once every 3 weeks and the antibody-drug conjugate is brentuximab vedotin. In some embodiments, the dose is 1.2 mg/kg and is administered once every 3 weeks and the antibody-drug conjugate is brentuximab vedotin and the dose is decreased to 0.9 mg/kg if one or more adverse events occur. In some embodiments, the dose is 1.2 mg/kg and is administered on about day 1 of about a 21-day treatment cycle and the antibody-drug conjugate is brentuximab vedotin. In some embodiments, the dose is 0.9 mg/kg and is administered on day 1 of a 21-day treatment cycle and the antibody-drug conjugate is brentuximab vedotin. In some embodiments, the dose is 1.2 mg/kg and is administered on about day 1 of about a 21-day treatment cycle and the antibody drug conjugate is brentuximab vedotin and the dose is decreased to 0.9 mg/kg if one or more adverse events occur. The present invention encompasses embodiments wherein the subject remains on the 21-day treatment cycle for at least 2, 3, 4, 5, 6, 7, 8, 9, 10, 11, 12 or more cycles. In another embodiment, the subject remains on the 21-day treatment cycle for between 2 and 48 cycles, such as between 2 and 36 cycles, such as between 2 and 24 cycles, such as between 2 and 15 cycles, such as between 2 and 12 cycles, such as 2 cycles, 3 cycles, 4 cycles, 5 cycles, 6 cycles, 7 cycles, 8 cycles, 9 cycles, 10 cycles, 11 cycles or 12 cycles. In some embodiments, the subject remains on the 21-day treatment cycle for 12 cycles or more, such as 16 cycles or more, such as 24 cycles or more, such as 36 cycles or more. In some embodiments, the 21-day treatment cycle is administered for no more than 3, no more than 4, no more than 5, or no more than 6 four-week treatment cycles. The number of treatment cycles suitable for any specific subject or group of subjects may be determined by a person of skill in the art, typically a physician. In some embodiments, for a subject weighing more than 100 kg, the dose of the anti-CD30 antibody-drug conjugate administered is the amount that would be administered if the subject weighed 100 kg. In some embodiments, for a subject weighing more than 100 kg, the dose of the anti-CD30 antibody-drug conjugate administered is 90 mg. In some embodiments, for a subject weighing more than 100 kg, the dose of the anti-CD30 antibody-drug conjugate administered is 120 mg.

In some embodiments, the dose of the anti-CD30 antibody-drug conjugate described herein is 1.2 mg/kg and is administered once about every 3 weeks (e.g., ±3 days) and the dose of lenalidomide, or salt or solvate thereof, is 20 mg and is administered once about every day. In some embodiments, the dose of the anti-CD30 antibody-drug conjugate described herein is 1.2 mg/kg and is administered once every 3 weeks and the dose of lenalidomide, or salt or solvate thereof, is 20 mg and is administered once every day. In some embodiments, the dose of the anti-CD30 antibody-drug conjugate is 1.2 mg/kg and is administered once every 3 weeks and the antibody-drug conjugate is brentuximab vedotin and the dose of lenalidomide, or salt or solvate thereof, is 20 mg and is administered once every day.

In some embodiments, the dose of the anti-CD30 antibody-drug conjugate described herein is 0.9 mg/kg and is administered once about every 3 weeks (e.g., ±3 days) and the dose of lenalidomide, or salt or solvate thereof, is 20 mg and is administered once about every day. In some embodiments, the dose of the anti-CD30 antibody-drug conjugate described herein is 0.9 mg/kg and is administered once every 3 weeks and the dose of lenalidomide, or salt or solvate thereof, is 20 mg and is administered once every day. In some embodiments, the dose of the anti-CD30 antibody-drug conjugate is 0.9 mg/kg and is administered once every 3 weeks and the antibody-drug conjugate is brentuximab vedotin and the dose of lenalidomide, or salt or solvate thereof, is 20 mg and is administered once every day.

In some embodiments, the method further comprises the administration of an anti-CD20 antibody or antigen-binding fragment thereof as described herein with a particular frequency. In certain embodiments, an anti-CD20 antibody or antigen-binding fragment thereof as described herein is administered to the subject based on the subject's body surface area. In one embodiment of the methods or uses or product for uses provided herein, an anti-CD20 antibody or antigen-binding fragment thereof as described herein is administered to the subject in a dose ranging from about 100 mg/m² to about 500 mg/m² of the subject's body surface area. In certain embodiments, the dose is about 100 mg/m², about 150 mg/m², about 200 mg/m², about 250 mg/m², about 275 mg/m², about 300 mg/m², about 325 mg/m², about 350 mg/m², about 375 mg/m², about 400 mg/m², about 425 mg/m², about 450 mg/m², about 475 mg/m², or about 500 mg/m². In one embodiment, an anti-CD20 antibody or antigen-binding fragment thereof as described herein is administered to the subject in a dose of about 375 mg/m² of the subject's body surface area. In some embodiments, the anti-CD20 antibody or antigen-binding fragment thereof as described herein is administered to the subject in a dose of about 375 mg/m² of the subject's body surface area by intravenous infusion. In some embodiments, the anti-CD20 antibody or antigen-binding fragment thereof as described herein is administered to the subject in a dose of about 375 mg/m² of the subject's body surface area by intravenous infusion and the anti-CD20 antibody or antigen-binding fragment thereof is rituximab.

In some embodiments, the method further comprises the administration of an anti-CD20 antibody or antigen-binding fragment thereof as described herein with a particular frequency. In certain embodiments, an anti-CD20 antibody or antigen-binding fragment thereof as described herein is administered to the subject based on the subject's body surface area. In one embodiment of the methods or uses or product for uses provided herein, an anti-CD20 antibody or antigen-binding fragment thereof as described herein is administered to the subject in a dose ranging from 100 mg/m² to 500 mg/m² of the subject's body surface area. In certain embodiments, the dose is 100 mg/m², 150 mg/m², 200 mg/m², 250 mg/m², 275 mg/m², 300 mg/m², 325 mg/m², 350 mg/m², 375 mg/m², 400 mg/m², 425 mg/m², 450 mg/m², 475 mg/m², or 500 mg/m². In one embodiment, an anti-CD20 antibody or antigen-binding fragment thereof as described herein is administered to the subject in a dose of 375 mg/m² of the subject's body surface area. In some embodiments, the anti-CD20 antibody or antigen-binding fragment thereof as described herein is administered to the subject in a dose of 375 mg/m² of the subject's body surface area by intravenous infusion. In some embodiments, the anti-CD20 antibody or antigen-binding fragment thereof as described herein is administered to the subject in a dose of 375 mg/m² of the subject's body surface area by intravenous infusion and the anti-CD20 antibody or antigen-binding fragment thereof is rituximab.

In some embodiments, the method further comprises the administration of an anti-CD20 antibody or antigen-binding fragment thereof as described herein with a particular frequency. In certain embodiments, an anti-CD20 antibody or antigen-binding fragment thereof as described herein is administered to the subject at a dose ranging from about 500 mg to about 2000 mg. In certain embodiments, the dose is about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg. In some embodiments, the dose is about 1400 mg. In some embodiments, the dose is about 1400 mg and the anti-CD20 antibody or antigen-binding fragment thereof is rituximab. In some embodiments, the dose is about 1400 mg and is administered to the subject by subcutaneous injection. In some embodiments, the dose is about 1400 mg and is administered to the subject by subcutaneous injection and the anti-CD20 antibody or antigen-binding fragment thereof is rituximab.

In some embodiments, the method further comprises the administration of an anti-CD20 antibody or antigen-binding fragment thereof as described herein with a particular frequency. In certain embodiments, an anti-CD20 antibody or antigen-binding fragment thereof as described herein is administered to the subject at a dose ranging from 500 mg to 2000 mg. In certain embodiments, the dose is 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg. In some embodiments, the dose is 1400 mg. In some embodiments, the dose is 1400 mg and the anti-CD20 antibody or antigen-binding fragment thereof is rituximab. In some embodiments, the dose is 1400 mg and is administered to the subject by subcutaneous injection. In some embodiments, the dose is 1400 mg and is administered to the subject by subcutaneous injection and the anti-CD20 antibody or antigen-binding fragment thereof is rituximab.

In one embodiment of the methods or uses or product for uses provided herein, an anti-CD20 antibody or antigen-binding fragment thereof as described herein is administered to the subject once about every 1 to 4 weeks. In certain embodiments, an anti-CD20 antibody or antigen-binding fragment thereof as described herein is administered once about every 1 week, once about every 2 weeks, once about every 3 weeks or once about every 4 weeks. In one embodiment, an anti-CD20 antibody or antigen-binding fragment thereof as described herein is administered once about every 3 weeks. In one embodiment, an anti-CD20 antibody or antigen-binding fragment thereof as described herein is administered once every 3 weeks. In some embodiments, the dose is about 375 mg/m² of the subject's body surface area and is administered once about every 1 week. In some embodiments, the dose is about 375 mg/m² and is administered once about every 2 weeks. In some embodiments, the dose is about 375 mg/m² and is administered once about every 3 weeks. In some embodiments, the dose is about 375 mg/m² and is administered once about every 4 weeks.

In one embodiment of the methods or uses or product for uses provided herein, an anti-CD20 antibody or antigen-binding fragment thereof as described herein is administered to the subject once every 1 to 4 weeks. In certain embodiments, an anti-CD20 antibody or antigen-binding fragment thereof as described herein is administered once every 1 week, once every 2 weeks, once every 3 weeks or once every 4 weeks. In one embodiment, an anti-CD20 antibody or antigen-binding fragment thereof as described herein is administered once every 3 weeks. In one embodiment, an anti-CD20 antibody or antigen-binding fragment thereof as described herein is administered once every 3 weeks. In some embodiments, the dose is 375 mg/m² of the subject's body surface area and is administered once every 1 week. In some embodiments, the dose is about 375 mg/m² and is administered once every 2 weeks. In some embodiments, the dose is about 375 mg/m² and is administered once every 3 weeks. In some embodiments, the dose is about 375 mg/m² and is administered once every 4 weeks. In some embodiments, the dose is 375 mg/m² and is administered once about every 3 weeks (e.g., ±3 days). In some embodiments, the dose is 375 mg/m² and is administered once every 3 weeks. In some embodiments, the dose is 375 mg/m² and is administered once every 3 weeks and the anti-CD20 antibody or antigen-binding fragment thereof is rituximab. In some embodiments, the dose is 375 mg/m² and is administered on about day 1 of about a 21-day treatment cycle and the anti-CD20 antibody or antigen-binding fragment thereof is rituximab. In some embodiments, the dose is 375 mg/m² and is administered on day 1 of a 21-day treatment cycle and the anti-CD20 antibody or antigen-binding fragment thereof is rituximab. The present invention encompasses embodiments wherein the subject remains on the 21-day treatment cycle for at least 2, 3, 4, 5, 6, 7, 8, 9, 10, 11, 12 or more cycles. In another embodiment, the subject remains on the 21-day treatment cycle for between 2 and 48 cycles, such as between 2 and 36 cycles, such as between 2 and 24 cycles, such as between 2 and 15 cycles, such as between 2 and 12 cycles, such as 2 cycles, 3 cycles, 4 cycles, 5 cycles, 6 cycles, 7 cycles, 8 cycles, 9 cycles, 10 cycles, 11 cycles or 12 cycles. In some embodiments, the subject remains on the 21-day treatment cycle for 12 cycles or more, such as 16 cycles or more, such as 24 cycles or more, such as 36 cycles or more. In some embodiments, the 21-day treatment cycle is administered for no more than 3, no more than 4, no more than 5, or no more than 6 four-week treatment cycles. The number of treatment cycles suitable for any specific subject or group of subjects may be determined by a person of skill in the art, typically a physician. In some embodiments, the anti-CD20 antibody or antigen-binding fragment thereof as described herein is administered at a dose of 375 mg/m² of the subject's body surface area on day 1 of the first 21-day treatment cycle and at a dose of 1400 mg on day 1 of each 21-day treatment cycle thereafter. In some embodiments, the anti-CD20 antibody or antigen-binding fragment thereof as described herein is administered by intravenous infusion at a dose of 375 mg/m² of the subject's body surface area on day 1 of the first 21-day treatment cycle and is administered by subcutaneous injection at a dose of 1400 mg on day 1 of each 21-day treatment cycle thereafter. In some embodiments, the anti-CD20 antibody or antigen-binding fragment thereof as described herein is administered by intravenous infusion at a dose of 375 mg/m² of the subject's body surface area on day 1 of the first 21-day treatment cycle and is administered by subcutaneous injection at a dose of 1400 mg on day 1 of each 21-day treatment cycle thereafter, wherein the anti-CD20 antibody or antigen-binding fragment thereof is rituximab or a biosimilar thereof. In some embodiments, the anti-CD20 antibody or antigen-binding fragment thereof as described herein is administered by intravenous infusion at a dose of 375 mg/m² of the subject's body surface area on day 1 of the first 21-day treatment cycle and is administered by subcutaneous injection at a dose of 1400 mg on day 1 of each 21-day treatment cycle thereafter, wherein the anti-CD20 antibody or antigen-binding fragment thereof is rituximab.

In some embodiments, the dose of the anti-CD30 antibody-drug conjugate described herein is 1.2 mg/kg and is administered once about every 3 weeks (e.g., ±3 days), the dose of lenalidomide, or salt or solvate thereof, is 20 mg and is administered once about every day, and the dose of the anti-CD20 antibody or antigen-binding fragment thereof as described herein is 375 mg/m² of the subject's body surface and is administered once about every 3 weeks (e.g., ±3 days). In some embodiments, the dose of the anti-CD30 antibody-drug conjugate described herein is 1.2 mg/kg and is administered once every 3 weeks, the dose of lenalidomide, or salt or solvate thereof, is 20 mg and is administered once every day, and the dose of the anti-CD20 antibody or antigen-binding fragment thereof as described herein is 375 mg/m² of the subject's body surface and is administered once every 3 weeks. In some embodiments, the dose of the anti-CD30 antibody-drug conjugate is 1.2 mg/kg and is administered once every 3 weeks and the antibody-drug conjugate is brentuximab vedotin, the dose of lenalidomide, or salt or solvate thereof, is 20 mg and is administered once every day and the dose of the anti-CD20 antibody or antigen-binding fragment is 375 mg/m² of the subject's body surface and is administered once every 3 weeks and the anti-CD20 antibody or antigen-binding fragment is rituximab. In some embodiments, the anti-CD30 antibody-drug conjugate described herein is administered by intravenous infusion. In some embodiments, the lenalidomide, or salt or solvate thereof, is administered orally. In some embodiments, the anti-CD20 antibody or antigen-binding fragment thereof as described herein is administered by intravenous infusion.

In some embodiments, the dose of the anti-CD30 antibody-drug conjugate described herein is 0.9 mg/kg and is administered once about every 3 weeks (e.g., ±3 days), the dose of lenalidomide, or salt or solvate thereof, is 20 mg and is administered once about every day, and the dose of the anti-CD20 antibody or antigen-binding fragment thereof as described herein is 375 mg/m² of the subject's body surface and is administered once about every 3 weeks (e.g., ±3 days). In some embodiments, the dose of the anti-CD30 antibody-drug conjugate described herein is 0.9 mg/kg and is administered once every 3 weeks, the dose of lenalidomide, or salt or solvate thereof, is 20 mg and is administered once every day, and the dose of the anti-CD20 antibody or antigen-binding fragment thereof as described herein is 375 mg/m² of the subject's body surface and is administered once every 3 weeks. In some embodiments, the dose of the anti-CD30 antibody-drug conjugate is 0.9 mg/kg and is administered once every 3 weeks and the antibody-drug conjugate is brentuximab vedotin, the dose of lenalidomide, or salt or solvate thereof, is 20 mg and is administered once every day and the dose of the anti-CD20 antibody or antigen-binding fragment is 375 mg/m² of the subject's body surface and is administered once every 3 weeks and the anti-CD20 antibody or antigen-binding fragment is rituximab. In some embodiments, the anti-CD30 antibody-drug conjugate described herein is administered by intravenous infusion. In some embodiments, the lenalidomide, or salt or solvate thereof, is administered orally. In some embodiments, the anti-CD20 antibody or antigen-binding fragment thereof as described herein is administered by intravenous infusion.

In some embodiments, the dose of the anti-CD30 antibody-drug conjugate described herein is 1.2 mg/kg and is administered once about every 3 weeks (e.g., ±3 days), the dose of lenalidomide, or salt or solvate thereof, is 20 mg and is administered once about every day, and the dose of the anti-CD20 antibody or antigen-binding fragment thereof as described herein is 1400 mg and is administered once about every 3 weeks (e.g., ±3 days). In some embodiments, the dose of the anti-CD30 antibody-drug conjugate described herein is 1.2 mg/kg and is administered once every 3 weeks, the dose of lenalidomide, or salt or solvate thereof, is 20 mg and is administered once every day, and the dose of the anti-CD20 antibody or antigen-binding fragment thereof as described herein is 1400 mg and is administered once every 3 weeks. In some embodiments, the dose of the anti-CD30 antibody-drug conjugate is 1.2 mg/kg and is administered once every 3 weeks and the antibody-drug conjugate is brentuximab vedotin, the dose of lenalidomide, or salt or solvate thereof, is 20 mg and is administered once every day and the dose of the anti-CD20 antibody or antigen-binding fragment is 1400 mg and is administered once every 3 weeks and the anti-CD20 antibody or antigen-binding fragment is rituximab. In some embodiments, the anti-CD30 antibody-drug conjugate described herein is administered by intravenous infusion. In some embodiments, the lenalidomide, or salt or solvate thereof, is administered orally. In some embodiments, the anti-CD20 antibody or antigen-binding fragment thereof as described herein is administered by subcutaneous injection.

In some embodiments, the dose of the anti-CD30 antibody-drug conjugate described herein is 0.9 mg/kg and is administered once about every 3 weeks (e.g., ±3 days), the dose of lenalidomide, or salt or solvate thereof, is 20 mg and is administered once about every day, and the dose of the anti-CD20 antibody or antigen-binding fragment thereof as described herein is 1400 mg and is administered once about every 3 weeks (e.g., ±3 days). In some embodiments, the dose of the anti-CD30 antibody-drug conjugate described herein is 0.9 mg/kg and is administered once every 3 weeks, the dose of lenalidomide, or salt or solvate thereof, is 20 mg and is administered once every day, and the dose of the anti-CD20 antibody or antigen-binding fragment thereof as described herein is 1400 mg and is administered once every 3 weeks. In some embodiments, the dose of the anti-CD30 antibody-drug conjugate is 0.9 mg/kg and is administered once every 3 weeks and the antibody-drug conjugate is brentuximab vedotin, the dose of lenalidomide, or salt or solvate thereof, is 20 mg and is administered once every day and the dose of the anti-CD20 antibody or antigen-binding fragment is 1400 mg and is administered once every 3 weeks and the anti-CD20 antibody or antigen-binding fragment is rituximab. In some embodiments, the anti-CD30 antibody-drug conjugate described herein is administered by intravenous infusion. In some embodiments, the lenalidomide, or salt or solvate thereof, is administered orally. In some embodiments, the anti-CD20 antibody or antigen-binding fragment thereof as described herein is administered by subcutaneous injection.

In some embodiments, the dose of the anti-CD30 antibody-drug conjugate described herein is 1.2 mg/kg and is administered on day 1 of a 21-day treatment cycle, the dose of lenalidomide, or salt or solvate thereof, is 20 mg and is administered once every day, the first dose of the anti-CD20 antibody or antigen-binding fragment thereof as described herein is 375 mg/m² of the subject's body surface area and is administered on day 1 of the first 21-day treatment cycle, and the anti-CD20 antibody or antigen-binding fragment thereof as described herein is administered at a dose of 1400 mg on day 1 of each 21-day treatment cycle thereafter. In some embodiments, the dose of the anti-CD30 antibody-drug conjugate described herein is 1.2 mg/kg and is administered on day 1 of a 21-day treatment cycle and the antibody-drug conjugate is brentuximab vedotin, the dose of lenalidomide, or salt or solvate thereof, is 20 mg and is administered once every day, the first dose of the anti-CD20 antibody or antigen-binding fragment thereof as described herein is 375 mg/m² of the subject's body surface area and is administered on day 1 of the first 21-day treatment cycle, and the anti-CD20 antibody or antigen-binding fragment thereof as described herein is administered at a dose of 1400 mg on day 1 of each 21-day treatment cycle thereafter and the anti-CD20 antibody or antigen-binding fragment is rituximab. In some embodiments, the anti-CD30 antibody-drug conjugate described herein is administered by intravenous infusion. In some embodiments, the lenalidomide, or salt or solvate thereof, is administered orally. In some embodiments, the anti-CD20 antibody or antigen-binding fragment thereof as described herein is administered by intravenous infusion on day 1 of the first 21-day treatment cycle and is administered by subcutaneous injection on day 1 of each 21-day treatment cycle thereafter.

In some embodiments, the dose of the anti-CD30 antibody-drug conjugate described herein is 0.9 mg/kg and is administered on day 1 of a 21-day treatment cycle, the dose of lenalidomide, or salt or solvate thereof, is 20 mg and is administered once every day, the first dose of the anti-CD20 antibody or antigen-binding fragment thereof as described herein is 375 mg/m² of the subject's body surface area and is administered on day 1 of the first 21-day treatment cycle, and the anti-CD20 antibody or antigen-binding fragment thereof as described herein is administered at a dose of 1400 mg on day 1 of each 21-day treatment cycle thereafter. In some embodiments, the dose of the anti-CD30 antibody-drug conjugate described herein is 0.9 mg/kg and is administered on day 1 of a 21-day treatment cycle and the antibody-drug conjugate is brentuximab vedotin, the dose of lenalidomide, or salt or solvate thereof, is 20 mg and is administered once every day, the first dose of the anti-CD20 antibody or antigen-binding fragment thereof as described herein is 375 mg/m² of the subject's body surface area and is administered on day 1 of the first 21-day treatment cycle, and the anti-CD20 antibody or antigen-binding fragment thereof as described herein is administered at a dose of 1400 mg on day 1 of each 21-day treatment cycle thereafter and the anti-CD20 antibody or antigen-binding fragment is rituximab. In some embodiments, the anti-CD30 antibody-drug conjugate described herein is administered by intravenous infusion. In some embodiments, the lenalidomide, or salt or solvate thereof, is administered orally. In some embodiments, the anti-CD20 antibody or antigen-binding fragment thereof as described herein is administered by intravenous infusion on day 1 of the first 21-day treatment cycle and is administered by subcutaneous injection on day 1 of each 21-day treatment cycle thereafter.

### G. Treatment Outcome

In one aspect, a method of treating non-Hodgkin lymphoma, such as DLBCL, with an anti-CD30 antibody-drug conjugate or antigen-binding fragment thereof as described herein and/or lenalidomide, or salt or solvate thereof, and/or an anti-CD20 antibody or antigen-binding fragment thereof as described herein results in an improvement in one or more therapeutic effects in the subject after administration of the antibody-drug conjugate and/or lenalidomide, or salt or solvate thereof, and/or the anti-CD20 antibody or antigen-binding fragment thereof relative to a baseline.

In some embodiments, the one or more therapeutic effects is the objective response rate, the duration of response, the time to response, progression free survival, overall survival, or any combination thereof. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment is assessed using the Lugano Classification Revised Staging System for nodal non-Hodgkin and Hodgkin lymphomas as described in Cheson B D, et al. J Clin Oncol. 32(27):3059-68 (2014). In some embodiments, the criteria for response assessment is as described in the following table:

Revised Criteria for Response Assessment

| Response and Site | PET-CT-Based Response | CT-Based Response |
|---|---|---|
| Complete | Complete meabolic response | Complete radiologic response (all of the |
| | | following) |
| Lymph nodes and | Score 1, 2, or 3* with or without a | Target nodes/nodal masses must regress |
| extralymphatic sites | residual mass on 5 [symbol] | to ≤ 1.5 cm in LDi |
| | It is recognized that in Waldeyer's ring | No extralymphatic sites of disease |
| | or extranodal sites with high | |
| | physiologic uptake or with activation | |
| | within spleen or marrow (eg, with | |
| | chemotherapy or myeloid colony- | |
| | stimulating factors), uptake may be | |
| | greater than normal mediastinum | |
| | and/or liver. In this circumstance, | |
| | complete metabolic response may be | |
| | inferred if uptake at sites of initial | |
| | involvement is no greater than | |
| | surrounding normal tissue even if the | |
| | tissue has high physiologic uptake | |
| Nonmeasured lesion | Not applicable | Absent |
| Organ enlargement | Not applicable | Regress to normal |
| New lesions | None | None |
| Bone marrow | No evidence of FDG-avid disease in | Normal by morphology; if indeterminate, |
| | marrow | IHC negative |
| Partial | Partial metabolic response | Partial remission (all of the following) |
| Lymph nodes and | Score 4 or 5** with reduced uptake | ≥50% decrease in SPD of up to 6 target |
| extralymphatic sites | compared with baseline and residual | measurable nodes and extranodal sites |
| | mass(es) of any size | |
| | At interim, these findings suggest | When a lesion is too small to measure on |
| | responding disease | CT, assign 5 mm × 5 mm as the default |
| | | value |
| | At end of treatment, these findings | When no longer visible, 0 × 0 mm |
| | indicate residual disease | For a node > 5 mm × 5 mm, but smaller |
| | | than normal, use actual measurement for |
| | | calculation |
| Nonmeasured lesion | Not applicable | Absent/normal, regressed, but no increase |
| Organ enlargement | Not applicable | Spleen must have regressed by > 50% in |
| | | length beyond normal |
| New lesions | None | None |
| Bone marrow | Residual uptake higher than uptake in | Not applicable |
| | normal marrow but reduced compared | |
| | with baseline (diffuse uptake | |
| | compatible with reactive changes from | |
| | chemotherapy allowed). If there are | |
| | persistent focal changes in the marrow | |
| | in the context of a nodal response, | |
| | consideration should be given to | |
| | further evaluation with MRI or biopsy | |
| | or an interval scan | |
| No response or stable | No metabolic response | Stable disease |
| disease | | |
| Target nodes/nodal | Score 4 or 5 with no significant change | <50% decrease from baseline in SPD of up |
| masses, extranodal | in FDG uptake from baseline at interim | to 6 dominant, measurable nodes and |
| lesions | or end of treatment | extranodal sites; no criteria for progressive |
| | | disease are met |
| Nonmeasured lesion | Not applicable | No increase consistent with progression |
| Organ enlargement | Not applicable | No increase consistent with progression |
| New lesions | None | None |
| Bone marrow | No change from baseline | Not applicable |
| Progressive disease | Progressive metabolic disease | Progressive disease requires at least 1 of |
| | | the following |
| Individual target | Score 4 or 5 with an increase in | PPD progression: |
| nodes/nodal masses | intensity of uptake from baseline | |
| | and/or | |
| Extranodal | New FDG-avid foci consistent with | An individual node/lesion must be |
| lesions | lymphoma at interim or end-of- | abnormal with: |
| | treatment assessment | LDi > 1.5 cm and |
| | | Increase by ≥ 50% from PPD nadir and |
| | | An increase in LDi or SDi from nadir |
| | | 0.5 cm for lesions ≤ 2 cm |
| | | 1.0 cm for lesions > 2 cm |
| | | In the setting of splenomegaly, the splenic |
| | | length must increase by > 50% of the extent |
| | | of its prior increase beyond baseline (eg, a |
| | | 15-cm spleen must increase to > 16 cm). If |
| | | no prior splenomegaly, must increase by at |
| | | least 2 cm from baseline |
| | | New or recurrent splenomegaly |
| Nonmeasured lesion | None | New or clear progression of preexisting |
| | | nonmeasured lesions |
| New lesions | New FDG-avid foci consistent with | Regrowth of previously resolved lesions |
| | lymphoma rather than another etiology | A new node > 1.5 cm in any axis |
| | (eg, infection, inflammation). If | A new extranodal site > 1.0 cm in any axis; |
| | uncertain regarding etiology of new | if < 1.0 cm in any axis, its presence must be |
| | lesions, biopsy or interval scan may be | unequivocal and must be attributable to |
| | considered | lymphoma |
| | | Assessable disease of any size |
| | | unequivocally attributable to lymphoma |
| Bone marrow | New or recurrent FDG-avid foci | New or recurrent involvement |

Abbreviations: 5PS, 5-point scale; CT, computed tomography; FDG, fluorodeoxyglucose; IHC, immunohistochemistry; LDi, longest transverse diameter of a lesion; MRI, magnetic resonance imaging; PET, positron emission tomography; PPD, cross product of the LDi and perpendicular diameter; SDi, shortest axis perpendicular to the LDi; SPD, sum of the product of the perpendicular diameters for multiple lesions.
*A score of 3 in many patients indicates a good prognosis with standard treatment, especially if at the time of an interim scan. However, in trials involving PET where de-escalation is investigated, it may be preferable to consider a score of 3 as inadequate response (to avoid undertreatment). Measured dominant lesions: Up to six of the largest dominant nodes, nodal masses, and extranodal lesions selected to be clearly measurable in two diameters. Nodes should preferably be from disparate regions of the body and should include, where applicable, mediastinal and retroperitoneal areas. Non-nodal lesions include those in solid organs (eg, liver, spleen, kidneys, lungs), GI involvement, cutaneous lesions, or those noted on palpation. Nonmeasured lesions: Any disease not selected as measured, dominant disease and truly assessable disease should be considered not measured. These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer's ring or in extranodal sites (eg, GI tract, liver, bone marrow), FDG uptake may be greater than in the mediastinum with complete metabolic response, but should be no higher than surrounding normal physiologic uptake (eg, with marrow activation as a result of chemotherapy or myeloid growth factors).
**PET 5PS: 1, no uptake above background; 2, uptake ≤ mediastinum; 3, uptake > mediastinum but ≤ liver; 4, uptake moderately > liver; 5, uptake markedly higher than liver and/or new lesions; X, new areas of uptake unlikely to be related to lymphoma. [symbol]
indicates data missing or illegible when filed

In one embodiment of the methods or uses or product for uses provided herein, the effectiveness of treatment with an anti-CD30 antibody-drug conjugate or antigen-binding fragment thereof as described herein and/or lenalidomide, or salt or solvate thereof, and/or an anti-CD20 antibody or antigen-binding fragment thereof as described herein is assessed by measuring the objective response rate. In some embodiments, the objective response rate is the proportion of patients with tumor size reduction of a predefined amount and for a minimum period of time. In some embodiments the objective response rate is based upon Cheson criteria. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses described herein, response to treatment with an anti-CD30 antibody-drug conjugate or antigen-binding fragment thereof as described herein and/or lenalidomide, or salt or solvate thereof, and/or an anti-CD20 antibody or antigen-binding fragment thereof as described herein is assessed by measuring the time of progression free survival after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits progression-free survival of at least about 6 months after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits progression-free survival of at least about one year after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits progression-free survival of at least about two years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits progression-free survival of at least about three years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits progression-free survival of at least about four years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits progression-free survival of at least about five years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits progression-free survival of at least 6 months after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits progression-free survival of at least one year after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits progression-free survival of at least two years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits progression-free survival of at least three years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits progression-free survival of at least four years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits progression-free survival of at least five years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein.

In one embodiment of the methods or uses or product for uses described herein, response to treatment with an anti-CD30 antibody-drug conjugate or antigen-binding fragment thereof as described herein and/or lenalidomide, or salt or solvate thereof, and/or an anti-CD20 antibody or antigen-binding fragment thereof as described herein is assessed by measuring the time of overall survival after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits overall survival of at least about 6 months after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits overall survival of at least about one year after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits overall survival of at least about two years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits overall survival of at least about three years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits overall survival of at least about four years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits overall survival of at least about five years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least about 12 months, at least eighteen months, at least two years, at least three years, at least four years, or at least five years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits overall survival of at least 6 months after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits overall survival of at least one year after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits overall survival of at least two years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits overall survival of at least three years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits overall survival of at least four years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the subject exhibits overall survival of at least five years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein.

In one embodiment of the methods or uses or product for uses described herein, response to treatment with an anti-CD30 antibody-drug conjugate or antigen-binding fragment thereof as described herein and/or lenalidomide, or salt or solvate thereof, and/or an anti-CD20 antibody or antigen-binding fragment thereof as described herein is assessed by measuring the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein is at least about 6 months after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein is at least about one year after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein is at least about two years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein is at least about three years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein is at least about four years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein is at least about five years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein is at least 6 months after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein is at least one year after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein is at least two years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein is at least three years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein is at least four years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein. In some embodiments, the duration of response to the anti-CD30 antibody-drug conjugate or antigen-binding fragment thereof as described herein and/or lenalidomide, or salt or solvate thereof, and/or the anti-CD20 antibody or antigen-binding fragment thereof as described herein is at least five years after administration of the anti-CD30 antibody-drug conjugate or antigen-binding fragment thereof as described herein and/or lenalidomide, or salt or solvate thereof, and/or the anti-CD20 antibody or antigen-binding fragment thereof as described herein.

In some embodiments of the methods or uses or product for uses described herein, administering an anti-CD30 antibody-drug conjugate or antigen-binding fragment thereof as described herein and/or lenalidomide, or salt or solvate thereof, and/or an anti-CD20 antibody or antigen-binding fragment thereof as described herein to a subject results in a depletion of cancer cells, such as DLBCL cells, in the subject. In some embodiments, administering an anti-CD30 antibody-drug conjugate or antigen-binding fragment thereof as described herein and/or lenalidomide, or salt or solvate thereof, and/or an anti-CD20 antibody or antigen-binding fragment thereof as described herein results in a depletion of cancer cells by at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or about 100% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least about 5% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least about 10% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least about 20% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least about 30% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least about 40% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least about 50% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least about 60% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least about 70% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least about 80% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least about 90% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least about 95% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least about 99% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by about 100% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, administering an anti-CD30 antibody-drug conjugate or antigen-binding fragment thereof as described herein and/or lenalidomide, or salt or solvate thereof, and/or an anti-CD20 antibody or antigen-binding fragment thereof as described herein results in a depletion of cancer cells by at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least about 80%, at least about 90%, at least 95%, or 100% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least 5% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least 10% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least 20% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least 30% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least 40% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least 50% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least 60% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least 70% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least 80% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least 90% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least 95% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by at least 99% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject. In some embodiments, the cancer cells are depleted by 100% compared to the amount of cancer cells before administering the anti-CD30 antibody-drug conjugate or antigen-binding fragment thereof as described herein and/or lenalidomide, or salt or solvate thereof, and/or the anti-CD20 antibody or antigen-binding fragment thereof as described herein to the subject.

### III. Compositions

In some aspects, also provided herein are compositions (e.g., pharmaceutical compositions and therapeutic formulations) comprising any of the anti-CD30 antibody-drug conjugate or antigen-binding fragment thereof as described herein and/or lenalidomide, or salt or solvate thereof, and/or the anti-CD20 antibody or antigen-binding fragment thereof as described herein.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000).

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%- 1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intramolecular interactions. Tonicity agents can be present in any amount between about 0.1% to about 25% by weight or between about 1% to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In some embodiments provided herein, a formulation comprising the anti-CD30 antibody-drug conjugate or antigen-binding fragment thereof as described herein and/or lenalidomide, or salt or solvate thereof, and/or the anti-CD20 antibody or antigen-binding fragment thereof as described herein thereof described herein does not comprise a surfactant (i.e., is free of surfactant).

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The invention provides compositions comprising a population of anti-CD30 antibody-drug conjugates or antigen-binding fragments thereof as described herein for use in a method of treating non-Hodgkin lymphoma as described herein. In some aspects, provided herein are compositions comprising a population of antibody-drug conjugates, wherein the antibody-drug conjugates comprise a linker attached to MMAE, wherein the antibody-drug conjugate has the following structure:

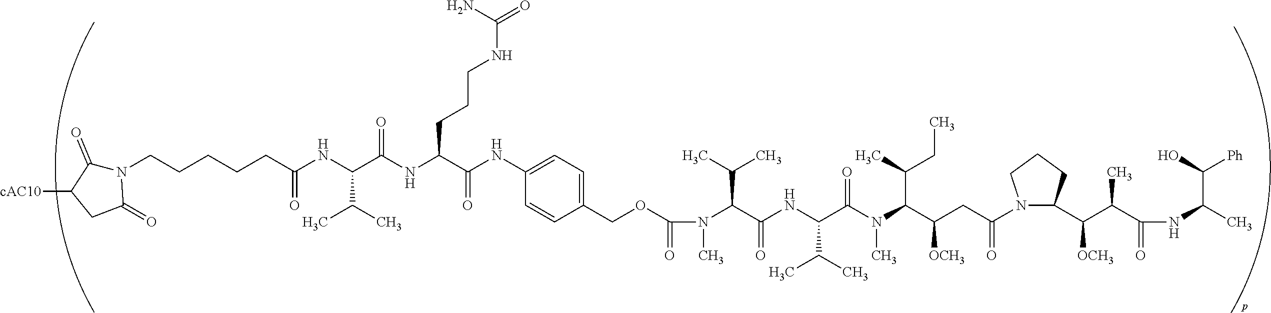

wherein p denotes a number from 1 to 8, e.g., 1, 2, 3, 4, 5, 6, 7 or 8, and cAC10 designates the anti-CD30 antibody brentuximab. In some embodiments, p denotes a number from 3 to 5. In some embodiments, the average value of p in the composition is about 4. In some embodiments, the population is a mixed population of antibody-drug conjugates in which p varies from 1 to 8 for each antibody-drug conjugate. In some embodiments, the population is a homogenous population of antibody-drug conjugates with each antibody-drug conjugate having the same value for p.

In some embodiments, a composition comprising an anti-CD30 antibody-drug conjugate or antigen-binding fragment thereof as described herein is coadministered with a composition comprising lenalidomide, or salt or solvate thereof, and/or a composition comprising an anti-CD20 antibody or antigen-binding fragment thereof as described herein. In some embodiments the coadministration is simultaneous or sequential. In some embodiments, the anti-CD30 antibody-drug conjugate as described herein is administered simultaneously with lenalidomide, or salt or solvate thereof, and/or the anti-CD20 antibody or antigen-binding fragment thereof as described herein. In some embodiments, simultaneous means that the anti-CD30 antibody-drug conjugate described herein and lenalidomide, or salt or solvate thereof, and/or the anti-CD20 antibody or antigen-binding fragment thereof as described herein are administered to the subject less than about one hour apart, such as less than about 30 minutes apart, less than about 15 minutes apart, less than about 10 minutes apart or less than about 5 minutes apart. In some embodiments, simultaneous means that the anti-CD30 antibody-drug conjugate described herein and lenalidomide, or salt or solvate thereof, and/or the anti-CD20 antibody or antigen-binding fragment thereof as described herein are administered to the subject less than one hour apart, such as less than 30 minutes apart, less than 15 minutes apart, less than 10 minutes apart or less than 5 minutes apart. In some embodiments, the anti-CD30 antibody-drug conjugate described herein is administered sequentially with lenalidomide, or salt or solvate thereof, and/or the anti-CD20 antibody or antigen-binding fragment thereof as described herein. In some embodiments, sequential administration means that the anti-CD30 antibody-drug conjugate described herein and lenalidomide, or salt or solvate thereof, and/or the anti-CD20 antibody or antigen-binding fragment thereof as described herein are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, the anti-CD30 antibody-drug conjugate described herein is administered after the administration of lenalidomide, or salt or solvate thereof, and/or the anti-CD20 antibody or antigen-binding fragment thereof as described herein. In some embodiments, the anti-CD30 antibody-drug conjugate described herein is administered at least 30 minutes after the administration of lenalidomide, or salt or solvate thereof, and/or the anti-CD20 antibody or antigen-binding fragment thereof as described herein. In some embodiments, the anti-CD30 antibody-drug conjugate described herein is administered 30 minutes after the administration of lenalidomide, or salt or solvate thereof, and/or the anti-CD20 antibody or antigen-binding fragment thereof as described herein. In some embodiments, the anti-CD30 antibody-drug conjugate described herein is administered about 30 minutes to about 3 hours after the administration of lenalidomide, or salt or solvate thereof, and/or the anti-CD20 antibody or antigen-binding fragment thereof as described herein. In some embodiments, the anti-CD30 antibody-drug conjugate described herein is administered about 30 minutes to about 2 hours after the administration of lenalidomide, or salt or solvate thereof, and/or the anti-CD20 antibody or antigen-binding fragment thereof as described herein. In some embodiments, the anti-CD30 antibody-drug conjugate described herein is administered about 30 minutes to about 60 minutes after the administration of the lenalidomide, or salt or solvate thereof, and/or the anti-CD20 antibody or antigen-binding fragment thereof as described herein. In some embodiments, the anti-CD30 antibody-drug conjugate described herein is administered 30 minutes to 3 hours after the administration of lenalidomide, or salt or solvate thereof, and/or the anti-CD20 antibody or antigen-binding fragment thereof as described herein. In some embodiments, the anti-CD30 antibody-drug conjugate described herein is administered 30 minutes to 2 hours after the administration of lenalidomide, or salt or solvate thereof, and/or the anti-CD20 antibody or antigen-binding fragment thereof as described herein. In some embodiments, the anti-CD30 antibody-drug conjugate described herein is administered 30 minutes to 60 minutes after the administration of lenalidomide, or salt or solvate thereof, and/or the anti-CD20 antibody or antigen-binding fragment thereof as described herein. In some embodiments, the anti-CD30 antibody-drug conjugate described herein is administered about 30 minutes to about 120 minutes before the administration of the anti-CD20 antibody or antigen-binding fragment thereof as described herein. In some embodiments, the anti-CD30 antibody-drug conjugate described herein is administered about 60 minutes to about 90 minutes before the administration of the anti-CD20 antibody or antigen-binding fragment thereof as described herein. In some embodiments, the anti-CD30 antibody-drug conjugate described herein is administered 60 minutes to 90 minutes before the administration of the anti-CD20 antibody or antigen-binding fragment thereof as described herein. In some embodiments, the anti-CD30 antibody-drug conjugate described herein is administered about 60 minutes before the administration of the anti-CD20 antibody or antigen-binding fragment thereof as described herein. In some embodiments, the anti-CD30 antibody-drug conjugate described herein is administered about 90 minutes before the administration of the anti-CD20 antibody or antigen-binding fragment thereof as described herein. In some embodiments, the anti-CD30 antibody-drug conjugate described herein is administered 60 minutes before the administration of the anti-CD20 antibody or antigen-binding fragment thereof as described herein. In some embodiments, the anti-CD30 antibody-drug conjugate described herein is administered 90 minutes before the administration of the anti-CD20 antibody or antigen-binding fragment thereof as described herein.

In some embodiments, a composition comprising an ant-CD30 antibody-drug conjugate as described herein and/or lenalidomide, or salt or solvate thereof, and/or an anti-CD20 antibody or antigen-binding fragment thereof as described herein is coadministered with one or more therapeutic agents to eliminate or reduce the severity of one or more adverse events. In some embodiments, a composition comprising an anti-CD30 antibody-drug conjugate as described herein and/or lenalidomide, or salt or solvate thereof, and/or an anti-CD20 antibody or antigen-binding fragment thereof as described herein is coadministered with one or more therapeutic agents to prevent the development of the adverse event or to reduce the severity of the adverse event.

In some embodiments, a composition comprising an anti-CD30 antibody-drug conjugate as described herein and/or lenalidomide, or salt or solvate thereof, and/or an anti-CD20 antibody or antigen-binding fragment thereof as described herein is coadministered with one or additional therapeutic agents. In some embodiments the coadministration is simultaneous or sequential. In some embodiments, the anti-CD30 antibody-drug conjugate as described herein and/or lenalidomide, or salt or solvate thereof, and/or the anti-CD20 antibody or antigen-binding fragment thereof as described herein is administered simultaneously with the one or more additional therapeutic agents. In some embodiments, simultaneous means that the anti-CD30 antibody-drug conjugate described herein and/or lenalidomide, or salt or solvate thereof, and/or the anti-CD20 antibody or antigen-binding fragment thereof as described herein and the one or more therapeutic agents are administered to the subject less than about one hour apart, such as less than about 30 minutes apart, less than about 15 minutes apart, less than about 10 minutes apart or less than about 5 minutes apart. In some embodiments, simultaneous means that the anti-CD30 antibody-drug conjugate described herein and/or lenalidomide, or salt or solvate thereof, and/or the anti-CD20 antibody or antigen-binding fragment thereof as described herein and the one or more therapeutic agents are administered to the subject less than one hour apart, such as less than 30 minutes apart, less than 15 minutes apart, less than 10 minutes apart or less than 5 minutes apart. In some embodiments, the anti-CD30 antibody-drug conjugate described herein and/or lenalidomide, or salt or solvate thereof, and/or the anti-CD20 antibody or antigen-binding fragment thereof as described herein is administered sequentially with the one or more additional therapeutic agents. In some embodiments, sequential administration means that the anti-CD30 antibody-drug conjugate described herein and/or lenalidomide, or salt or solvate thereof, and/or the anti-CD20 antibody or antigen-binding fragment thereof as described herein and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart.

In some embodiments, a composition comprising an anti-CD30 antibody-drug conjugate as described herein and/or lenalidomide, or salt or solvate thereof, and/or an anti-CD20 antibody or antigen-binding fragment thereof as described herein is coadministered with one or more therapeutic agents to eliminate or reduce the severity of one or more adverse events. In some embodiments the coadministration is simultaneous or sequential. In some embodiments, the anti-CD30 antibody-drug conjugate described herein and/or lenalidomide, or salt or solvate thereof, and/or the anti-CD20 antibody or antigen-binding fragment thereof as described herein is administered simultaneously with the one or more therapeutic agents to eliminate or reduce the severity of one or more adverse events. In some embodiments, simultaneous means that the anti-CD30 antibody-drug conjugate described herein and/or lenalidomide, or salt or solvate thereof, and/or the anti-CD20 antibody or antigen-binding fragment thereof as described herein and the one or more therapeutic agents to eliminate or reduce the severity of one or more adverse events are administered to the subject less than about one hour apart, such as less than about 30 minutes apart, less than about 15 minutes apart, less than about 10 minutes apart or less than about 5 minutes apart. In some embodiments, simultaneous means that the anti-CD30 antibody-drug conjugate described herein and/or lenalidomide, or salt or solvate thereof, and/or the anti-CD20 antibody or antigen-binding fragment thereof as described herein and the one or more therapeutic agents to eliminate or reduce the severity of one or more adverse events are administered to the subject less than one hour apart, such as less than 30 minutes apart, less than 15 minutes apart, less than 10 minutes apart or less than 5 minutes apart. In some embodiments, the anti-CD30 antibody-drug conjugate described herein and/or lenalidomide, or salt or solvate thereof, and/or the anti-CD20 antibody or antigen-binding fragment thereof as described herein is administered sequentially with the one or more therapeutic agents to eliminate or reduce the severity of one or more adverse events. In some embodiments, sequential administration means that the anti-CD30 antibody-drug conjugate described herein and/or lenalidomide, or salt or solvate thereof, and/or the anti-CD20 antibody or antigen-binding fragment thereof as described herein and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, the anti-CD30 antibody-drug conjugate described herein and/or lenalidomide, or salt or solvate thereof, and/or the anti-CD20 antibody or antigen-binding fragment thereof as described herein is administered prior to the one or more therapeutic agents to eliminate or reduce the severity of one or more adverse events. In some embodiments, the one or more therapeutic agents to eliminate or reduce the severity of one or more adverse events is administered prior to the anti-CD30 antibody-drug conjugate described herein and/or lenalidomide, or salt or solvate thereof, and/or the anti-CD20 antibody or antigen-binding fragment thereof as described herein.

### IV. Articles of Manufacture and Kits

In another aspect, an article of manufacture or kit is provided which comprises an anti-CD30 antibody-drug conjugate or antigen-binding fragment thereof as described herein and/or lenalidomide, or salt or solvate thereof, and/or an anti-CD20 antibody or antigen-binding fragment thereof as described herein. The article of manufacture or kit may further comprise instructions for use of the anti-CD30 antibody-drug conjugate or antigen-binding fragment thereof as described herein and/or lenalidomide, or salt or solvate thereof, and/or the anti-CD20 antibody or antigen-binding fragment thereof as described herein in the methods of the invention. Thus, in certain embodiments, the article of manufacture or kit comprises instructions for the use of an anti-CD30 antibody-drug conjugate or antigen-binding fragment thereof as described herein and/or lenalidomide, or salt or solvate thereof, and/or an anti-CD20 antibody or antigen-binding fragment thereof as described herein in methods for treating non-Hodgkin lymphoma in a subject comprising administering to the subject an effective amount of an anti-CD30 antibody-drug conjugate or antigen-binding fragment thereof as described herein and/or lenalidomide, or salt or solvate thereof, and/or an anti-CD20 antibody or antigen-binding fragment thereof as described herein. In some embodiments, the non-Hodgkin lymphoma is DLBCL. In some embodiments, the non-Hodgkin lymphoma is an advanced stage non-Hodgkin lymphoma. In some embodiments, the non-Hodgkin lymphoma is relapsed non-Hodgkin lymphoma. In some embodiments, the non-Hodgkin lymphoma is refractory non-Hodgkin lymphoma. In some embodiments, the subject is a human.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating non-Hodgkin lymphoma in a subject, such as DLBCL as described herein. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein the anti-CD30 antibody-drug conjugate is a first medicament, and which article or kit further comprises instructions on the label or package insert for treating the subject with the second medicament, in an effective amount. In some embodiments, the second medicament is lenalidomide, or salt or solvate thereof. In some embodiments, the label or package insert indicates that the first and second medicaments are to be administered sequentially or simultaneously, as described herein.

The article of manufacture or kit herein optionally further comprises a container comprising a third medicament, wherein the anti-CD30 antibody-drug conjugate is a first medicament, lenalidomide, or salt or solvate thereof, is a second medicament, and which article or kit further comprises instructions on the label or package insert for treating the subject with the third medicament, in an effective amount., In some embodiments, the third medicament is an anti-CD20 antibody or antigen-binding fragment thereof as described herein. In some embodiments, the label or package insert indicates that the first and/or second medicament and/or third are to be administered sequentially or simultaneously, as described herein.

In some embodiments, the anti-CD30 antibody-drug conjugate or antigen-binding fragment thereof as described herein and/or lenalidomide, or salt or solvate thereof, and/or the anti-CD20 antibody or antigen-binding fragment thereof as described herein is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: A Phase I Trial of Brentuximab Vedotin in Combination with Lenalidomide in Relapsed or Refractory Diffuse Large B-cell Lymphoma

This trial was an open-label phase I study in two parts. The maximum tolerated dose (MTD) and dose-limiting toxicities (DLTs) of the combination of brentuximab vedotin and lenalidomide were determined in a dose-escalation phase, followed by a dose-expansion phase where patients with relapsed or refractory (rel/ref) CD30-positive and CD30-negative DLBCL were treated at the MTD. “Positive” CD30 expression is defined as ≥1% staining on the malignant cells.

Brentuximab vedotin was administered on Day 1 (±1 day) of each 21-day cycle. The dose of brentuximab vedotin was administered by outpatient IV infusion given over approximately 30 minutes.

Lenalidomide was taken by mouth once a day on Days 1 through 21 of each 21-day cycle (except at Dose Level -2). Lenalidomide could be taken with or without food. Subjects were instructed to swallow the capsule whole with water and without being opened, broken, or chewed.

The maximum tolerated dose (MTh) was defined as the dose level immediately below the dose level at which 2 patients of a cohort (of 2 to 6 patients) experienced dose-limiting toxicity during the first cycle. Dose escalations proceeded until the MTD was reached.

Hematologic dose limiting toxicity (DLT) was defined as any of the following that occured during the first cycle that were attributed as possibly, probably, or definitely related to the study treatment:
Grade 4 neutropenia >7 day duration
Grade 4 infection with grade 3 or 4 neutropenia (life-threatening sepsis)
Grade 4 thrombocytopenia associated with life-threatening bleeding or requiring more than 1 transfusion
Treatment delay >14 days due to hematologic toxicity

Non-hematologic DLT was defined as any possibly, probably, or definitely related grade 3 or grade 4 non-hematologic toxicity that occured during the first cycle with the following specific exceptions:
Grade 3 or 4 nausea, vomiting, or anorexia that returns to Grade 1 prior to the start of Cycle 2
Grade 3 metabolic abnormalities (e.g., hyperglycemia)
Grade 3 fatigue

Dose escalations proceeded as follows after the occurrence of dose-limiting toxicity (DLT):

| Number of Patients | |
|---|---|
| with DLT at a | |
| Given Dose Level | Escalation Decision Rule |
| 0 out of 3 | Enter 3 patients at the next dose level. |
| ≥2 | Dose escalation will be stopped. Three (3) |
| | additional patients will be entered at the |
| | next lowest dose level if only 3 patients |
| | were treated previously at that dose. |
| 1 out of 3 | Enter at least 3 more patients at this dose |
| | level. |
| | If 0 of these 3 patients experience DLT, |
| | proceed to the next dose level. |
| | If 1 or more of this group suffer DLT, |
| | then dose escalation is stopped. Three (3) |
| | additional patients will be entered at the |
| | next lowest dose level if only 3 patients |
| | were treated previously at that dose. |
| ≤1 out of 6 at | This is generally the recommended phase 2 |
| highest dose level | dose. At least 6 patients must be entered at |
| below the maximally | the recommended phase 2 dose. |
| administered dose | |

Dose escalation occurred after all patients in the cohort completed the first cycle according to the following scheme:

Dose Escalation Schedule

| | Brentuximab | Lenalidomide |
|---|---|---|
| Dose Level | Vedotin Dose | Dose |
| Level −2 | 1.2 mg/kg | 15 mg |
| | | (D 1-14 only) |
| Level −1 | 1.2 mg/kg | 15 mg |
| Level 0 | 1.2 mg/kg | 20 mg |
| (Starting Dose) | | |
| Level 1 | 1.2 mg/kg | 25 mg |
| Level 2 | 1.8 mg/kg | 25 mg |

After determination of the MTD, a total of 15 CD30-positive (CD30 staining of malignant cells≥1% by visual assessment) and 22 CD30-negative (CD30 staining of malignant cells<1% by visual assessment) patients (including the patients treated at the MTD in the dose escalation phase) were enrolled into a dose-expansion cohort. The lenalidomide and BV doses were defined as the MTDs in the dose-escalation portion of the study; BV will be administered on Day 1 and lenalidomide on Days 1-21 of each 21-day cycle. The MTDs were found to be 1.2 mg/kg for BV and 20 mg for lenalidomide.

### Patient Selection

Inclusion Criteria
1. Relapsed or refractory de novo or transformed DLBCL disease following at least one prior systemic therapy (for DLBCL).
2. CD30 immunohistochemical staining using the anti-CD30 BerH2 antibody must be available on the most recent biopsy specimen. During dose escalation, patients could be either CD30 positive or CD30 negative. During dose expansion, at least 15 patients must have been CD30 positive and at least 15 patients must have been CD30 negative.
3. Post-autologous stem cell transplant (ASCT) or not a candidate for ASCT. Prior allogeneic stem cell transplant was allowed if patient was off all immunosuppressives and had no evidence of active graft-versus-host disease (GVHD).
4. Prior treatment with brentuximab vedotin was allowed provided the patient did not progress on BV or within 30 days of last dose of BV. Patients must have been at least 3 months from the last dose of BV.
5. Bidimensional measurable disease of at least 1.5 cm in the greatest transverse diameter as documented by CT or PET/CT.
6. At least 18 years of age.
7. ECOG performance status≤2 (see Appendix A)
8. Bone marrow and organ function as defined below:
   a. Absolute neutrophil count (ANC)≥1,000/mcl
   b. Platelets≥50,000/mcl
   c. Serum bilirubin≤1.5×institutional upper limit of normal (IULN) OR
   Serum bilirubin≤3.0×IULN for patients with Gilbert's disease or documented hepatic involvement with NHL
   d. Alanine aminotransferase (ALT) and aspartate aminotransferase (AST)≤2.5×IULN OR
   ALT and AST≤5.0×IULN for patients with documented hepatic involvement with NHL
   e. Creatinine clearance≥60 mL/min/1.73 m² as calculated by Cockcroft-Gault
9. Women of childbearing potential must have followed pregnancy testing requirements as outlined in the Revlimid REMS® program material. This was defined as either committing to continued abstinence from heterosexual intercourse or beginning TWO acceptable methods of contraception (one highly effective method and one additional effective method AT THE SAME TIME) at least 28 days prior to the start of lenalidomide, for the duration of study participation, and for 28 days following the last doses of brentuximab vedotin and lenalidomide. Women of childbearing potential must also have agreed to ongoing pregnancy testing. Men must have agreed to use a latex condom during sexual contact with a woman of childbearing potential even if they have had a successful vasectomy. All patients must have been counseled at a minimum of every 28 days about pregnancy precautions and risks of fetal exposure. If a woman became pregnant or suspected she was pregnant while participating in this study, she must have informed her treating physician immediately.
10. All study participants must have been registered into the mandatory Revlimid REMS® program and have been willing to comply with its requirements. Per standard Revlimid REMS® program requirements, all physicians who prescribed lenalidomide for research subjects enrolled into this trial, must have been registered in, and must complied with, all requirements of the Revlimid REMS® program.
11. Able to understand and willing to sign an IRB approved written informed consent document (or that of legally authorized representative, if applicable).

Exclusion Criteria
1. Primary mediastinal B-cell lymphoma
2. A history of other primary invasive malignancy that was in remission for at least 3 years or a current diagnosis of myelodysplastic syndrome (MDS) or an immature leukemia such as acute myeloid leukemia (AML).
3. Known active cerebral/meningeal lymphoma.
4. Present or history of progressive multifocal leukoencephalopathy (PML).
5. NYHA Class III or IV congestive heart failure.
6. Active CTCAE version 4.03 grade 3 or higher viral, bacterial, or fungal infection.
7. Known to be positive for hepatitis B by surface antigen expression and hepatitis B core antibody.
8. Known to have active hepatitis C infection (positive by polymerase chain reaction) or on antiviral therapy for hepatitis C within 6 months prior to the first doses of brentuximab vedotin and lenalidomide.

9. Known to be positive for HIV.

10. Receiving chemotherapy, radiotherapy, biologics, and/or other antitumor treatment with immunotherapy that was not completed at least 3 weeks prior to study entry, unless underlying disease was progressing on therapy.

11. Currently receiving any other investigational agents.

12. Known hypersensitivity to any excipient contained in the drug formulation of brentuximab vedotin or lenalidomide.

13. Pregnant and/or breastfeeding. Women of childbearing potential must have had a negative serum or urine pregnancy test with a sensitivity of at least 50 mIU/mL within 10-14 days prior to and again within 24 hours of starting lenalidomide.

14. Receiving immunosuppressive therapy.

15. Refractory to prior therapy with brentuximab vedotin (evidence of progression within 30 days of the last dose).

16. Prior therapy with lenalidomide.

### Dose Delays/Dose Modifications

If adverse events (AEs) were encountered attributable to lenalidomide, dose reductions were allowed in 5 mg increments to a lowest permissible dose of 5 mg daily. If AEs were encountered attributable to brentuximab vedotin, dose reduction to 0.9 mg/kg was allowed. Doses of brentuximab vedotin and lenalidomide were not re-escalated after a dose reduction. The only exception was the Cycle 2 dose of lenalidomide, which could be re-escalated with use of growth factors if the Cycle 1 dose was reduced due to neutropenia.

The start of the next cycle could be delayed for up to 3 weeks if additional time was required for the patient to recover from study treatment associated toxicity experienced during the current cycle.

Brentuximab vedotin dose could be delayed or modified according to the following tables:

| Starting dose | Reduction 1 | Reduction 2 |
|---|---|---|
| 1.8 mg/kg | 1.2 mg/kg | 0.9 mg/kg |
| 1.2 mg/kg | 0.9 mg/kg | Off study |

| NCI CTC | |
|---|---|
| Toxicity Grade | Dose Modification Instructions |
| Grade 3 or 4 | Hold brentuximab vedotin until toxicity resolves to ≤ grade 2. |
| neutropenia | Follow CBC weekly. |
| (ANC < | Resume brentuximab vedotin with pegfilgrastim or filgrastim |
| 1000/cumm) | (minimum of 7 days) at the previous dose in the subsequent cycle. |
| | If neutropenia recurs with use of growth factors, decrease |
| | brentuximab vedotin dose. Re-escalation is not allowed. |
| | If the patient is at the lowest allowed dose of brentuximab vedotin |
| | with pegfilgrastim or filgrastim, keep the dose of brentuximab |
| | vedotin the same and decrease the lenalidomide dose. |
| | If the patient is at the lowest allowed dose of both brentuximab |
| | vedotin and lenalidomide with pegfilgrastim or filgrastim, remove |
| | the patient from study. |
| Grade 3 or 4 | Hold brentuximab vedotin until toxicity resolves to ≤ grade 2. |
| thrombocytopenia | Resume brentuximab vedotin at the same dose level in the |
| (platelet count < | subsequent cycle. |
| 50,000/cumm)* | If grade 3 or 4 thrombocytopenia recurs with a lower dose of |
| | lenalidomide, then decrease brentuximab vedotin dose in the |
| | subsequent cycle. |
| | If the patient is at the lowest allowed dose of brentuximab vedotin, |
| | keep the dose of brentuximab vedotin the same and decrease the |
| | lenalidomide dose. |
| | If the patient is at the lowest allowed dose of both brentuximab |
| | vedotin and lenalidomide, remove the patient from study. |
| Grade 2, 3, or 4 | If grade 2, decrease brentuximab vedotin dose. |
| sensory neuropathy | If grade 3, hold brentuximab vedotin until toxicity resolves to ≤ |
| | grade 1. Decrease brentuximab vedotin dose. |
| | If grade 4, discontinue brentuximab vedotin. Remove patient |
| | from study. |
| Grade 2, 3, or 4 | If grade 2, decrease brentuximab vedotin dose. |
| motor neuropathy | If grade 3, discontinue brentuximab vedotin.. |
| | If grade 4, discontinue brentuximab vedotin. Remove patient |
| | from study. |
| Other grade 3 or 4 | Hold brentuximab vedotin until toxicity resolves to ≤ grade 2. |
| non-hematologic | For toxicity attributed to brentuximab vedotin, decrease |
| toxicity | brentuximab vedotin dose when restarting. If unrelated to |
| | brentuximab vedotin, resume at the previous dose level. |

There was no dose adjustments or modifications to brentuximab vedotin for infusion-related reactions. However, if anaphylaxis occured, brentuximab vedotin administration was immediately and permanently discontinued.

Lenalidomide dose could be delayed or modified according to the following tables:

| Starting | Reduction | Reduction | Reduction | Reduction | Reduction | Reduction |
|---|---|---|---|---|---|---|
| dose | 1 | 2 | 3 | 4 | 5 | 6 |
| 25 mg | 20 mg | 15 mg | 10 mg | 10 mg | 5 mg | Off study |
| | | | | Days 1-14 | | |
| 20 mg | 15 mg | 10 mg | 10 mg | 5 mg | Off study | Off study |
| | | | Days 1-14 | | | |
| 15 mg | 10 mg | 10 mg | 5 mg | Off study | Off study | Off study |
| | | Days 1-14 | | | | |

| NCI CTC | |
|---|---|
| Toxicity Grade | Dose Modification Instructions |
| Grade 3 or 4 | Hold lenalidomide until toxicity resolves to ≤ grade 2. |
| neutropenia | Follow CBC weekly. |
| (ANC < 1000/cumm) | Decrease lenalidomide 3 for the remainder of the current |
| | cycle. Do not make up missed doses. |
| | For subsequent cycles, use pegfilgrastim or filgrastim |
| | (minimum of 7 days). Resume lenalidomide at the starting |
| | dose from the previous cycle. |
| | Omitted doses are not made up. |
| | If neutropenia recurs with use of growth factors, decrease |
| | lenalidomide. Re-escalation is not allowed. |
| | If the patient is at the lowest allowed dose of brentuximab |
| | vedotin with pegfilgrastim or filgrastim, keep the dose of |
| | brentuximab vedotin the same and decrease the lenalidomide |
| | dose. |
| | If the patient is at the lowest allowed dose of both brentuximab |
| | vedotin and lenalidomide with pegfilgrastim or filgrastim, |
| | remove the patient from study. |
| Grade 3 or 4 | Hold lenalidomide. |
| thrombocytopenia | Follow CBC weekly. |
| (platelet count < | If thrombocytopenia resolves to ≤ grade 2 prior to Day 21 of |
| 50,000/cumm)* | the current cycle, decrease lenalidomide and continue through |
| | the scheduled Day 21 of the current cycle. Otherwise, omit |
| | for remainder of cycle. Decrease lenalidomide at the start of |
| | the next cycle. |
| | Omitted doses are not made up. |
| | If the patient is at the lowest allowed dose of brentuximab |
| | vedotin, keep the dose of brentuximab vedotin the same and |
| | decrease the lenalidomide dose. |
| | If the patient is at the lowest allowed dose of both brentuximab |
| | vedotin and lenalidomide, remove the patient from study. |
| Grade 3 or 4 non- | If grade 3, hold lenalidomide. Clinical team to follow at least |
| blistering rash | weekly. |
| | If the toxicity resolves to ≤ grade 1 prior to Day 21 of the current |
| | cycle, decrease lenalidomide and continue through the scheduled |
| | Day 21 of the current cycle. Otherwise, omit for remainder of |
| | cycle and decrease lenalidomide at the start of the next cycle. |
| | Omitted doses are not made up. |
| | If grade 4, discontinue lenalidomide. Remove patient from study. |
| Desquamating | Discontinue lenalidomide. Remove patient from study. |
| (blistering) rash - | |
| any grade | |
| Grade 3 or 4 sensory | If grade 3, hold lenalidomide until toxicity resolves to ≤ grade 2 |
| neuropathy | and decrease lenalidomide. |
| | If grade 4, discontinue lenalidomide. Remove patient from study. |
| Grade 3 or 4 motor | If grade 3, hold lenalidomide until toxicity resolves to ≤ grade 2 |
| neuropathy | and decrease lenalidomide. May continue single agent |
| | lenalidomide after discontinuing brentuximab vedotin if patient is |
| | deriving clinical benefit. |
| | If grade 4, discontinue lenalidomide. Remove patient from study. |
| Grade 3 or 4 venous | Hold lenalidomide and start therapeutic anticoagulation, if |
| thrombosis/embolism | appropriate. |
| | Restart lenalidomide at same dose or decrease at treating |
| | physician's discretion. |
| Other grade 3 or 4 | Hold lenalidomide until toxicity resolves to ≤ grade 2. |
| non-hematologic | If the toxicity resolves to ≤ grade 2 prior to Day 21 of the current |
| toxicity | cycle, resume lenalidomide and continue through the scheduled |
| | Day 21 of the current cycle. Otherwise, omit for remainder of |
| | cycle. |
| | Omitted doses are not made up. |
| | For toxicity attributed to lenalidomide, decrease. |

### Objectives

Primary Objectives

The primary objective was to determine the safety and maximum tolerated dose (MTh) of brentuximab vedotin in combination with lenalidomide in patients with rel/ref DLBCL.

Secondary Objectives
1. To test the potential association between CD30 expression on tumor cells and clinical efficacy of the combination of brentuximab vedotin and lenalidomide.
2. To evaluate the efficacy of BV and lenalidomide in the subsets of activated B cell-like and germinal center B cell-like DLBCL based on the Hans criteria.
3. To determine efficacy of brentuximab vedotin and lenalidomide in relapsed/refractory DLBCL as measured by the overall response rate (CR +PR), duration of response, and progression free survival (PFS).

Exploratory Objectives
1. To determine T-cell and NK cell subset numbers, phenotype, and functional status in rel/ref DLBCL patients, and whether the combination of brentuximab vedotin and lenalidomide alters these parameters during therapy.
2. To determine changes in plasma cytokine levels and other biomarkers in this patient population during therapy with the combination of brentuximab vedotin and lenalidomide.
3. To investigate for the presence of recurrent genomic mutations in pretreatment biopsies and correlate with therapy response.

### Determination of Antitumor Efficacy

The determination of antitumor efficacy was based on objective response assessments made according to the Revised Response Criteria for Malignant Lymphoma (Cheson, B.D., et al., J. Clin. Oncol. 25, 579-586 (2007)) and treatment decisions by the Investigator were based on these assessments. Clinical response of progressive disease (PD), stable disease (SD), partial response (PR), or complete response (CR) were determined at each assessment. Progressive disease included PD per Cheson 2007 and clinical disease progression per investigator.

A total of 37 subjects were treated with 1.2 mg/kg of BV on day 1 of each 21-day treatment cycle and 20 mg of lenalidomide on days 1-21 of each 21-day treatment cycle. Of these 37 subjects, 15 were CD30 positive and 22 were CD30 negative. Subjects were assessed with a median follow-up time of 14.3 months. Complete response (CR), partial response (PR), stable disease (SD), progressive disease (PD), and objective response rate (ORR) were determined. The results are summarized in the following table:

| | CD30 | CD30 | |
|---|---|---|---|
| | POSITIVE | NEGATIVE | |
| RESPONSE | (N = 15) | (N = 22) | OVERALL |
| CR | 6 (40%) | 7 (32%) | 13 (35%) |
| PR | 5 (33%) | 3 (14%) | 8 (22%) |
| SD | 1 (7%) | 4 (18%) | |
| PD | 3 (20%) | 8 (36%) | |
| ORR | 11 (73%) | 10 (46%) | 21 (57%) |
| (CR + PR) | | | |

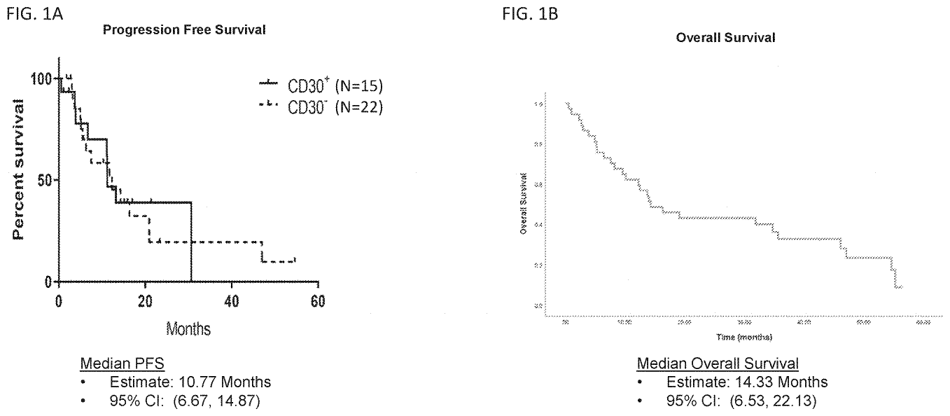

The progression free survival and overall survival were evaluated in the subjects. FIG. 1A shows the progression free survival of CD30+ and CD30− subjects and FIG. 1B shows the overall survival of subjects in the study.

### Example 2: A Randomized, Double-blind, Placebo-Controlled, Active-Comparator, Multicenter, Phase 3 Study of Brentuximab Vedotin or Placebo in Combination with Lenalidomide and Rituximab in Subjects with Relapsed or Refractory Diffuse Large B-Cell Lymphoma (DLBCL)

This is a randomized, double-blind, placebo-controlled, active-comparator, multicenter phase 3 study designed to evaluate the efficacy of brentuximab vedotin in combination with lenalidomide and rituximab versus placebo in combination with lenalidomide and rituximab for the treatment of subjects with relapsed or refractory (R/R) DLBCL. Approximately 400 subjects (approximately 200 subjects per arm) will be randomized in this study.

There will be a safety run-in period prior to the randomized portion of the study. Approximately 6 subjects will receive brentuximab vedotin 1.2 mg/kg intravenously, lenalidomide 20 mg orally, and rituximab 375 mg/m² intravenously. The safety and pharmacokinetic (PK) data from the first cycle of this run-in period will be evaluated prior to proceeding with the randomized portion of the study.

After completion of the safety run-in period, subjects will be randomized in a 1:1 manner to receive either brentuximab vedotin or placebo in combination with lenalidomide and rituximab and will be stratified by CD30 expression, prior allogenic or autologous stem cell transplant (SCT) therapy (received or not), prior CAR-T therapy (received or not), and cell of origin (germinal-center B-cell-like (GCB) or non-GCB). DLBCL and cell of origin (GCB or non-GCB) will be histologically determined by local pathology assessment. Subjects will have central pathology lab determination of CD30 expression by visual assessment of CD30 on tumor cells from a recent biopsy specimen by immunohistochemistry (IHC; using anti-CD30 BerH2 antibody) for stratification purposes. If, in the determination of the investigator, it is not medically feasible for the subject to undergo central pathology evaluation prior to randomization, and after discussion with the Medical Monitor, the subject may be stratified based on CD30 expression from the local pathology lab. Subjects who are stratified based on local pathology lab results must send in an archive block for central CD30 evaluation within 2 weeks of enrollment. To ensure sufficient power in the CD30-positive population, subjects with CD30-undetectable DLBCL will be capped at 50% of enrolled subjects. Subjects will be stratified based on a cut-off of >1% CD30 tumor expression; expression of >1% CD30 tumor expression will be considered CD30 positive while expression of <1% CD30 tumor expression will be considered CD30-undetectable.

### Study Population

Key eligibility criteria include subjects aged 12 and older with relapsed/refractory (R/R) DLBCL; subjects must have >2 prior lines of therapy and must be ineligible for stem cell transplant; subjects must have an Eastern Cooperative Oncology Group (ECOG) performance status score of 0 to 2; subjects must have a fluorodeoxyglucose (FDG)-avid disease by positron emission tomography (PET) and bidimensional measurable disease of at least 1.5 cm by computed tomography (CT), as assessed by the site radiologist.

### Investigation Product, Dose, and Mode of Administration

Brentuximab vedotin, 1.2 mg/kg via intravenous infusion every 3 weeks
Lenalidomide, 20 mg orally daily
Rituximab, 375 mg/m², via intravenous infusion on Cycle 1 Day 1
Rituximab 1400 mg via subcutaneous injection from Cycle 2 Day 1 through end of treatment.

### Control Product, Dose, and Mode of Administration

Placebo replacement for brentuximab vedotin will be administered via intravenous infusion every 3 weeks in the same manner as brentuximab vedotin
Lenalidomide, 20 mg orally daily
Rituximab, 375 mg/m², via intravenous infusion every 3 weeks
Rituximab 1400 mg via subcutaneous injection from Cycle 2 Day 1 through end of treatment.

### Duration of Treatment

Treatment may continue as long as there is clinical benefit (stable disease (SD) or better) without progression or unacceptable toxicity. The study ends once the number of events required for analysis of endpoints has been reached (estimated 5 years after first subject enrolled) or when the last subject completes the last visit, last contact, discontinues from the study, or is lost to follow-up, whichever occurs first.

### Efficacy Assessments

Subjects will be assigned a response status based on imaging and lymphoma assessments. Disease response will be assessed by a BICR and the investigator according to the Lugano Classification Revised Staging System for nodal non-Hodgkin and Hodgkin lymphomas. Radiographic disease evaluations, including contrast-enhanced CT scans of neck, chest, abdomen and pelvis, will be assessed at baseline and every 2 cycles thereafter. A PET scan is required at baseline and every 2 cycles thereafter. Once the PET is negative per the investigator, no further PET scans are required. A diagnostic quality CT-PET scan should also be performed at the time of suspected clinical progression.

### Pharmacokinetic and Immunogenicity Assessments

Blood samples will be obtained for PK and immunogenicity evaluation at protocol-defined time points. PK parameters to be estimated include maximum plasma concentration (Cₘₐₓ), the time Cₘₐₓ occurred (Tₘₐₓ), concentration at the end of infusion for brentuximab vedotin (Cₑₒᵢ), and trough concentration (C_{trough}). Immunogenicity will be evaluated with measurements of ADA in serum.

Blood will be collected for antidrug antibody (ADA) to brentuximab vedotin, brentuximab vedotin and monomethyl auristatin E (MMAE) exposures, and pharmacodynamic assessments

### Biomarker Assessments

Tumor samples will be collected for assessment of CD30 antigen expression, mRNA levels of CD30 and related genes, and cell of origin classification. Blood will be collected for soluble CD30 and other chemokines/cytokines of interest.

### Safety Assessments

Safety assessments will include the surveillance and recording of adverse events (AEs), physical examination findings, and laboratory tests.

### Other Assessments

Patient-Reported Outcomes and Health Economics: Health outcomes assessments will include health-related quality of life and healthcare utilization, which will be described in the statistical analysis plan (SAP).

### Statistical Methods

### Stratification:

Subjects will be stratified by CD30 status (positive or undetectable based on a cut-off of ≥1% CD30 tumor expression) by central pathology review, cell of origin (GCB or non-GCB), prior treatment with CAR-T (received or not), and prior SCT therapy (received or not).

### Sample Size Considerations:

In order to evaluate the dual primary endpoint of PFS in the intent-to-treat (ITT) population and in CD30-positive subjects, an exhaustive fallback testing approach will be used to control overall type I error rate. PFS in the ITT population will be tested at a 2-sided alpha of 0.03 and PFS in CD30 positive subjects will be tested at a 2-sided alpha of 0.02. If 1 of the 2 endpoints meets statistical significance, the other can be tested again at an alpha level of 0.05.

Under this proposed testing strategy, assuming a hazard ratio (HR) of 0.62 for both the ITT population and the CD30-positive population, approximately 280 PFS events are required to achieve at least 90% power in ITT population. Under these assumptions approximately 140 events are expected to be observed for the CD30-positive group, which will provide at least 80% power. Calculations are based on a 2-sided alpha level of 0.05 using the log-rank test.

The accrual period is expected to be approximately 24 months, with additional follow up to reach the specified number of events. The PFS rates in the control arm are based on Czuczman M S, et al. Clin. Cancer Res. 2017 Aug. 1; 23(15):4127-37. doi: 10.1158/1078-0432.CCR-16-2818 (i.e., 50% at 3 months, 20% at 6 months, and 18% at 12 months). Assuming a HR of 0.62, and a 5% annual dropout rate, approximately 400 subjects will be randomized.

An interim analysis of ORR is planned after completion of enrollment. The analysis will include a minimum of 250 patients (125 per arm) who have completed 6 months of follow-up. Assuming an ORR of 57% in the experimental arm and an ORR of 28% in the control arm, this will provide at least 90% power to detect a difference in ORR between the 2 arms, based on Fisher's exact test at a 2-sided alpha of 0.005.

The key secondary endpoint of OS will be tested in the ITT population and the CD30-positive population at 2 time points. An interim analysis will be conducted at the time of the PFS analysis and a final analysis will be conducted after 300 OS events have been observed. Power for the ITT population and CD30-positive group are similar to those for the PFS analysis described above.

### Analysis Methods:

For the primary efficacy analyses of PFS per blinded independent central review (BICR) in the ITT population and in CD30-positive subjects, the stratified log-rank test will be used to compare PFS between the 2 treatment groups. The HR will be estimated using a stratified Cox regression model. PFS will also be summarized using the Kaplan-Meier method. Similar methods will be used for the secondary efficacy endpoint of OS, and other time-to-event efficacy endpoints.

### Analysis Timing

The estimated duration of the study through final primary analysis is approximately 2.5 years from the randomization of the first subject (including approximately 2 years of enrollment and an additional 6 months of follow-up to reach the specified number of PFS events). The interim analysis of ORR will occur after the completion of enrollment (2 years). The final analysis of OS is expected to occur approximately 1 year from the final primary analysis (3.5 years).

### Objectives

This study will evaluate the efficacy of brentuximab vedotin in combination with lenalidomide and rituximab among subjects with relapsed or refractory CD30-positive (CD30 expression≥1%) or CD30-undetectable (CD30 expression<1%) DLBCL. Specific objectives and corresponding endpoints for the study are summarized below.

Study Objectives and Endpoints

| Primary Objectives | Corresponding Dual Primary Endpoints |
|---|---|
| Evaluate and compare progression-free | PFS^{a} per blinded independent central review |
| survival (PFS) between the 2 treatment | (BICR) in the ITT population |
| arms in the intent-to-treat (ITT) | PFS per BICR in the CD30(+) population. |
| population | |
| Evaluate and compare PFS between the | |
| 2 treatment | |
| arms in the CD30(+) population | |
| Key Secondary Objectives | Corresponding Key Secondary Endpoints |
| Evaluate and compare the objective | ORR per BICR |
| response rate (ORR) between the 2 | |
| treatment arms in the ITT | |
| population | |
| Evaluate and compare OS between the 2 | OS in the ITT population |
| treatment arms in the ITT population | OS in the CD30(+) population |
| Evaluate and compare overall survival | |
| (OS) between | |
| the 2 treatment arms in the CD30(+) | |
| population | |
| Other Secondary Objectives | Corresponding Other Secondary Endpoints |
| Evaluate and compare the complete | CR rate |
| response (CR) rate between the 2 | |
| treatment arms | |
| Evaluate and compare duration of | Duration of objective response |
| response between the 2 treatment arms | |
| Evaluate the safety and tolerability | Incidence, severity, and seriousness of adverse |
| of the 2 treatment arms | events (AEs) per National Cancer Institute Common |
| | Terminology Criteria for Adverse Events |
| | (NCI CTCAE) version 5.0 |
| Exploratory Objectives | Corresponding Exploratory Endpoints |
| To characterize the relationship of | Association of CD30 expression with ORR and |
| CD30 expression with clinical | PFS |
| responses to brentuximab vedotin, | |
| lenalidomide, and rituximab | |
| To evaluate the utility of RECIL 2017 | ORR and PFS based on RECIL 2017 response |
| response assessments in DLBCL | criteria |
| To assess the serum pharmacokinetic | Brentuximab vedotin serum concentrations and |
| (PK) and immunogenicity of | incidence of antidrug antibody (ADA) to |
| brentuximab vedotin | brentuximab vedotin |
| To explore the relationship between | Association of molecular biomarkers with ORR |
| prognostic molecular phenotype markers | and PFS. |
| and clinical responses to brentuximab | |
| vedotin, lenalidomide, and rituximab | |
| To explore the relationship of | EuroQol-5 dimension-5 level (EQ-5D-5L), |
| responses to patient-reported | National Comprehensive Cancer Network/Functional |
| outcomes (PROs) and healthcare | Assessment of Cancer Therapy-Lymphoma |
| utilization between the 2 | (NCCN FACT-Lym), and healthcare utilization |
| treatment arms | |

^{a}The dual-primary efficacy endpoints of this study are PFS by BICR in the ITT population and in the CD30(+) population. PFS is defined as the time from the date of randomization to the date of first documentation of progressive disease (PD), or death due to any cause, whichever occurs first.

### Study Population

Subjects must meet all of the enrollment criteria to be eligible for this study.

Inclusion Criteria
1. Subjects with relapsed or refractory diffuse and transformed large B-cell lymphoma (R/R DLBCL). DLBCL will be histologically determined by local pathology assessment for the purposes of study eligibility.
2. Subjects must have R/R disease following ≥2 lines of prior systemic therapy.
3. Subjects must be ASCT or CAR-T ineligible according to the investigator and must meet at least one of the following criteria:
   a. One or more co-morbidities, including cardiac, pulmonary, renal or hepatic dysfunction
   b. Active disease following induction and salvage chemotherapy
   c. Inadequate stem cell mobilization (for ASCT)
   d. Relapse following prior ASCT or CAR-T
   e. Unable to receive CAR-T therapy due to financial, geographic, or insurance issues.
4. Subjects will need to have a formalin-fixed paraffin-embedded tumor block (obtained ≤4 weeks before Day 1) submitted to the central pathology lab for the determination of CD30 expression, which will be centrally determined by visual assessment for any detectable level of CD30 on tumor cells by IHC (using the anti-CD30 BerH2 antibody). If a recent biopsy is not available (obtained ≤4 weeks before Day 1) and a biopsy is not medically feasible or appropriate, contact the medical monitor. If, in the determination of the investigator, it is not feasible for the subject to undergo central pathology evaluation prior to randomization, and after discussion with the Medical Monitor, the subject may be stratified based on CD30 expression from the local pathology lab. Subjects who are stratified based on local pathology lab results must have an archive block sent in for central CD30 evaluation within 2 weeks of enrollment.
5. Age 12 and older.
6. An Eastern Cooperative Oncology Group (ECOG) performance status score of 0 to 2.
7. Subjects must have fluorodeoxyglucose (FDG)-avid disease by positron emission tomography (PET) and bidimensional measurable disease of at least 1.5 cm by computed tomography (CT), as assessed by the site radiologist within 28 days of Day 1.
8. The following baseline laboratory data within 28 days of Day 1:
   a. Absolute neutrophil count (ANC)≥1000/μL.
   b. Platelet count >50,000/μL with no platelet transfusion or growth factor support in the 28 days prior to Day 1.
   c. Serum bilirubin ≤1.5× upper limit of normal (ULN) or ≤3×ULN for subjects with Gilbert's disease or documented hepatic involvement with lymphoma.
   d. Estimated glomerular filtration rate (GFR)≥60 mL/min/1.73 m² using the

Modification of Diet in Renal Disease (MDRD) study equation.

GFR (mL/min/1.73 m²)=175×(Scr)^{-1.154}×(Age)^{-0.203}×(0.742 if female)×(1.212 if African American)
e. Alanine aminotransferase (ALT) and aspartate aminotransferase (AST) ≤3.0×ULN or 5.0×ULN for subjects with documented hepatic involvement with lymphoma.
9. Subjects of childbearing potential must avoid pregnancy for at least 4 weeks before beginning lenalidomide therapy, during therapy, during dose interruptions, and for at least 6 months after completing therapy. Subjects must commit either to abstain continuously from heterosexual sexual intercourse or to use 2 methods of reliable birth control, beginning 4 weeks prior to initiating treatment with lenalidomide, during therapy, during dose interruptions, and continuing for 6 months following discontinuation of lenalidomide therapy. Two negative serum beta human chorionic gonadotropin (β-hCG) pregnancy tests must be obtained prior to initiating therapy. The first test should be performed within 10 to 14 days and the second test performed within 48 hours prior to receiving lenalidomide therapy. Afterwards, a serum β-hCG pregnancy test must be administered weekly during the first month, then monthly thereafter in females with regular menstrual cycles or every 2 weeks in subjects with irregular menstrual cycles.
10. If sexually active in a way that could result in pregnancy, subjects of childbearing potential and subjects who can father children and have partners of childbearing potential must agree to use 2 effective contraception methods during the study and for 6 months following the last dose of study drug.
11. The following requirements for subjects who are human immunodeficiency virus (HIV)-positive:
   CD4+ T-cell counts ≥350 cell/μL within 28 days of Day 1
   No acquired immune deficiency syndrome-defining opportunistic infection within the past 12 months
   On established highly active antiretroviral therapy for at least 4 weeks with an HIV viral load less than 400 copies/mL within 28 days of Day 1
12. Subjects must be registered into the mandatory lenalidomide REMS® program and be willing to comply with its requirements. Per standard lenalidomide REMS® program requirements, all physicians who prescribe lenalidomide for research subjects enrolled into this trial, must be registered in, and must comply with, all requirements of the lenalidomide REMS® program.
13. The subject or the subject's legally acceptable representative must provide written informed consent. For subjects less than 18 years old, a parent or legally acceptable representative must provide written informed consent; if applicable, the subject should also provide assent per institutional standards.

Exclusion Criteria
1. History of another malignancy within 2 years before the first dose of study drug or any evidence of residual disease from a previously diagnosed malignancy. Exceptions are malignancies with a negligible risk of metastasis or death (e.g., 5-year OS≥90%), such as carcinoma in situ of the cervix, non-melanoma skin carcinoma, localized prostate cancer, ductal carcinoma in situ, or Stage I uterine cancer.
2. History of progressive multifocal leukoencephalopathy (PML).
3. Active cerebral/meningeal disease related to the underlying malignancy. Subjects with a history of cerebral/meningeal disease related to the underlying malignancy are allowed if prior CNS disease has been effectively treated and without progression for at least 3 months.
4. Any uncontrolled Grade 3 or higher (per NCI CTCAE version 5.0) viral, bacterial, or fungal infection within 2 weeks prior to the first dose of study drug. Routine antimicrobial prophylaxis is permitted.
5. Chemotherapy, radiotherapy, biologics, and/or other antitumor treatment with immunotherapy that is not completed 3 weeks prior to first dose of study drug, unless underlying disease has progressed on treatment.
6. Subjects who are breastfeeding.
7. Known hypersensitivity to any study drug or excipient contained in the drug formulation of the study drugs.
8. Known to be positive for hepatitis B by surface antigen expression. Known to be positive for hepatitis C infection (positive by polymerase chain reaction [PCR] or on antiviral therapy for hepatitis C within the last 6 months). Subjects who have been treated for hepatitis C infection are permitted if they have documented sustained virologic response of 12 weeks.
9. Subjects with previous allogeneic SCT if they meet either of the following criteria:
   <100 days from allogeneic SCT
   Active acute or chronic graft-versus-host disease (GVHD) or receiving immunosuppressive therapy as treatment for or prophylaxis against GVHD.
10. Previous treatment with brentuximab vedotin or lenalidomide.
11. Current therapy with immunosuppressive medications (including steroids), other systemic anti-neoplastic, or investigational agents.
12. Documented history of a cerebral vascular event (stroke or transient ischemic attack), unstable angina, myocardial infarction, or cardiac symptoms consistent with New York Heart Association (NYHA) Class III-IV within 6 months prior to the first dose of study drugs.
13. Congestive heart failure, Class III or IV, by the NYHA criteria.
14. Grade 2 or higher peripheral sensory or motor neuropathy at baseline.
15. Other serious underlying medical condition that, in the opinion of the investigator, would impair the subject's ability to receive or tolerate the planned treatment and follow-up.

### Treatments

### Treatments Administered

The treatment regimens consist of either brentuximab vedotin or placebo in combination with lenalidomide and rituximab. Brentuximab vedotin, the investigational agent under study in this protocol, is an ADC consisting of the antibody cAC10, specific for human CD30; the microtubule-disrupting agent MMAE; and a protease-cleavable linker that covalently attaches MMAE to cAC10. Lenalidomide is an immunomodulatory drug. Rituximab is a chimeric monoclonal antibody that recognizes CD20, a cell surface antigen typically found on B-lymphocytes and most B-cell lymphomas.

In the setting of the specific toxicity for a single component, the other agents may be continued.

For the control arm (placebo in combination with lenalidomide and rituximab), the site pharmacist will prepare a placebo replacement for brentuximab vedotin (e.g., normal saline) to be administered in the same manner as brentuximab vedotin.

Brentuximab vedotin is a sterile, preservative free, white to off-white lyophilized cake or powder supplied by Seattle Genetics in single-use vials for reconstitution for IV administration. Each vial of the product contains brentuximab vedotin, trehalose, sodium citrate, and polysorbate 80.

Brentuximab vedotin 1.2 mg/kg will be administered on Day 1 of each 21-day cycle by intravenous (IV) infusion given over approximately 30 minutes to patients randomized to treatment with brentuximab in combination with lenalidomide and rituximab. In the absence of infusion-related reactions, the infusion rate for all subjects should be calculated in order to achieve a 30-minute infusion period. Brentuximab vedotin must not be administered as an IV push or bolus. Brentuximab vedotin should not be mixed with other medications.

Weight-based dosing is based on subject actual body weight. Doses must be adjusted for subjects who experience a ≥10% change in weight from baseline. Subject weight must be measured during all relevant assessment windows as described in the schedule of events. Other dose adjustments for changes in body weight are permitted per institutional standard. Rounding is permissible within 5% of the nominal dose. An exception to weight-based dosing is made for subjects weighing greater than 100 kg; doses will be capped at 100 kg for these individuals. The maximum dose calculated per cycle in this study is 120 mg for subjects receiving the 1.2 mg/kg dose level.

Doses reduced for treatment-related toxicity should not be re-escalated without discussion with the sponsor. In the event of dose modification or discontinuation of brentuximab vedotin, the other study agents may continue to be administered. Brentuximab vedotin dosing may continue to be administered if the either or both of the other study agents are discontinued.

The following table describes the recommended dose modifications for study treatment-associated toxicity.

Recommended dose modifications for brentuximab vedotin-associated toxicity

| Toxicity | Grade 1 | Grade 2 | Grade 3 | Grade 4 |
|---|---|---|---|---|
| Sensory | Continue | Continue | Withhold BV/placebo | Discontinue treatment |
| Neuropathy | BV/placebo at | BV/placebo at | until PN is ≤ Grade 2, | with BV/placebo |
| | same dose level | same dose level | then reduce dose to | |
| | | | 0.9 mg/kg and resume | |
| | | | treatment; if already at | |
| | | | 0.9 mg/kg, continue | |
| | | | dosing at that dose. | |
| Motor Neuropathy | Continue | Reduce | Discontinue treatment | Discontinue treatment |
| | BV/placebo at | BV/placebo dose | with BV/placebo | with BV/placebo |
| | same dose level | to 0.9 mg/kg and | | |
| | | resume treatment | | |
| Non-hematologic | Continue | Continue | Withhold BV/placebo | Withhold BV/placebo |
| (except peripheral | BV/placebo at | BV/placebo at | until toxicity is ≤ | until toxicity is ≤ |
| neuropathy) | same dose level | same dose level | Grade 2 or has | Grade 2 or has |
| | | | returned to baseline, | returned to baseline, |
| | | | then resume treatment at | then reduce dose to |
| | | | the same dose level^{b}. | 0.9 mg/kg and resume |
| | | | | treatment, or |
| | | | | discontinue at the |
| | | | | discretion of the |
| | | | | investigator^{a,b,c}. |

| | | | |
|---|---|---|---|
| Hematologic^{d} | Continue | Continue | Hold BV/placebo dose until event resolves |
| | BV/placebo and | BV/placebo and | to ≤ Grade 2 or baseline. Follow |
| | lenalidomide at | lenalidomide at | lenalidomide dose modifications as noted |
| | same dose level | same dose level | |

^{a}Dose reduction below 0.9 mg/kg are not allowed, and toxicities should be managed with dose delays.
^{b}Subjects who develop Grade 3 or 4 electrolyte laboratory abnormalities may continue study treatment without interruption.
^{c}Treatment should be discontinued for subjects who experience Grade 4 infusion-related reactions.
^{d}Support with blood product transfusions allowed per institutional standard of care.

Refrigeration should be set at 2 to 8° C. for storage of vials and solutions containing brentuximab vedotin. Brentuximab vedotin does not contain preservatives; therefore, opened and reconstituted vials of brentuximab vedotin should be used as soon as possible. It is recommended that brentuximab vedotin vials and solutions be protected from direct sunlight until the time of use. Reconstituted vials and solutions must not be shaken.

Brentuximab vedotin vials are provided via single-use containers. Any partially used vials or diluted dosing solutions should be discarded using appropriate institutional drug disposal procedures.

Brentuximab vedotin should be reconstituted with the appropriate amount of Sterile Water for Injection, United States Pharmacopeia or equivalent. The vial should be gently swirled until the contents are completely dissolved. The vial must not be shaken. The reconstituted drug product should be inspected visually for any particulate matter and discoloration.

The required volume of reconstituted drug product should be diluted into an infusion bag. The bag should be gently inverted to mix the solution. The bag must not be shaken. Prior to administration, the reconstituted and diluted drug product should be inspected visually for any particulate matter and discoloration.

All subjects in this study will receive lenalidomide. Lenalidomide is an immunomodulatory drug. Lenalidomide will be provided as 5 mg and 10 mg capsules.

Lenalidomide is commercially available and approved by the US Food and Drug Administration (FDA) to treat subjects with relapsed or refractory MCL, MM, and MDS.

Lenalidomide 20 mg will be administered orally once daily.

Lenalidomide should be administered with water. The capsule should be swallowed intact and subjects should not attempt to chew capsules, open capsules or dissolve them in water. Each dose of lenalidomide should be taken with or without food, at approximately the same time each day.

Dose modification guidelines as summarized in this section are recommended to manage Grade 3 or 4 thrombocytopenia or neutropenia or other Grade 3 or 4 toxicities considered to be related to lenalidomide. In the event of dose modification or discontinuation of lenalidomide, the other study agents may continue to be administered. Lenalidomide may continue to be administered if brentuximab vedotin or rituximab dosing is permanently discontinued due to toxicity.

Platelet Counts- Thrombocytopenia during treatment

| When Platelets | Recommended Course |
|---|---|
| Fall to <50,000 mcL | Interrupt lenalidomide treatment |
| | and follow CBC weekly |
| Return to ≥50,000/mcL | Resume lenalidomide at 5 mg less |
| | than the previous dose. Do not |
| | dose below 5 mg daily |

Absolute Neutrophil Counts (ANC)- Neutropenia during treatment

| When Neutrophils | Recommended Course |
|---|---|
| Fall to <1000/mcL for | Interrupt lenalidomide treatment |
| at least 7 days OR | and follow CBC weekly |
| Fall to <1000/mcL with an | |
| associated temperature ≥38.5° C. | |
| OR Fall to <500/mcL | |
| Return to ≥1000/mcL | Resume lenalidomide at 5 mg less |
| | than the previous dose. Do not |
| | dose below 5 mg daily |

For other Grade 3 or 4 toxicities considered to be related to lenalidomide, hold treatment and restart at the physician's discretion at next lower dose level when toxicity has resolved to ≤Grade 2.

All subjects in this study will receive rituximab. Rituximab is a chimeric monoclonal antibody that recognizes CD20, a cell surface antigen typically found on B-lymphocytes and most B-cell lymphomas.

Rituximab is commercially available and approved by the US Food and Drug Administration (FDA) to treat subjects with relapsed or refractory non-Hodgkin's lymphoma and chronic lymphocytic leukemia.

Rituximab 375 mg/m² will be administered after administration of brentuximab vedotin on Cycle 1 Day 1 (±1 day) via intravenous infusion according to the package insert or institutional standard of care. The rituximab infusion will begin within approximately 60 to 90 minutes after the end of the brentuximab vedotin infusion.

Beginning from Cycle 2 Day 1, all subjects will receive rituximab 1400 mg via subcutaneous injection.

Additional premedications, including steroids, may be given prior to the rituximab infusion in accordance with the rituximab package insert, institutional standard of care, or as clinically indicated.

Rituximab may continue to be administered if brentuximab vedotin or lenalidomide dosing is permanently discontinued due to toxicity. No dose reductions of rituximab are allowed; however, if unacceptable toxicity to rituximab develops, rituximab may be permanently discontinued and brentuximab vedotin and lenalidomide dosing may continue. Rituximab should be permanently discontinued in HIV-positive patients who have CD4+counts <100 cells/mm³.

### Required Premedication and Postmedication

Routine premedication should not be administered for the prevention of infusion-related reactions prior to the first dose of brentuximab vedotin. However, subjects who experience a Grade 1 or Grade 2 infusion-related reaction may receive subsequent brentuximab vedotin infusions with premedication. Subjects who experience a Grade 3 or Grade 4 infusion-related reaction may potentially receive additional treatment with brentuximab vedotin at the discretion of the investigator after discussion with the sponsor.

Acetaminophen and an antihistamine should be given within 30 to 60 minutes prior to initiating the rituximab intravenous infusion on Cycle 1 Day 1. Additional premedications, including steroids, may be given prior to the rituximab infusion in accordance with the rituximab package insert, institutional standard of care, or as clinically indicated.

Subjects should be individually evaluated to assess the need for tumor lysis prophylaxis prior to the first dose of brentuximab vedotin. Subjects should receive prophylaxis as appropriate per the institutional standards.

### Concomitant Therapy

All patients must take low-dose aspirin (81 mg daily) as prophylactic anticoagulation, unless already receiving anticoagulation therapy. Patients intolerant to ASA may use warfarin or low molecular weight heparin.

Medications for infusion-related reactions during brentuximab vedotin and rituximab administration, such as epinephrine, antihistamines, and corticosteroids, should be available for immediate use.

Patients who experience a Grade 1 or Grade 2 infusion-related reaction may receive subsequent brentuximab vedotin/placebo infusions with premedication consisting of acetaminophen and diphenhydramine administered 30 to 60 minutes prior to each 30-minute infusion or according to institutional standards. If anaphylaxis occurs, brentuximab vedotin/placebo administration should be immediately and permanently discontinued; lenalidomide and rituximab administration may continue.

The use of platelet and/or red blood cell supportive growth factors or transfusions when applicable is allowed. The use of colony stimulating factors for the treatment of neutropenia per institutional practice is permitted during therapy. Prednisone (or equivalent) ≤10 mg/day may be used for non-lymphomatous purposes.

Primary or secondary granulocyte-colony stimulating factor (G-CSF) prophylaxis is strongly recommended. When G-CSF prophylaxis is used, it should be administered 1 to 3 days after brentuximab vedotin/placebo administration. The type of G-CSF formulation used may be per institutional guidelines.

Subjects who are receiving strong CYP3A4 inhibitors concomitantly with brentuximab vedotin/placebo should be closely monitored for adverse reactions. Examples of strong CYP3A4 inhibitors include clarithromycin, telithromycin, nefazodone, itraconazole, ketoconazole, atazanavir, darunavir, indinavir, lopinavir, nelfinavir, ritonavir, saquinavir, and tipranavir.

### Schedule Of Events

Adverse events and concomitant medications will be recorded from Day 1 (predose) through the safety reporting period. Any study protocol-related AE, as well as any concomitant medications given for treatment of the AE, should be recorded from the time of informed consent.

Clinical laboratory assessments (serum chemistry panel, complete blood count [CBC] with differential [manual differential if clinically indicated], physical exam, weight, and performance status) may be performed within 1 day prior to administration of study drug. The results from all relevant clinical laboratory assessments must be reviewed prior to dosing.

A schedule of events is provided in the following table.

| | | | | | Day of 21-Day | | | | Long-term F/U | | | | | | |
|---|---|---|---|---|---|---|---|---|---|---|---|---|---|---|---|
| | | Baseline/ | | | Combination | | | EOT | Months after first dose | | | | | | |
| | | Screening | | Enrollment | Treatment Cycle | | | visit | | | | | | | >24 |
| | | D | D | D | | | Between | 30-37 days | | | | | | | Every 6 |
| | | −28 | −7 | −7 | D | D 2 | D 15 | post last | 9 | 12 | 15 | 18 | 21 | 24 | months ± |
| Day | | to 1 | to 1 | to 1 | 1 | to 21 | to 21 | dose^{d} | ±1 week | | | | | | 1 week |
| Visit Window | | | | | ±2 d | | | | | | | | | | |
| Screening/ | Informed | X | | Eligibility | | | | | | | | | | | |
| Baseline | consent | | | documentation | | | | | | | | | | | |
| Assessments | Inclusion/ | X | | submitted | | | | | | | | | | | |
| | exclusion | | | to sponsor | | | | | | | | | | | |
| | Medical | X | | prior to | | | | | | | | | | | |
| | history | | | study start; | | | | | | | | | | | |
| | Tumor Biopsy for | X | | randomization^{e} | | | | | | | | | | | |
| | Central lab^{a} | | | | | | | | | | | | | | |
| | Hgb A1c or | X | | | | | | | | | | | | | |
| | fasting | | | | | | | | | | | | | | |
| | glucose | | | | | | | | | | | | | | |
| | Height | | X | | | | | | | | | | | | |
| | Weight | | X | | X | | | | | | | | | | |
| | Electrocar- | | X | | | | | | | | | | | | |
| | diogram | | | | | | | | | | | | | | |
| | Vital signs | | X | | X | | | | | | | | | | |
| | Serology | X | | | | | | | | | | | | | |
| | (hepatitis | | | | | | | | | | | | | | |
| | B and C, EBV) | | | | | | | | | | | | | | |
| | Pregnancy test by | | X | | X | X | X | X | | | | | | | |
| | serum β-hCG^{b} | | | | | | | | | | | | | | |
| | ECOG performance | | X | | X | | | X | | | | | | | |
| | status | | | | | | | | | | | | | | |
| | Registration into | X | | | | | | | | | | | | | |
| | lenalidomide | | | | | | | | | | | | | | |
| | REMS program | | | | | | | | | | | | | | |
| Treatment | Study treatment | | | | X | | | | | | | | | | |
| | administration - | | | | | | | | | | | | | | |
| | brentuximab | | | | | | | | | | | | | | |
| | vedotin or placebo | | | | | | | | | | | | | | |
| | Study treatment | | | | X | X | | | | | | | | | |
| | administration - | | | | | | | | | | | | | | |
| | lenalidomide | | | | | | | | | | | | | | |
| | Study treatment | | | | X | | | | | | | | | | |
| | administration - | | | | | | | | | | | | | | |
| | rituximab | | | | | | | | | | | | | | |
| ADA/PK/ | Samples for | | | | | | | | | | | | | | |
| Pharmaco- | ADA/PK/ | | | | | | | | | | | | | | |
| dynamics | Pharmaco- | | | | | | | | | | | | | | |
| | dynamics | | | | | | | | | | | | | | |
| Response | CT^{c} (neck, chest, | X | | | | | | | | | | | | | |
| Assessment | abdomen, pelvis) | X | | | | | | | | | | | | | |
| | PET^{c} (Whole | | | | | | | | | | | | | | |
| | body) Lugano | | | | | | | | | | | | | | |
| | 2014 Response | | | | | | | | | | | | | | |
| | Assessment | | | | | | | | | | | | | | |
| | Survival status | | | | | | | | | X^{d} | | X^{d} | | X^{d} | X^{e} |
| | and subsequent | | | | | | | | | | | | | | |
| | treatment | | | | | | | | | | | | | | |
| Safety | Serum chemistry | | X | | X | | | X | | | | | | | |
| Assessments | CBC with | | X | | X | | | X | | | | | | | |
| | differential | | | | | | | | | | | | | | |
| | Creatinine | | X | | X | | | X | | | | | | | |
| | clearance | | | | | | | | | | | | | | |
| | Concomitant | Collect any related | | | Collect from Day 1 (predose) | | | | | | | | | | |
| | meds and AEs | to study protocol | | | through 30 days post last | | | | | | | | | | |
| | | procedures | | | dose or through EOT visit, | | | | | | | | | | |
| | | | | | whichever is later | | | | | | | | | | |
| PRO/MRU | Healthcare | | | | X | | | X | | | | | | | |
| | Utilization | | | | | | | | | | | | | | |
| | NCCN | | | | X | | | X | | | | | | | |
| | FACT-Lym | | | | | | | | | | | | | | |
| | EQ-5D-5L | | | | X | | | X | | | | | | | |

^{a}Tumor specimen must be submitted for central pathology review before randomization to confirm CD30 expression. All subjects will have central pathology lab determination of CD30 expression by visual assessment of CD30 on tumor cells from a recent biopsy specimen by immunohistochemistry (IHC; using anti-CD30 BerH2 antibody) for stratification purposes. If, in the determination of the investigator, it is not feasible for the subject to undergo central pathology evaluation prior to randomization, and after discussion with the Medical Monitor, the subject may be stratified based on CD30 expression from the local pathology lab.
^{b}Subjects of childbearing potential must avoid pregnancy for at least 4 weeks before beginning lenalidomide therapy, during therapy, during dose interruptions, and for at least 6 months after completing therapy. Subjects must commit either to abstain continuously from heterosexual sexual intercourse or to use 2 methods of reliable birth control, beginning 4 weeks prior to initiating treatment with lenalidomide, during therapy, during dose interruptions, and continuing for 6 months following discontinuation of lenalidomide therapy. Two negative serum B-HCG pregnancy tests must be obtained prior to initiating therapy. The first test should be performed within 10 to 14 days and the second test within 48 hours prior to receiving lenalidomide therapy. Afterwards, a serum B-HCG pregnancy test must be administered weekly during the first month, then monthly thereafter in females with regular menstrual cycles or every 2 weeks in subjects with irregular menstrual cycles. If sexually active in a way that could result in pregnancy, subjects of childbearing potential and subjects who can father children and have partners of childbearing potential must agree to use 2 effective contraception methods during the study and for 6 months following the last dose of study drug.
^{c}A combined CT/PET may be obtained to satisfy the requirements for CT and PET scanning, as long as a diagnostic quality CT scan is obtained; PET scans may also be obtained any time during the study if clinically indicated. Diagnostic-quality contrast-enhanced CT scans of neck, chest, abdomen and pelvis, will be assessed at baseline and every 6 weeks thereafter. A diagnostic quality CT-PET scan should also be performed at the time of suspected clinical progression. A PET scan is required at baseline and every 6 weeks thereafter. Once the PET is negative per the investigator, no further PET scans are required.
^{d}Once a patient experiences PD per investigator assessment, survival status is required every 6 months until death or study closure, whichever comes first. After 30 months, the window for the assessment is ±1 month. Collect information regarding subsequent anticancer therapies.
^{e}Randomization to occur after eligibility is determined and within 1 business day of planned first dose of study treatment.

### Response/Efficacy Assessments

The determination of antitumor activity will be based on response assessments made according to the Lugano Classification Revised Staging System for nodal non-Hodgkin and Hodgkin lymphomas (Cheson BD, et al. J Clin Oncol 32(27): 3059-68 (2014)). Staging will be performed by PET/CT of diagnostic quality, with disease involvement determined by focal FDG uptake in nodal and extranodal (including spleen, liver, bone marrow, and thyroid) sites that is consistent with lymphoma, according to the pattern of uptake and/or CT characteristics. Up to 6 of the largest nodes, nodal masses, or other involved lesions that are measurable in 2 diameters should be identified as target lesions; if possible, they should be from disparate regions of the body and they should include mediastinal and retroperitoneal areas of disease whenever these sites are involved.

Both PET and CT scanning will be required until site radiologist determines disease is PET negative; responses will then be followed by CT scan of diagnostic quality only. Progressive metabolic disease (PmD), no metabolic response, partial metabolic response, or complete metabolic response will be determined using PET-based response at each assessment after baseline. The PET scan metabolic uptake will be graded using the Deauville 5-point scale with a score of <3 considered to represent a complete metabolic response. In addition to assessing metabolic response by Deauville, an evaluation of new lesions and bone marrow uptake will be included when evaluating PET/CT-based response. (Cheson 2014). If only CT-based assessment is performed, response will be categorized as PD, stable disease (SD), partial remission (PR), or CR. Evaluation of non-measured lesions, organ enlargement and new lesions will be included in the CT-based response assessment (Cheson 2014). PmD/PD includes radiological evidence of progression per Lugano classification criteria. If clinical progression is determined by the investigator, radiographic staging should also be performed to determine response assessment per Lugano classification criteria.

At any timepoint, if the PET scan and/or the CT scan show evidence of progressive disease, but there is no evidence of clinical progression, the investigator is permitted to continue the investigational therapy until PET and CT scans are repeated 4+weeks later (confirmatory scans). If the confirmatory scans confirm progression, then treatment should be discontinued, and the date of progression should be the date of the initial scans that documented PD. However, if progression is not seen on the confirmatory scans, then treatment may be continued, and subsequent imaging and other study activities should be conducted per the Schedule of Events.

At any time on study treatment, if there is no evidence of clinical progression, and if it is felt to be in the best interest for the subject, treatment may continue beyond PD. Follow up PET-CT imaging studies should be obtained in such cases, until treatment is discontinued.

### Data Analysis Methods

In order to evaluate the dual primary endpoint of PFS in the ITT population and in the CD30-positive population, an exhaustive fallback testing approach will be used to control overall type I error rate at a 2-sided alpha level of 0.05 (assigning α₁=0.6*alpha to PFS in ITT, and α₂=0.4*alpha to PFS in CD30 positive).

Under the proposed testing strategy, approximately 280 PFS events are required for the primary analysis to detect a hazard ratio (HR) of 0.62 using the log-rank test. Under these assumptions approximately 140 events are expected to be observed for the CD30 positive group, which will provide at least 80% power. Calculations are based on a 2-sided alpha level of 0.05 using the log-rank test.

The PFS rates in the control arm are based on (Czuczman, et al. 2017 and Wang M, et al. Leukemia 2013; approved online. Doi; 10.1038/leu.2013.95). Assuming a hazard ratio of 0.62 for both ITT and CD30+ populations, and a 5% annual dropout rate, approximately 400 subjects will be randomized in order to observe 280 PFS events within a reasonable timeframe.

The interim analysis of ORR will be conducted once enrollment has been completed and at least 250 subjects have completed 6 months of follow-up. It is anticipated that approximately 250 subjects will have completed 6 months of follow-up at the time of enrollment completion. Assuming an ORR of 57% in the experimental arm and an ORR of 28% in the control arm, this will provide at least 90% power to detect a difference in ORR between the 2 arms, based on Fisher's exact test at a 2-sided alpha of 0.005. An ORR of 57% for the experimental arm is based on the ongoing study being conducted by the Washington University School of Medicine (NCT02086604) as well as Wang et al. 2013, and the ORR of 28% for the control arm is based on Czuczman et al. (2017) and Wang et al. (2013).

The key secondary endpoint of OS will be tested in the ITT population and the CD30-positive population at 2 time points, provided there is a positive result for the primary endpoint for both the ITT population and the CD30-positive population. An interim analysis will be conducted at the time of the primary PFS analysis and a final analysis will be conducted after 300 OS events have been observed. Power for the ITT and CD30-positive populations are similar to those for the PFS analysis described above.

### Efficacy Analysis

### Primary Efficacy Analysis

Kaplan-Meier methods will be used to assess PFS. The stratified log-rank test will be used in the primary evaluation of PFS differences between the experimental arm and the control arm. The primary endpoint of PFS will be tested in the ITT population and CD30 positive populations, and an exhaustive fallback testing approach will be used to control overall type I error rate at a local 2-sided alpha of 0.05 (assigning α₁=0.03 to PFS in ITT, and α₂=0.02 to PFS in CD30 positive). All events entered in the database by the time of data cutoff that have been source data-verified will be included in the analysis of PFS, even if there are more than the prespecified number of events.

Kaplan-Meier Curves depicting PFS in the 2 arms will be generated, with median PFS reported by treatment group. The 2-sided 95% confidence intervals (CI) for the median will be calculated using the complementary log-log transformation method.

### Secondary Efficacy Analysis

OS will be analyzed using Kaplan-Meier methodology and Kaplan-Meier plots will be provided by treatment group using the ITT analysis set. OS will be tested when the PFS endpoint is positive for both the ITT population and the CD30-positive population. An interim and final analysis of OS are planned. The stratified log-rank test will be used in the primary evaluation of OS. The median OS and its 2-sided 95% CI using the complementary log-log transformation method will be calculated by treatment group. Duration of response will be analyzed similarly for the population of subjects achieving a CR or PR.

ORR will be summarized by treatment group using the ITT analysis set. The interim analysis of ORR will include all subjects who have completed at least 6 months of follow-up from the date of randomization or have discontinued from the study at the time of the analysis. ORR will be tested based on the Cochran Mantel-Haenszel test, stratified by stratification factors; the difference in ORR will be presented with the corresponding 95% confidence interval. ORR for each arm will also be presented with the exact 95% CI using the Clopper-Pearson method. Similar summaries will be presented for CR rate.

What is claimed is:

1. A method of treating non-Hodgkin lymphoma in a subject, the method comprising administering to the subject lenalidomide, or salt or solvate thereof, and an antibody-drug conjugate that binds to CD30, wherein the antibody-drug conjugate comprises an anti-CD30 antibody or an antigen-binding fragment thereof conjugated to a monomethyl auristatin or a functional analog thereof or a functional derivative thereof.

2. The method of claim 1, wherein the non-Hodgkin lymphoma is diffuse large B-cell lymphoma (DLBCL).

3. The method of claim 2, wherein the DLBCL is relapsed DLBCL.

4. The method of claim 2 or 3, wherein the DLBCL is refractory DLBCL.

5. The method of any one of claims 2-4, wherein the DLBCL is germinal-center B-cell like (GCB).

6. The method of any one of claims 2-4, wherein the DLBCL is non-GCB.

7. The method of any one of claims 1-6, wherein the subject has previously received allogenic stem cell transplant to treat the non-Hodgkin lymphoma.

8. The method of any one of claims 1-7, wherein the subject has previously received autologous stem cell transplant to treat the non-Hodgkin lymphoma.

9. The method of claim 7 or claim 8, wherein the subject relapsed following stem cell transplant.

10. The method of any one of claims 1-9, wherein the subject has previously received CAR-T therapy.

11. The method of claim 10, wherein the subject relapsed after CAR-T therapy.

12. The method of any one of claims 1-11, wherein the subject has not been previously treated with lenalidomide, or salt or solvate thereof.

13. The method of any one of claims 1-12, wherein the subject has not been previously treated with an antibody-drug conjugate that binds to CD30.

14. The method of any one of claims 1-13, wherein the non-Hodgkin lymphoma is an advanced stage non-Hodgkin lymphoma.

15. The method of claim 14, wherein the advanced stage non-Hodgkin lymphoma is a stage 3 or stage 4 non-Hodgkin lymphoma.

16. The method of claim 14 or claim 15, wherein the advanced stage non-Hodgkin lymphoma is metastatic non-Hodgkin lymphoma.

17. The method of any one of claims 1-16, wherein the non-Hodgkin lymphoma is recurrent non-Hodgkin lymphoma.

18. The method of any one of claims 1-17, wherein at least 1% of the non-Hodgkin lymphoma cells in the subject express CD30.

19. The method of any one of claims 1-18, wherein the anti-CD30 antibody of the antibody-drug conjugate comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6.

20. The method of any one of claims 1-19, wherein the anti-CD30 antibody of the antibody-drug conjugate comprises a heavy chain variable region comprising an amino acid sequence at least 85% identical to the amino acid sequence of SEQ ID NO: 7 and a light chain variable region comprising an amino acid sequence at least 85% identical to the amino acid sequence of SEQ ID NO: 8.

21. The method of any one of claims 1-20, wherein the anti-CD30 antibody of the antibody-drug conjugate comprises a heavy chain variable region comprising an amino acid sequence at least 90% identical to the amino acid sequence of SEQ ID NO: 7 and a light chain variable region comprising an amino acid sequence at least 90% identical to the amino acid sequence of SEQ ID NO: 8.

22. The method of any one of claims 1-21, wherein the anti-CD30 antibody of the antibody-drug conjugate comprises a heavy chain variable region comprising an amino acid sequence at least 95% identical to the amino acid sequence of SEQ ID NO: 7 and a light chain variable region comprising an amino acid sequence at least 95% identical to the amino acid sequence of SEQ ID NO: 8.

23. The method of any one of claims 1-18, wherein the anti-CD30 antibody of the antibody-drug conjugate comprises a heavy chain variable region comprising the amino acid sequence of SEQ ID NO: 7 and a light chain variable region comprising the amino acid sequence of SEQ ID NO:

8.

24. The method of any one of claims 1-18, wherein the anti-CD30 antibody is AC10.

25. The method of any one of claims 1-18, wherein the anti-CD30 antibody is cAC10.

26. The method of any one of claims 1-25, wherein the antibody-drug conjugate further comprises a linker between the anti-CD30 antibody or antigen-binding portion thereof and the monomethyl auristatin.

27. The method of claim 26, wherein the linker is a cleavable peptide linker.

28. The method of claim 27, wherein the cleavable peptide linker has a formula: -MC-vc-PAB-, wherein:
a) MC is: 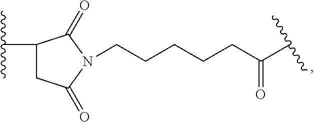 [SMILES: *C(=O)CCCCCN1C(=O)CC(*)C1=O]
b) vc is the dipeptide valine-citrulline, and
c) PAB is:

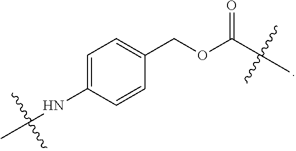 [SMILES: *Nc1ccc(COC(*)=O)cc1]

29. The method of any one of claims 1-28, wherein the monomethyl auristatin is monomethyl auristatin E (MMAE).

30. The method of any one of claims 1-28, wherein the monomethyl auristatin is monomethyl auristatin F (MMAF).

31. The method of any one of claims 1-18, wherein the antibody-drug conjugate is brentuximab vedotin or a biosimilar thereof.

32. The method of any one of claims 1-18, wherein the antibody-drug conjugate is brentuximab vedotin.

33. The method of any one of claims 1-32, wherein the lenalidomide, or salt or solvate thereof, is administered at a dose of 1 to 30 mg.

34. The method of claim 33, wherein the lenalidomide, or salt or solvate thereof, is administered at a dose of 20 mg.

35. The method of any one of claims 1-34, wherein the lenalidomide, or salt or solvate thereof, is administered orally.

36. The method of any one of claims 1-35, wherein the lenalidomide, or salt or solvate thereof, is administered about once per day.

37. The method of any one of claims 1-35, wherein the lenalidomide, or salt or solvate thereof, is administered once per day.

38. The method of any one of claims 1-37, wherein the antibody-drug conjugate is administered at a dose of 0.6 mg/kg to 2.3 mg/kg of the subject's bodyweight.

39. The method of claim 38, wherein the antibody-drug conjugate is administered at a dose of about 0.9 mg/kg of the subject's bodyweight.

40. The method of claim 38, wherein the antibody-drug conjugate is administered at a dose of 0.9 mg/kg of the subject's bodyweight.

41. The method of claim 38, wherein the antibody-drug conjugate is administered at a dose of about 1.2 mg/kg of the subject's bodyweight.

42. The method of claim 38, wherein the antibody-drug conjugate is administered at a dose of 1.2 mg/kg of the subject's bodyweight.

43. The method of claim 38, wherein the antibody-drug conjugate is administered to subjects having a bodyweight of greater than 100 kg as if the subject had a bodyweight of 100 kg.

44. The method of any one of claims 1-43, wherein the antibody-drug conjugate is administered to the subject once about every 3 weeks.

45. The method of any one of claims 1-43, wherein the antibody-drug conjugate is administered to the subject once every 3 weeks.

46. The method of any one of claims 1-45, wherein the antibody-drug conjugate is administered to the subject on about day 1 of about a 21-day treatment cycle.

47. The method of any one of claims 1-45, wherein the antibody-drug conjugate is administered to the subject on day 1 of a 21-day treatment cycle.

48. The method of any one of claims 1-47, wherein the antibody-drug conjugate is administered by intravenous infusion.

49. The method of any one of claims 1-48 further comprising the administration of an anti-CD20 antibody or antigen-binding fragment thereof to the subject.

50. The method of claim 49, wherein the anti-CD20 antibody or antigen-binding fragment thereof comprises a heavy chain variable region comprising the three CDRs of SEQ ID NO:17, a light chain variable region comprising the three CDRs of SEQ ID NO:18, wherein the CDRs of the anti-CD20 antibody are defined by the Kabat numbering scheme.

51. The method of claim 49 or 50, wherein the anti-CD20 antibody or antigen-binding fragment thereof comprises a heavy chain variable region comprising an amino acid sequence at least 85% identical to the amino acid sequence of SEQ ID NO:17 and a light chain variable region comprising an amino acid sequence at least 85% identical to the amino acid sequence of SEQ ID NO:18.

52. The method of claim 49, wherein the anti-CD20 antibody or antigen-binding fragment thereof comprises a heavy chain variable region comprising the amino acid sequence of SEQ ID NO:17 and a light chain variable region comprising the amino acid sequence of SEQ ID NO:18.

53. The method of claim 49, wherein the anti-CD20 antibody or antigen-binding fragment thereof is rituximab or a biosimilar thereof.

54. The method of claim 49, wherein the anti-CD20 antibody or antigen-binding fragment thereof is rituximab.

55. The method of any one of claims 49-54, wherein the anti-CD20 antibody or antigen-binding fragment thereof is administered at a dose of 100 mg/m² to 500 mg/m² of the subject's body surface area.

56. The method of claim 55, wherein the anti-CD20 antibody or antigen-binding fragment thereof is administered at a dose of about 375 mg/m² of the subject's body surface area.

57. The method of claim 55, wherein the anti-CD20 antibody or antigen-binding fragment thereof is administered at a dose of 375 mg/m² of the subject's body surface area.

58. The method of any one of claims 49-54, wherein the anti-CD20 antibody or antigen-binding fragment thereof is administered at a dose of 500 mg to 2000 mg.

59. The method of claim 58, wherein the anti-CD20 antibody or antigen-binding fragment thereof is administered at a dose of about 1400 mg.

60. The method of claim 58, wherein the anti-CD20 antibody or antigen-binding fragment thereof is administered at a dose of 1400 mg.

61. The method of any one of claims 49-60, wherein the anti-CD20 antibody or antigen-binding fragment thereof is administered to the subject once about every 3 weeks.

62. The method of any one of claims 49-60, wherein the anti-CD20 antibody or antigen-binding fragment thereof is administered to the subject once every 3 weeks.

63. The method of any one of claims 49-62, wherein the anti-CD20 antibody or antigen-binding fragment thereof is administered to the subject on about day 1 of about a 21-day treatment cycle.

64. The method of any one of claims 49-62, wherein the anti-CD20 antibody or antigen-binding fragment thereof is administered to the subject on day 1 of a 21-day treatment cycle.

65. The method of any one of claims 49-64, wherein the anti-CD20 antibody or antigen-binding fragment thereof is administered by intravenous infusion.

66. The method of any one of claims 49-64, wherein the anti-CD20 antibody or antigen-binding fragment thereof is administered by subcutaneous injection.

67. The method of any one of claims 63-66, wherein the anti-CD20 antibody or antigen-binding fragment thereof is administered at a dose of about 375 mg/m² of the subject's body surface area by intravenous infusion on about day 1 of the first 21-day treatment cycle and is administered at a dose of about 1400 mg by subcutaneous injection on about day 1 of each 21-day treatment cycle thereafter.

68. The method of any one of claims 63-66, wherein the anti-CD20 antibody or antigen-binding fragment thereof is administered at a dose of 375 mg/m² of the subject's body surface area by intravenous infusion on day 1 of the first 21-day treatment cycle and is administered at a dose of 1400 mg by subcutaneous injection on day 1 of each 21-day treatment cycle thereafter.

69. The method of any one of claims 1-68 further comprising the administration of granulocyte-colony stimulating factor (G-CSF) to the subject.

70. The method of claim 69, wherein the G-CSF is administered 1 to 3 days after the administration of the anti-CD30 antibody-drug conjugate.

71. The method of claim 69 or 70, wherein the G-CSF is selected from the group consisting of filgrastim, PEG-filgrastim, lenograstim, and tbo-filgrastim.

72. The method of any one of claims 1-71, wherein administering the lenalidomide, or salt or solvate thereof, and the antibody-drug conjugate that binds to CD30 to the subject results in a depletion of cancer cells by at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or about 100% compared to the amount of cancer cells before administering the lenalidomide, or salt or solvate thereof, and/or the antibody-drug conjugate that binds to CD30 to the subject.

73. The method of any one of claims 1-72, wherein one or more therapeutic effects in the subject is improved after administration of the lenalidomide, or salt or solvate thereof, and the antibody-drug conjugate that binds to CD30 relative to a baseline.

74. The method of claim 73, wherein the one or more therapeutic effects is selected from the group consisting of: objective response rate, duration of response, time to response, progression free survival and overall survival.

75. The method of any one of claims 1-74, wherein the objective response rate is at least about 20%, at least about 25%, at least about 30%, at least about 35%, at least about 40%, at least about 45%, at least about 50%, at least about 60%, at least about 70%, or at least about 80%.

76. The method of any one of claims 1-75, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the lenalidomide, or salt or solvate thereof, and/or the antibody-drug conjugate that binds to CD30.

77. The method of any one of claims 1-76, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the lenalidomide, or salt or solvate thereof, and/or the antibody-drug conjugate that binds to CD30.

78. The method of any one of claims 1-77, wherein the duration of response to the lenalidomide, or salt or solvate thereof, and/or the antibody-drug conjugate that binds to CD30 is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the lenalidomide, or salt or solvate thereof, and/or the antibody-drug conjugate that binds to CD30.

79. The method of any one of claims 1-78, wherein the subject is a human.

80. A pharmaceutical composition for the treatment of non-Hodgkin lymphoma in a subject, the composition comprising lenalidomide, or salt or solvate thereof, and an antibody-drug conjugate that binds to CD30, wherein the antibody-drug conjugate comprises an anti-CD30 antibody or an antigen-binding fragment thereof conjugated to a monomethyl auristatin or a functional analog thereof or a functional derivative thereof, wherein the composition is for use in a method of any one of claims 1-79.

81. The pharmaceutical composition of claim 80 further comprising an anti-CD20 antibody or antigen-binding fragment thereof.

82. A kit comprising lenalidomide, or salt or solvate thereof, and an antibody-drug conjugate that binds to CD30, wherein the antibody-drug conjugate comprises an anti-CD30 antibody or an antigen-binding fragment thereof conjugated to a monomethyl auristatin or a functional analog thereof or a functional derivative thereof, and instructions for using the kit in the method of any one of claims 1-79.

83. The kit of claim 82 further comprising an anti-CD20 antibody or antigen-binding fragment thereof.


SEQUENCE LISTING (20 sequences)

SEQ ID NO: 1 - protein, 5 aa, Artificial Sequence
  Synthetic Construct
DYYIT

SEQ ID NO: 2 - protein, 17 aa, Artificial Sequence
  Synthetic Construct
WIYPGSGNTKYNEKFKG

SEQ ID NO: 3 - protein, 8 aa, Artificial Sequence
  Synthetic Construct
YGNYWFAY

SEQ ID NO: 4 - protein, 15 aa, Artificial Sequence
  Synthetic Construct
KASQSVDFDGDSYMN

SEQ ID NO: 5 - protein, 7 aa, Artificial Sequence
  Synthetic Construct
AASNLES

SEQ ID NO: 6 - protein, 9 aa, Artificial Sequence
  Synthetic Construct
QQSNEDPWT

SEQ ID NO: 7 - protein, 117 aa, Artificial Sequence
  Synthetic Construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 8 - protein, 111 aa, Artificial Sequence
  Synthetic Construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 9 - protein, 30 aa, Artificial Sequence
  Synthetic Construct
QIQLQQSGPEVVKPGASVKISCKASGYTFT

SEQ ID NO: 10 - protein, 14 aa, Artificial Sequence
  Synthetic Construct
WVKQKPGQGLEWIG

SEQ ID NO: 11 - protein, 32 aa, Artificial Sequence
  Synthetic Construct
KATLTVDTSSSTAFMQLSSLTSEDTAVYFCAN

SEQ ID NO: 12 - protein, 11 aa, Artificial Sequence
  Synthetic Construct
WGQGTQVTVSA

SEQ ID NO: 13 - protein, 23 aa, Artificial Sequence
  Synthetic Construct
DIVLTQSPASLAVSLGQRATISC

SEQ ID NO: 14 - protein, 15 aa, Artificial Sequence
  Synthetic Construct
WYQQKPGQPPKVLIY

SEQ ID NO: 15 - protein, 32 aa, Artificial Sequence
  Synthetic Construct
GIPARFSGSGSGTDFTLNIHPVEEEDAATYYC

SEQ ID NO: 16 - protein, 10 aa, Artificial Sequence
  Synthetic Construct
FGGGTKLEIK

SEQ ID NO: 17 - protein, 120 aa, Artificial Sequence
  Synthetic Construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVS

SEQ ID NO: 18 - protein, 106 aa, Artificial Sequence [sequence omitted]

SEQ ID NO: 19 - protein, 451 aa, Artificial Sequence
  Synthetic Construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 213 aa, Artificial Sequence
  Synthetic Construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC